(19) United States
(12) Patent Application Publication
(10) Pub. No.: US 2012/0035161 A1
(43) Pub. Date: Feb. 9, 2012

(54) Novel Compositions and Methods of Treating Diseases Using the Same

(75) Inventors: Stephen Roth, Gladwyne, PA (US); Bruce Tomczuk, Collegeville, PA (US)

(73) Assignee: CORRIDOR PHARMACEUTICALS, INC.

(21) Appl. No.: 13/185,161

(22) Filed: Jul. 18, 2011

Related U.S. Application Data
(63) Continuation-in-part of application No. 11/897,598, filed on Aug. 31, 2007, now Pat. No. 7,981,885.
(60) Provisional application No. 60/841,771, filed on Sep. 1, 2006.

(51) Int. Cl. A61K 31/5415; C12N 5/02; A61P 29/00; C07D 417/06; A61P 11/06

(52) U.S. Cl. 514/225.5; 544/44; 514/225.8; 544/45; 544/42; 544/46; 435/375

(57) ABSTRACT

The invention includes compositions and methods for inhibiting proliferation and inducing apoptosis in activated lymphocytes, treating diseases associated with activated lymphocytes, or treating PAH.

### CROSS-REFERENCE TO RELATED APPLICATION

The present application is a continuation-in-part of and claims priority to U.S. patent application Ser. No. 11/897,598, filed Aug. 31, 2007, which issued as U.S. Pat. No. 7,981,885 on Jul. 19, 2011, which is entitled to priority under 35 U.S.C. §119(e) to U.S. Provisional Application No. 60/841,771, filed Sep. 1, 2006, which applications are incorporated by reference herein in their entireties.

### BACKGROUND OF THE INVENTION

Serotonin (also referred to as 5-hydroxytryptamine or 5-HT) is a neurotransmitter that has been strongly implicated in the pathophysiology and treatment of a wide variety of neuropsychiatric disorders. Serotonin exerts its effects through a diverse family of serotonin receptor molecules (referred to herein as “5-HT receptors” or “5-HTRs”). Classically, members of the serotonin receptor family have been grouped into seven (7) subtypes pharmacologically, i.e., according to their specificity of various serotonin antagonists. Thus, while all the 5-HT receptors specifically bind with serotonin, they are pharmacologically distinct and are encoded by separate genes. To date, fourteen (14) mammalian serotonin receptors have been identified and sequenced. More particularly, these fourteen separate 5-HT receptors have been grouped into seven (7) pharmacological subtypes, designated 5-HT1, 5-HT2, 5-HT3, 5-HT4, 5-HT5, 5-HT6, and 5-HT7. Several of the subtypes are further subdivided such that the receptors are grouped pharmacologically as follows: 5-HT1A, 5-HT1B, 5-HT1D, 5-HT1E, 5-HT1F, 5-HT2A, 5-HT2B, 5-HT2C, 5-HT3A, 5-HT3B, 5-HT4, 5-HT5A, 5-HT6, 5-HT7. However, when the nucleic and amino acid sequences of the receptors are compared, the percent identity among the subtypes is not correlated to the pharmacological groupings.

Of the fourteen different mammalian serotonin receptors that have been cloned, all but one are members of the G-protein coupled receptor superfamily. Serotonin receptors 5-HT1A, 5-HT1B, and 5-HT1D inhibit adenylate cyclase, and 5-HT2 receptors activate phospholipase C pathways, stimulating breakdown of polyphosphoinositides. The 5-HT2 receptor belongs to the family of rhodopsin-like signal transducers that are distinguished by a seven-transmembrane configuration and functional linkage to G-proteins. The 5-HT3 receptor family includes ligand-gated ion channel receptors that have four putative TMDs.

Serotonin regulates a wide variety of sensory, motor and behavioral functions in the mammalian CNS, including behaviors such as learning and memory, sleep, thermoregulation, motor activity, pain, sexual and aggressive behaviors, appetite, neuroendocrine regulation, and biological rhythms. Serotonin has also been linked to pathophysiological conditions such as anxiety, depression, obsessive-compulsive disorders, schizophrenia, suicide, autism, migraine, emesis, alcoholism and neurodegenerative disorders. This biogenic amine neurotransmitter is synthesized by neurons of the brain stem that project throughout the CNS, with highest density in basal ganglia and limbic structures (Steinbusch, 1984, In: Handbook of Chemical Neuroanatomy 3:68-125, Bjorklund et al., Eds., Elsevier Science Publishers, B.V.).

Studies have suggested that serotonin may play a role in the immune system since data demonstrate that serotonin receptors are present on various cells of the immune system. There have been reports in the literature about the immunomodulatory effects of adding serotonin exogenously to mitogenically stimulated lymphocyte cultures. Under some circumstances, serotonin has been shown to stimulate the activated T cells (Foon et al., 1976, J. Immunol. 117:1545-1552; Kut et al., 1992, Immunopharmacol. Immunotoxicol. 14:783-796; Young et al., 1993, Immunology 80:395-400), whereas other laboratories report that high concentrations of added serotonin inhibit the proliferation (Slauson et al., 1984, Cell. Immunol. 84:240-252; Khan et al., 1986, Int. Arch. Allergy Appl. Immunol. 81:378-380; Mossner & Lesch, 1998, Brain, Behavior, and Immunity 12:249-271).

Of the fourteen known pharmacologically distinct serotonin receptors, lymphocytes express type 2A, type 2B, type 2C, type 6 and type 7 on resting cells (Ameisen et al., 1989, J. Immunol. 142:3171-3179; Stefulj et al., 2000, Brain, Behavior and Immunity 14:219-224) and that the type 1A and type 3 receptors are up-regulated upon activation (Aune et al., 1993, J. Immunol. 151:1175-1183; Meyniel et al., 1997, Immunol. Lett. 55:151-160; Stefulj et al., 2000, Brain, Behavior, and Immunity 14:219-224).

The involvement of the 5-HT1A receptors in human and murine T cells has also been demonstrated (Anne et al., 1990, J. Immunol. 145:1826-1831; Aune et al., 1993, J. Immunol. 151:1175-1183; Aune et al., 1994, J. Immunol. 153:1826-1831). These studies established that IL-2-stimulated human T cell proliferation could be inhibited by a blockade of tryptophan hydroxylase, i.e., the first enzyme involved in the conversion of tryptophan to serotonin, and that the inhibition could be reversed by the addition of 5-hydroxy tryptophan. Furthermore, human T cell proliferation was blocked in vitro with a 5-HT1A-specific receptor antagonist. In a murine model, a type 1A receptor antagonist, but not a type 2 receptor antagonist, was able to inhibit the in vivo contact sensitivity response, but not antibody responses, to oxazalone. PCT Publication No. WO 03/106660 discloses the use of fluphenazine, an antagonist of 5-HT(1B/1D) and 5-HT(2C) receptors, for inhibiting proliferation and inducing cell death in lymphocytes.

Pulmonary hypertension (PH) is a disease associated with an increase in blood pressure in the pulmonary artery, pulmonary vein, or pulmonary capillaries (together known as the lung vasculature), leading to shortness of breath, dizziness, fainting and other symptoms, all of which are exacerbated by exertion. Pulmonary hypertension may be a severe disease with a markedly decreased exercise tolerance and heart failure. It may be one of five different types: arterial, venous, hypoxic, thromboembolic or miscellaneous.

In pulmonary arterial hypertension (PAH), the pressure in a patient's pulmonary arteries becomes dangerously high, straining the heart. PAH worsens over time and is life-threatening. There are several types of PAH: (a) idiopathic, of unknown cause; (b) familial, often linked to a genetic defect; (c) associated, the most common type, and linked with medical conditions including: collagen vascular disease (or connective tissue disease, including autoimmune diseases such as scleroderma or lupus), congenital heart and lung disease, portal hypertension (usually resulting from liver disease), HIV infection, drugs (including appetite suppressants, particularly fenfluramine and dexfenfluramine, cocaine or amphetamines, and other drugs), and other conditions such as thyroid disorders, glycogen storage disease (a genetic defect in forming or releasing sugars necessary for the body to function), Gaucher disease, hereditary hemorrhagic telangiectasia (abnormally formed blood vessels resulting in excessive bleeding), hemoglobinopathies (an abnormally formed oxygen carrying protein in the red blood cells, caused by a genetic defect), myeloproliferative disorders (an overproduction of red or white blood cells) and splenectomy (removal of the spleen); (d) associated with significant venous or capillary involvement (which occurs at the same time as abnormal narrowing in the pulmonary veins and/or capillaries and may include arteries), including pulmonary veno-occlusive disease (resulting in blockage of the veins in the lungs) and pulmonary capillary hemangiomatosis (wherein small blood vessels in the lungs grow too much and become tangled, resulting in poor blood flow); and (e) persistent pulmonary hypertension of the newborn (wherein a newborn's heart and blood vessels do not adapt to breathing outside the womb).

PAH may be caused by contraction of muscles within the walls of the arteries; thickening of walls of the arteries; or formation of tiny blood clots within the smaller arteries. Any of these changes makes it difficult for blood to pass through the lungs, forcing the heart to work too hard. Over time, the heart muscle weakens and can no longer pump blood efficiently. At that pont, patients with PAH experience symptoms such as shortness of breath, fatigue, chest pain, dizziness, and fainting. If PAH is not treated, the heart fails eventually, leading to severe disability and even death. There is no cure for PAH, and approximately 50% of people diagnosed with PAH die within five years. For people whose PAH is not treated, average survival is only about three years. Even with treatment, the pressure in the lungs caused by PAH continues to worsen and make performance of everyday tasks more difficult.

There is a long-felt need in the art to develop novel compounds and therapies for treating diseases related to activated lymphocytes and lymphocyte proliferation, especially diseases related to activated T cells and B cells. In addition, there is a long-felt need to develop novel compounds without the side effects related to other serotonin receptor antagonists. Furthermore, there is a long-felt need to develop novel compounds for preventing or treating PAH in patients in need thereof. The present invention meets these needs.

### BRIEF SUMMARY OF THE INVENTION

The present invention includes a compound of formula I or a pharmaceutically acceptable salt, prodrug or solvate thereof:

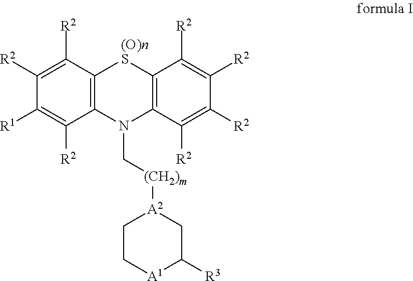

wherein:

each occurrence of R¹ is independently selected from the group consisting of hydrogen, halogen, (C₁-C₆)alkyl; (C₁-C₆)alkenyl; (C₁-C₆)alkoxy; OH; NO₂; C≡N; C(═O)OR⁷; C(═O)NR⁷₂; NR⁷₂; NR⁷C(═O)(C₁-C₆)alkyl; NR⁷C(═O)O(C₁-C₆)alkyl; NR⁷C(═O)NR⁷₂; NR⁷SO₂(C₁-C₆)alkyl; SO₂NR⁷₂; OC(═O)(C₁-C₆)alkyl; O(C₂-C₆)alkylene-NR⁷₂; (C₂-C₆)alkylene-OR⁷; and (C₁-C₃)perfluoroalkyl;

each occurrence of R² is independently selected from the group consisting of hydrogen, halogen, (C₁-C₆)alkyl; (C₁-C₆)alkenyl; (C₁-C₆)alkoxy; OH; NO₂; C≡N; C(═O)OR⁷; C(═O)NR⁷₂; NR⁷₂; NR⁷C(═O)(C₁-C₆)alkyl; NR⁷C(═P)O(C₁-C₆)alkyl; NR⁷C(═O)NR⁷₂; NR⁷SO₂(C₁-C₆)alkyl; SO₂NR⁷₂; OC(═O)(C₁-C₆)alkyl; O(C₂-C₆)alkylene-NR⁷₂; (C₂-C₆)alkylene-OR⁷; and (C₁-C₃)perfluoroalkyl;

R³ is hydrogen, C(═O)OR⁷, or C(═O)NR⁷₂;

A¹ is CH₂ or NR⁴;

A² is CH or N;

provided that if A¹ is CH₂, then A² is N, and if A² is CH, then A¹ is NR⁴;

R⁴ is H, (C₁-C₆)alkyl; (CH₂)ₚOR⁷; (CH₂)ₚNR⁷₂; (CH₂)ₚNR⁷C(O)R⁵; (CH₂)ₚO(CH₂)ₚOR⁷; (CH₂)ₚO(CH₂)ₚNR⁷₂; (CH₂)ₚNR⁴(CH₂)ₚNR⁷₂; (CH₂)ₚO(CH₂)ₚNHC(O)R⁵; (CH₂)ₚNR⁷(CH₂)ₚNHC(O)R⁵; (CH₂)_{q}C(═O)OR⁷; (CH₂)_{q}C(═O)NR⁷₂; (CH₂)ₚO(CH₂)_{q}C(═O)OR⁷; (CH₂)ₚO(CH₂)_{q}C(═O)NR⁷₂; (CH₂)ₚNR⁷(CH₂)_{q}C(═O)OR⁷; or (CH₂)ₚNR⁷(CH₂)_{q}C(═O)NR⁷₂;

R⁵ is H; (C₁-C₆)alkyl; CR⁸R⁹R¹⁰; NR⁷C(═O)(C₁-C₆)alkyl; NR⁷C(═O)O(C₁-C₆)alkyl; NR⁷C(═O)NR⁷₂; CH(R⁶)NR⁷₂; CH(R⁶)NR⁷C(═O)(C₁-C₆)alkyl; or CH(R⁶)NR⁷C(═O)O(C₁-C₆)alkyl;

R⁶ is H, (C₁-C₆)alkyl; (C₂-C₆)alkylene-OR⁷; (CH₂)_{q}C(═O)OR⁷; or (CH₂)_{q}C(═O)NR⁷₂;

each occurrence of R⁷ and R¹⁰ is independently selected from the group consisting of hydrogen, (C₁-C₆)cycloalkyl and (C₁-C₆)alkyl;

each occurrence of R⁸ and R⁹ is independently selected from the group consisting of (C₁-C₆)cycloalkyl and (C₁-C₆)alkyl;

m is independently at each occurrence 1, 2, or 3;

n is 0, 1, or 2;

p is independently at each occurrence 2 or 3; and

q is independently at each occurrence 1 or 2.

In one embodiment, R¹ is hydrogen, halogen, (C₁-C₆)alkyl, methyl, C≡N, C(═O)NR⁷₂, C(═O)NH₂, SO₂NR⁷₂, SO₂NMe₂, (C₁-C₃)perfluoroalkyl, or CF₃. In another embodiment, each occurrence of R² is hydrogen. In yet another embodiment, R³ is hydrogen. In yet another embodiment, A¹ is NR⁴. In yet another embodiment, A² is N.

In one embodiment, R⁴ is H, (CH₂)ₚNR⁷₂, CH₂CH₂NH₂, CH₂CH₂CH₂NH₂, (CH₂)ₚNR⁷C(O)R⁵, CH₂CH₂NHC(O)R⁵, CH₂CH₂NHC(O)Me, CH₂CH₂NHC(O)CH₂NH₂, or CH₂CH₂NHC(O)CH₂NMe. In another embodiment, R⁴ is (CH₂)ₚNR⁷C(O)R⁵. In yet another embodiment, R⁴ is (CH₂)ₚNHC(O)R⁵.

In one embodiment, R⁵ is (C₁-C₆)alkyl, CH(R⁶)NR⁷₂, or CH(R⁶)NH₂ or NHMe. In another embodiment, R⁵ is H or CR⁸R⁹R¹⁰.

In one embodiment, R⁶ is H. In yet another embodiment, m is 2, n is 0, p is 2, and q is 1.

The present invention also includes a compound of formula II or a pharmaceutically acceptable salt, prodrug or solvate thereof:

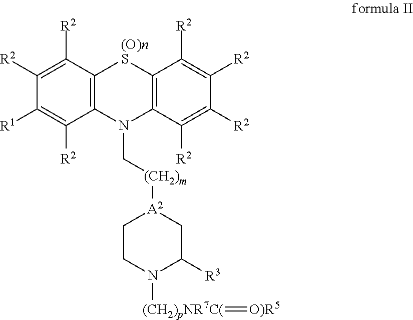

wherein:

each occurrence of R¹ and R² is independently selected from the group consisting of hydrogen, halogen, (C₁-C₆)alkyl; (C₁-C₆)alkenyl; (C₁-C₆)alkoxy; OH; NO₂; C≡N; C(═O)OR⁷; C(═O)NR⁷₂; NR⁷₂; NR⁷C(═O)(C₁-C₆)alkyl; NR⁷C(═O)O(C₁-C₆)alkyl; NR⁷C(═O)NR⁷₂; NR⁷SO₂(C₁-C₆)alkyl; SO₂NR⁷₂; OC(═O)(C₁-C₆)alkyl; O(C₂-C₆)alkylene-NR⁷₂; (C₂-C₆)alkylene-OR⁷; and (C₁-C₃)perfluoroalkyl;

R³ is hydrogen, C(═O)OR⁷, or C(═O)NR⁷₂;

A² is CH or N;

R⁵ is H or CR⁸R⁹R¹⁰;

each occurrence of R⁷ and R¹⁰ is independently selected from the group consisting of hydrogen, (C₁-C₆)cycloalkyl and (C₁-C₆)alkyl;

each occurrence of R⁸ and R⁹ is independently selected from the group consisting of (C₁-C₆)cycloalkyl and (C₁-C₆)alkyl; or R⁸ and R⁹ are bound to the same carbon atom and linked as to form a divalent group selected from the group consisting of ethane-1,2-diyl, propane-1,3-diyl, butane-1,4-diyl, pentane-1,5-diyl, hexane-1,6-diyl and heptane-17-diyl; wherein said bivalent group is optionally substituted with at least one (C₁-C₆)alkyl group;

m is independently at each occurrence 1, 2, or 3;

n is 0, 1, or 2;

p is independently at each occurrence 2 or 3; and

q is independently at each occurrence 1 or 2.

In one embodiment, the compound of formula II is N-(2-(4-(3-(2-(trifluoromethyl)-10H-phenothiazin-10-yl)propyl)piperazin-1-yl)ethyl)pivalamide (Compound 35b) or a salt thereof.

The present invention further includes a compound of formula III or a pharmaceutically acceptable salt, prodrug or solvate thereof:

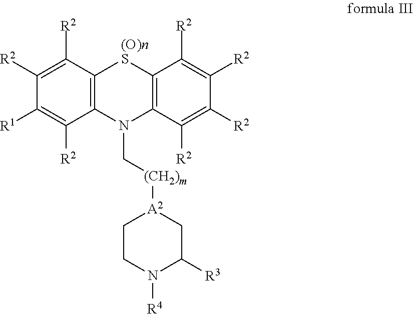

wherein:

each occurrence of R¹ and R² is independently selected from the group consisting of hydrogen, halogen, (C₁-C₆)alkyl; (C₁-C₆)alkenyl; (C₁-C₆)alkoxy; OH; NO₂; C≡N; C(═O)OR⁵; C(═O)NR⁵₂; NR⁵₂; NR⁵C(═O)(C₁-C₆)alkyl; NR⁵C(═O)O(C₁-C₆)alkyl; NR⁵C(═O)NR⁵₂; NR⁵SO₂(C₁-C₆)alkyl; SO₂NR⁵₂; OC(═O)(C₁-C₆)alkyl; O(C₂-C₆)alkylene-NR⁵₂; (C₂-C₆)alkylene-OR⁵; and (C₁-C₃)perfluoroalkyl;

R³ is hydrogen, C(═O)OR⁵, or C(═O)N(R⁵)₂;

A² is CH or N;

R⁴ is —(CR⁵₂)ₚN(R⁵)C(═O)—CR⁶R⁷R⁸; —(CR⁵₂)ₚO(CR⁵₂)ₚN(R⁵)C(═O)—CR⁶R⁷R⁸; —(CR⁵₂)ₚN(R⁵)(CR⁵₂)ₚN(R⁵)C(═O)—CR⁶R⁷R⁸; or —(CR⁵₂)ₚN(R⁵)C(═O)(CR⁵₂)ₚN(R⁵)C(═O)—CR⁶R⁷R⁸;

each occurrence of R⁵ and R⁶ is independently selected from the group consisting of hydrogen, (C₁-C₆)alkyl and (C₁-C₆)cycloalkyl;

R⁷ is (C₁-C₆)alkyl or (C₁-C₆)cycloalkyl; or R⁶ and R⁷ are bound to the same carbon atom and linked as to form a divalent group selected from the group consisting of ethane-1,2-diyl, propane-1,3-diyl, butane-1,4-diyl, pentane-1,5-diyl, hexane-1,6-diyl and heptane-17-diyl; wherein said bivalent group is optionally substituted with at least one (C₁-C₆)alkyl group;

R⁸ is (C₁-C₆)alkyl, —N(R⁵)C(═O)R⁵, or —N(R⁵)S(═O)₂R⁷;

m is independently at each occurrence 1, 2, or 3;

n is 0, 1, or 2; and,

p is independently at each occurrence 1, 2 or 3.

In one embodiment, R¹ is hydrogen, halogen, (C₁-C₆)alkyl, methyl, C≡N, C(═O)NR⁷₂, C(═O)NH₂, SO₂NR⁷₂, SO₂NMe₂, (C₁-C₃)perfluoroalkyl, or CF₃. In another embodiment, each occurrence of R² is hydrogen. In yet another embodiment, R³ is hydrogen.

In one embodiment, A² is N. In another embodiment, R⁴ is —(CR⁵₂)ₚN(R⁵)C(═O)—CR⁶R⁷R⁸. In yet another embodiment, m is 2 or 3. In yet another embodiment, n is 0. In yet another embodiment, p is 2. In yet another embodiment, R⁸ is (C₁-C₆)alkyl or —N(R⁵)(C═O)R⁵.

In one embodiment, the compound of formula III is selected from the group consisting of 2-amino-2-methyl-N-(2-(4-(3-(2-(trifluoromethyl)-10H-phenothiazin-10-yl)propyl)piperazin-1-yl)ethyl)propanamide (Compound 36a), 2-formamido-N-(2-(4-(3-(2-(trifluoromethyl)-10H-phenothiazin-10-yl)propyl)piperazin-1-yl)ethyl)acetamide (Compound 37b), a salt thereof, and mixtures thereof.

The present invention also includes a compound selected from the group consisting of ICI-681, ICI-682, ICI-683, ICI-684, ICI-685, ICI-686, ICI-687, ICI-696, ICI-697, ICI-712, ICI-713, and ICI-714, ICI-715, ICI-726, ICI-727, ICI-728, ICI-734, ICI-735, ICI-737, ICI-738, ICI-746, ICI-747, ICI-748, ICI-749, ICI-758, ICI-759, ICI-760, ICI-761, ICI-763, ICI-783, ICI-784, ICI-801, ICI-802, ICI-822, ICI-823, ICI-824, ICI-846, ICI-847, ICI-848, ICI-849, ICI-850, ICI-890, ICI-891, ICI-892, ICI-893, ICI-894, ICI-895, 2-amino-2-methyl-N-(2-(4-(3-(2-(trifluoromethyl)-10H-phenothiazin-10-yl)propyl)piperazin-1-yl)ethyl)propanamide (Compound 36a); N-(2-(4-(3-(2-(trifluoromethyl)-10H-phenothiazin-10-yl)propyl)piperazin-1-yl)ethyl)pivalamide (Compound 35b), 2-formamido-N-(2-(4-(3-(2-(trifluoromethyl)-10H-phenothiazin-10-yl)propyl)piperazin-1-yl)ethyl)acetamide (Compound 37b), and combinations thereof.

The present invention further includes a method of inducing apoptosis in an immune cell, wherein the method comprises contacting the immune cell with a composition comprising a compound of formula I, II or III.

In one embodiment, the immune cell is a lymphocyte. In another embodiment, the lymphocyte is selected from the group consisting of a T cell and a B cell. In yet another embodiment, the B cell is a plasma cell. In yet another embodiment, the plasma cell is a multiple myeloma cell.

The present invention also includes a method of inhibiting proliferation of a lymphocyte, wherein the method comprises contacting the lymphocyte with a composition comprising a compound of formula I, II or III.

The present invention further includes a method of treating a disease characterized by abnormal lymphocyte proliferation in a mammal, wherein the method comprises administering to the mammal a therapeutically effective amount of a pharmaceutically acceptable composition comprising a compound of formula I, II or III.

The invention also includes a method of treating a disease selected from the group consisting of asthma and rheumatoid arthritis in a mammal, wherein the method comprises administering to the mammal a therapeutically effective amount of a pharmaceutically acceptable composition comprising a compound of formula I, II or III.

The invention further includes a method of preventing or treating PAH in a mammal in need thereof, comprising treating the mammal with a therapeutically effective amount of a pharmaceutically acceptable composition comprising a compound of formula I, II or III.

In one embodiment, the mammal is a human.

### BRIEF DESCRIPTION OF THE DRAWINGS

For the purpose of illustrating the invention, there are depicted in the drawings certain embodiments of the invention. However, the invention is not limited to the precise arrangements and instrumentalities of the embodiments depicted in the drawings.

FIG. 1 is a graph depicting the results of an MTT assay demonstrating the inhibition of proliferation of HeLa cells using the indicated 5-HT receptor antagonists and the selective 5-HT1B receptor antagonist SB 216641.

FIG. 2 is a graph depicting the results of an MTT assay demonstrating the inhibition of proliferation of CCRF-CEM cells using the indicated 5-HT receptor antagonists and the selective 5-HT1B receptor antagonist SB 216641.

FIG. 3 is a graph depicting the results of an MTT assay demonstrating the inhibition of proliferation of RPMI-8226 cells using the indicated 5-HT receptor antagonists and the selective 5-HT1B receptor antagonist SB 216641.

FIG. 4 is a graph depicting the results of an MTT assay demonstrating the inhibition of proliferation of HeLa cells using the indicated 5-HT receptor antagonists and the selective 5-HT1B receptor antagonist SB 216641.

FIG. 5 is a graph depicting the results of an MTT assay demonstrating the inhibition of proliferation of CCRF-CEM cells using the indicated 5-HT receptor antagonists and the selective 5-HT1B receptor antagonist SB 216641.

FIG. 6 is a graph depicting the results of an MTT assay demonstrating the inhibition of proliferation of RPMI-8226 cells using the indicated 5-HT receptor antagonists and the selective 5-HT1B receptor antagonist SB 216641.

FIG. 7, comprising FIGS. 7A through 7F, is a series of images depicting the chemical structures of the following 5-HT receptor antagonists: ICI-681 (FIG. 7A), ICI-682 (FIG. 7B), ICI-683 (FIG. 7C), ICI-684 (FIG. 7D), ICI-685 (FIG. 7E), and ICI-686 (FIG. 7F).

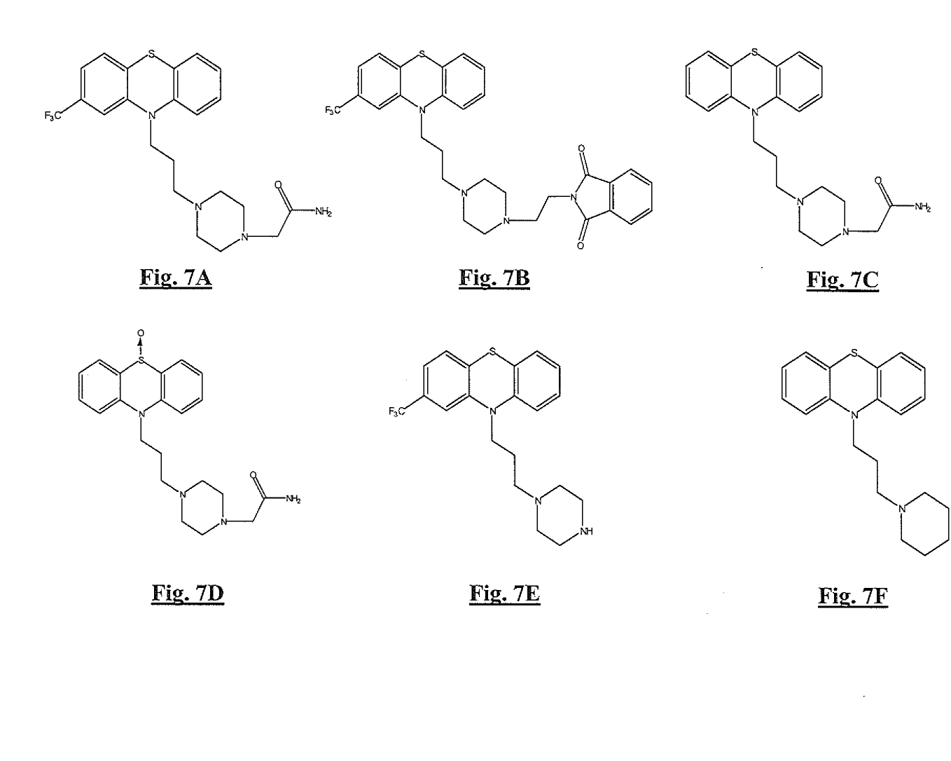

FIG. 8, comprising FIGS. 8A through 8F, is a series of images depicting the chemical structures of the following 5-HT receptor antagonists: ICI-687 (FIG. 8A), ICI-696 (FIG. 8B), ICI-697 (FIG. 8C), ICI-712 (FIG. 8D), ICI-713 (FIG. 8E), and ICI-714 (FIG. 8F).

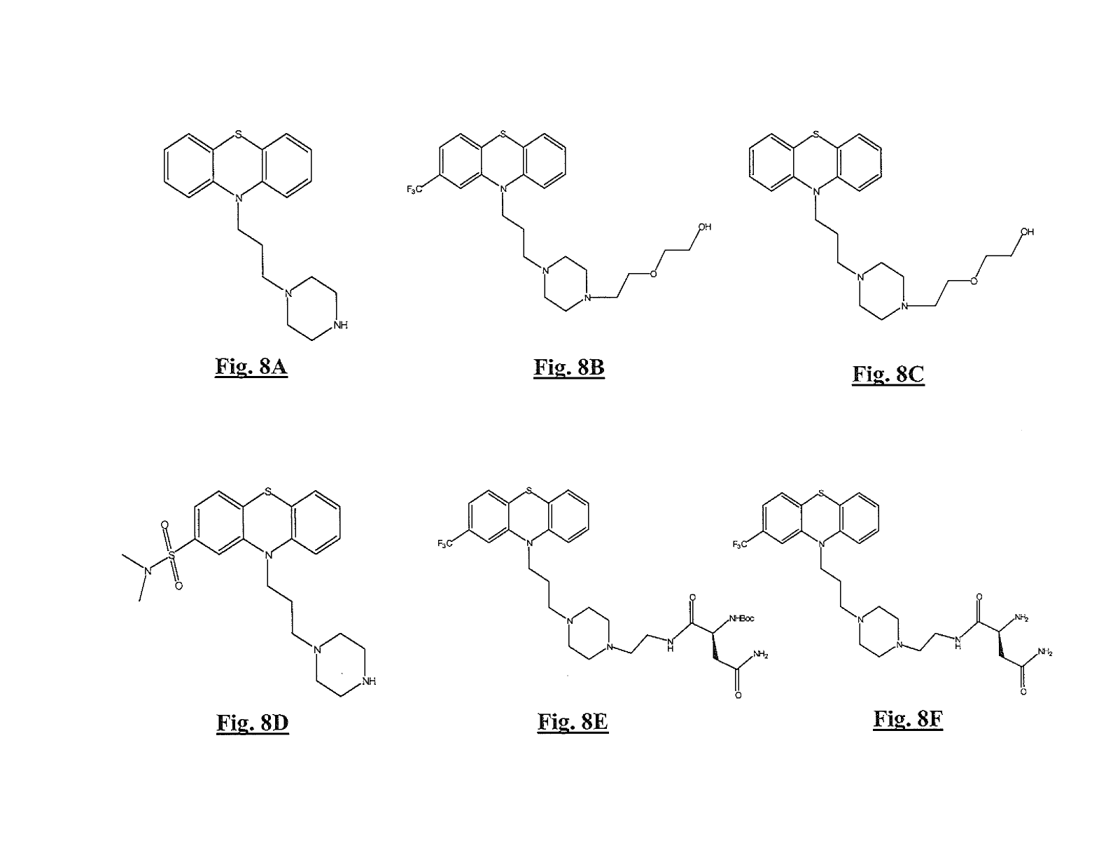

FIG. 9, comprising FIGS. 9A through 9F, is a series of images depicting the chemical structures of the following 5-HT receptor antagonists: ICI-715 (FIG. 9A), ICI-726 (FIG. 9B), ICI-727 (FIG. 9C), ICI-728 (FIG. 9D), ICI-734 (FIG. 9E), and ICI-735 (FIG. 9F).

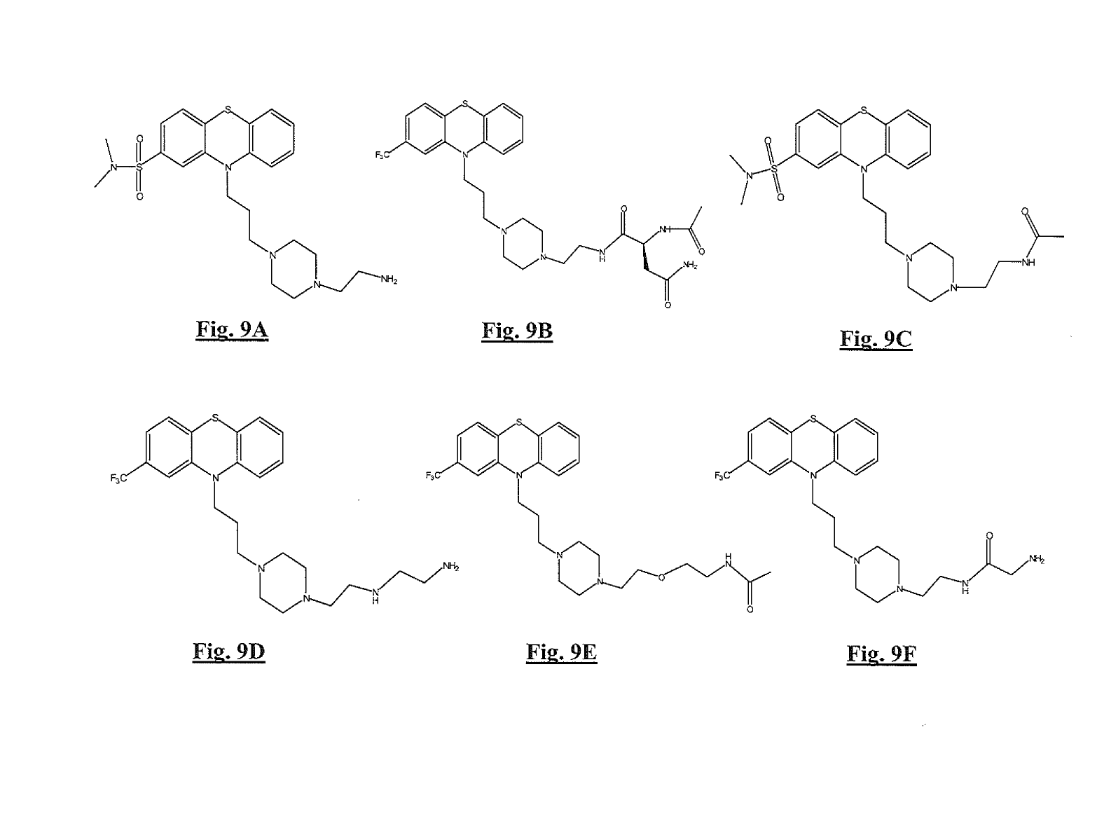

FIG. 10, comprising FIGS. 10A through 10F, is a series of images depicting the chemical structures of the following 5-HT receptor antagonists: ICI-737 (FIG. 10A), ICI-738 (FIG. 10B), ICI-746 (FIG. 10C), ICI-747 (FIG. 10D), ICI-748 (FIG. 10E), and ICI-749 (FIG. 10F),

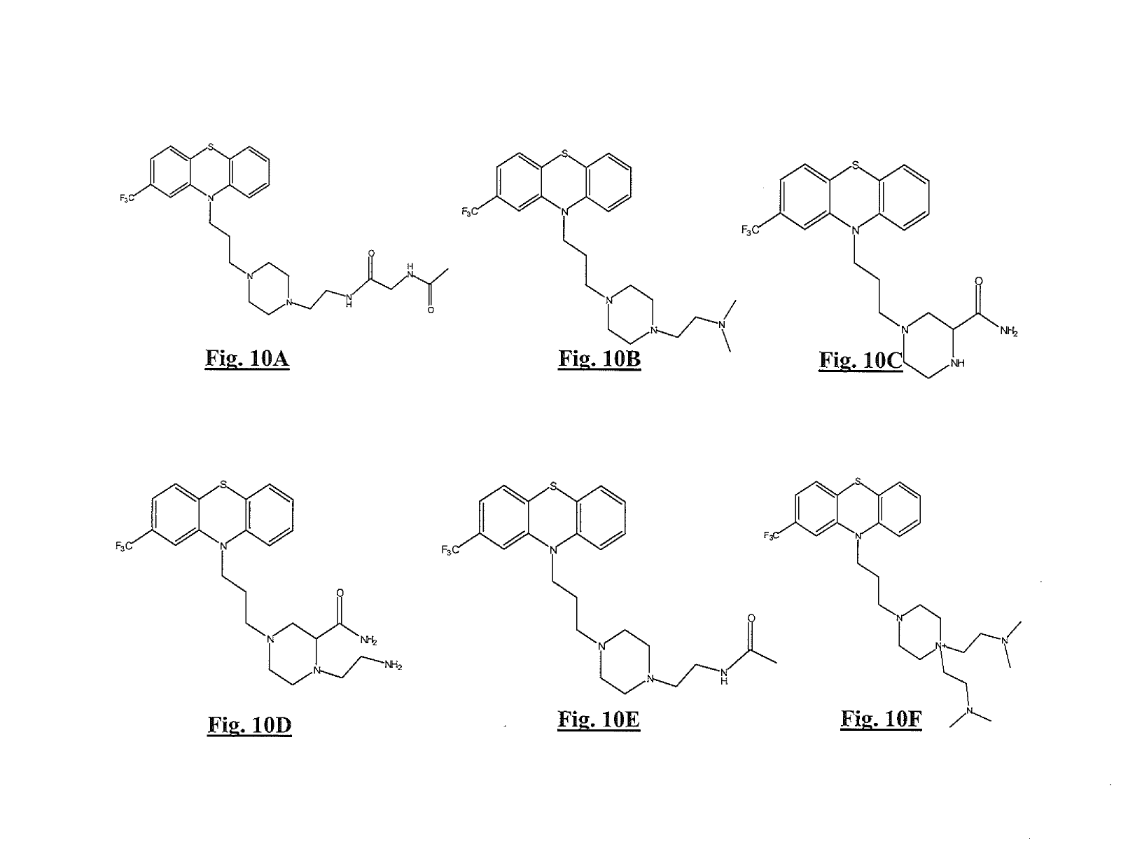

FIG. 11, comprising FIGS. 11A through 11F, is a series of images depicting the chemical structures of the following 5-HT receptor antagonists: ICI-758 (FIG. 11A), ICI-759 (FIG. 11B), ICI-760 (FIG. 11C), ICI-761 (FIG. 11D), ICI-763 (FIG. 11E), and ICI-783 (FIG. 11F).

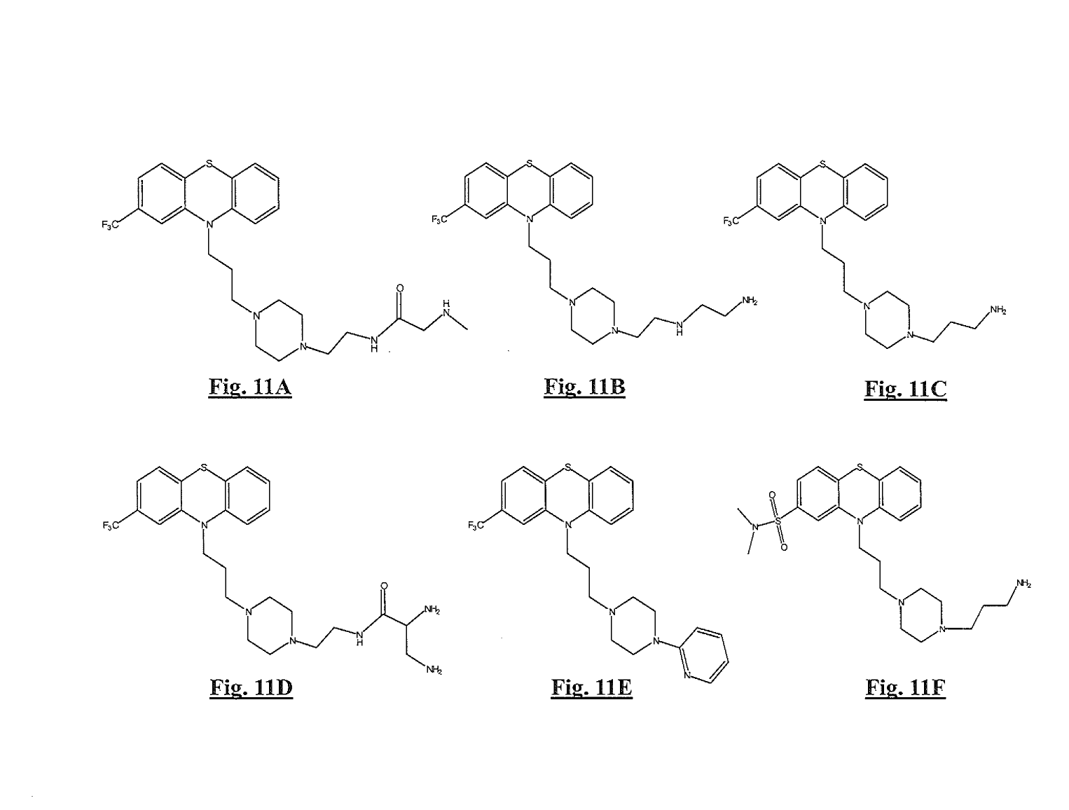

FIG. 12, comprising FIGS. 12A through 12F, is a series of images depicting the chemical structures of the following 5-HT receptor antagonists: ICI-784 (FIG. 12A), ICI-801 (FIG. 12B), ICI-802 (FIG. 12C), ICI-822 (FIG. 12D), ICI-823 (FIG. 12E), and ICI-824 (FIG. 12F).

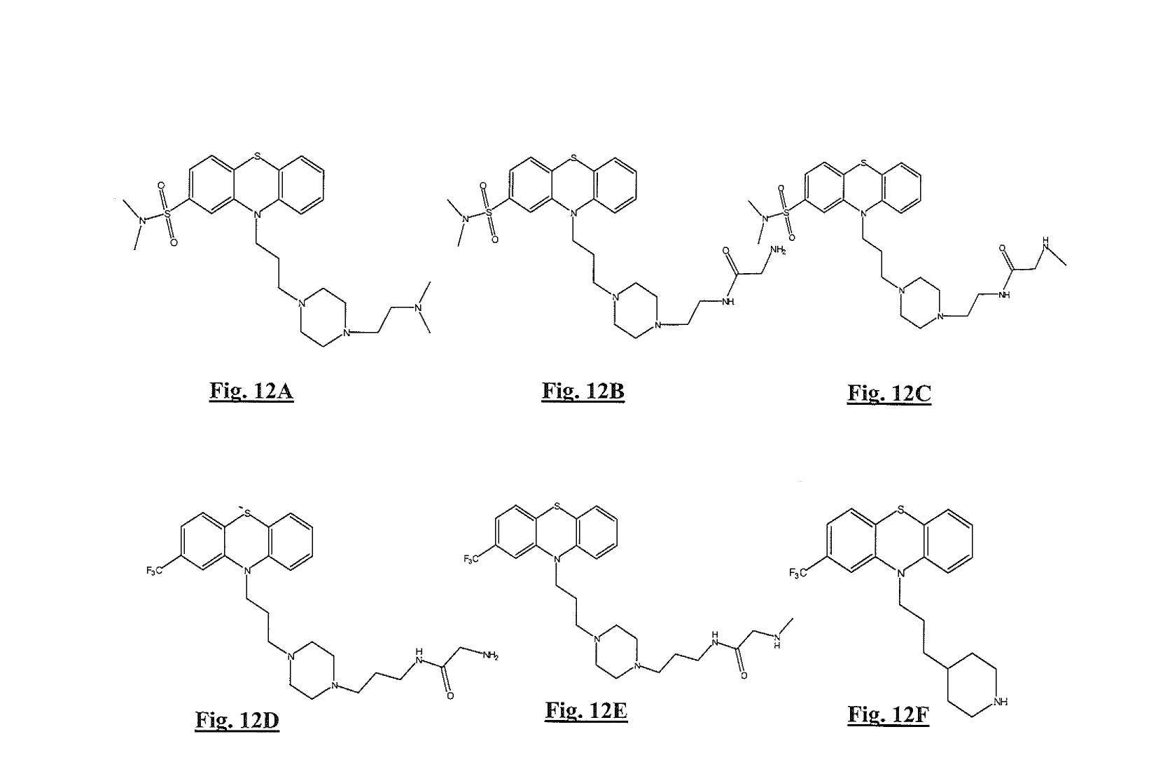

FIG. 13, comprising FIGS. 13A through 13F, is a series of images depicting the chemical structures of the following 5-HT receptor antagonists: ICI-846 (FIG. 13A), ICI-847 (FIG. 13B), ICI-848 (FIG. 13C), ICI-849 (FIG. 13D), ICI-850 (FIG. 13E), and ICI-890 (FIG. 13F).

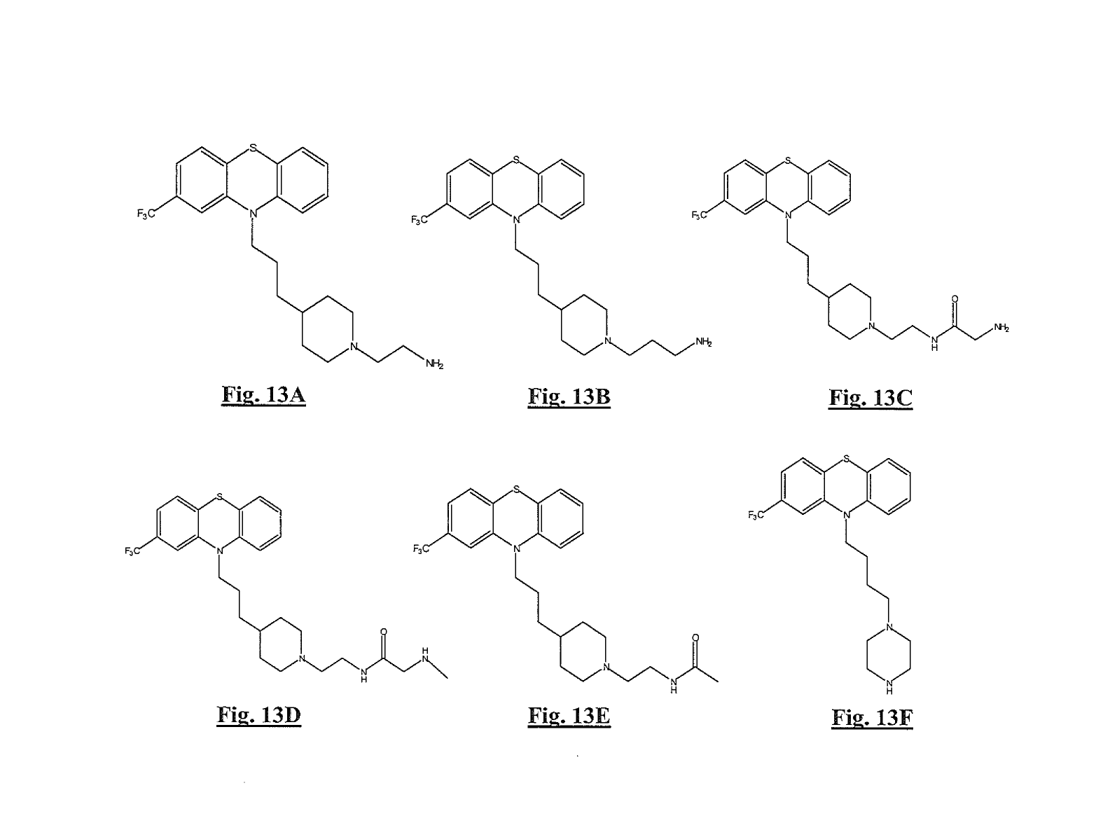

FIG. 14, comprising FIGS. 14A through 14E, is a series of images depicting the chemical structures of the following 5-HT receptor antagonists: ICI-891 (FIG. 14A), ICI-892 (FIG. 14B), ICI-893 (FIG. 14C), ICI-894 (FIG. 14D), and ICI-895 (FIG. 14E).

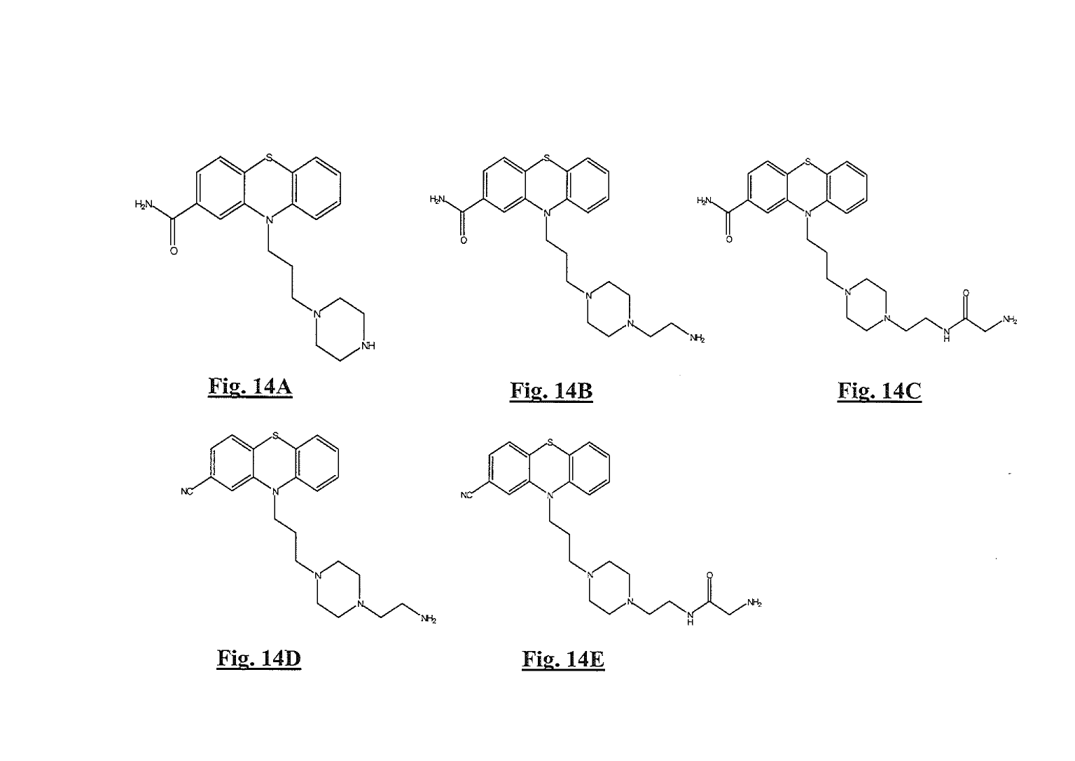

FIG. 15 is a graph depicting the results of an MTT assay demonstrating the inhibition of proliferation of RPMI-8226 cells using the indicated 5-HT receptor antagonists and the selective 5-HT1B receptor antagonist SB 216641.

FIG. 16 is a graph depicting the results of an MTT assay demonstrating the inhibition of proliferation of CCRF-CEM cells using the indicated 5-HT receptor antagonists and the selective 5-HT1B receptor antagonist SB 216641.

FIG. 17 is a graph depicting the results of an MTT assay demonstrating the inhibition of proliferation of HeLa cells using the indicated 5-HT receptor antagonists and the selective 5-HT1B receptor antagonist SB 216641.

FIG. 18 is a graph depicting the results of an MTT assay demonstrating the inhibition of proliferation of RPMI-8226 cells using the indicated 5-HT receptor antagonists and the selective 5-HT1B receptor antagonist SB 216641.

FIG. 19 is a graph depicting the results of an MTT assay demonstrating the inhibition of proliferation of CCRF-CEM cells using the indicated 5-HT receptor antagonists and the selective 5-HT1B receptor antagonist SB 216641. FIG. 30 hela

FIG. 20 is a graph depicting the results of an MTT assay demonstrating the inhibition of proliferation of RPMI-8226 cells using the indicated 5-HT receptor antagonists and the selective 5-HT1B receptor antagonist SB 216641.

FIG. 21 is a graph depicting the results of an MTT assay demonstrating the inhibition of proliferation of CCRF-CEM cells using the indicated 5-HT receptor antagonists and the selective 5-HT1B receptor antagonist SB 216641.

FIG. 22 is a graph depicting the results of an MTT assay demonstrating the inhibition of proliferation of HeLa cells using the indicated 5-HT receptor antagonists and the selective 5-HT1B receptor antagonist SB 216641.

FIG. 23A is a graph depicting the results of an MTT assay demonstrating the inhibition of proliferation of RPMI-8226 cells using the indicated 5-HT receptor antagonists and the selective 5-HT1B receptor antagonist SB 216641.

FIG. 23B, is a graph depicting the results of an MTT assay demonstrating the inhibition of proliferation of CCRF-CEM cells using the indicated 5-HT receptor antagonists and the selective 5-HT1B receptor antagonist SB 216641.

FIG. 24 is a graph depicting the results of an MTT assay demonstrating the inhibition of proliferation of HeLa cells using the indicated 5-HT receptor antagonists and the selective 5-HT1B receptor antagonist SB 216641,

FIG. 25 is a graph depicting the results of an MTT assay demonstrating the inhibition of proliferation of RPMI-8226 cells using the indicated 5-HT receptor antagonists and the selective 5-HT1B receptor antagonist SB 216641.

FIG. 26 is a graph depicting the clinical arthritis score over time for mice treated with various compounds of the invention,

FIG. 27 is a graph depicting the clinical arthritis score, with AUC calculation, for mice treated with various compounds of the invention.

FIG. 28 is a graph depicting the incidence of arthritis over time for mice treated with various compounds of the invention.

FIG. 29 is a graph depicting the clinical arthritis score over time for mice treated with various concentrations of selected compounds of the invention.

FIG. 30 is a scheme illustrating synthetic schemes for a number of compounds of the invention.

FIG. 31 is a scheme illustrating synthetic schemes for additional number of compounds of the invention.

FIG. 32 is a scheme illustrating a number of intermediates for compounds of the invention.

FIG. 33 is a scheme illustrating the synthesis of ICI-685.

FIG. 34 is a scheme illustrating one synthesis of ICI-715.

FIG. 35 is a scheme illustrating an alternate synthesis of ICI-715.

FIG. 36 is a scheme illustrating a synthesis of ICI-735.

FIG. 37 is a scheme illustrating an alternate synthesis of ICI-735.

FIG. 38 is a scheme illustrating the synthesis of ICI-824.

FIG. 39 is a scheme illustrating the synthesis of ICI-847.

FIG. 40 is a scheme illustrating the synthesis of ICI-849.

FIG. 41 is a scheme illustrating the synthesis of ICI-953.

FIG. 42 is a scheme illustrating the synthesis of ICI-954.

FIG. 43 is a scheme illustrating the synthesis of ICI-1007.

FIG. 44 is a scheme illustrating the synthesis of ICI-1008,

FIG. 45 is a scheme illustrating the synthesis of Compounds 32-36.

FIG. 46 is a scheme illustrating the synthesis of Compounds 37-38,

FIG. 47, comprising FIG. 47A through FIG. 47C, is a series of bar graphs illustrating the effect of ICI-735 on MPAP (mean pulmonary artery pressure), RVSP (right ventricular systolic pressure) and RV/BW (right ventricular/body weight ratio).

FIG. 48 is a series of bar graphs illustrating the effect of ICI-735 on MAP (mean arterial pressure) and HR (heart rate).

FIG. 49 is a bar graph illustrating the effect of ICI-735 on degree of muscularization.

FIG. 50 is a scheme illustrating metabolism of selected serotonin analogs.

### DETAILED DESCRIPTION OF THE INVENTION

The present invention includes compositions and methods for inducing cell death and/or apoptosis in activated lymphocytes. In addition, the present invention includes compositions and methods for inhibiting proliferation of activated lymphocytes. Furthermore, the present invention includes compositions and methods for preventing or treating PAH in a mammal.

As demonstrated by the data disclosed herein, the novel serotonin receptor antagonists disclosed herein inhibit proliferation and induce apoptosis in various lymphocyte cell lines, including neoplastic T cells and B cells. Thus, the present invention encompasses methods, compositions and kits for inhibiting the proliferation of lymphocytes and for inducing apoptosis in lymphocytes. The compositions and methods of the present invention are useful for treating various diseases associated with the proliferation and/or activation of lymphocytes, including, but not limited to lymphomas, myelomas, autoimmune diseases, transplant rejection, and the like. The compositions and methods of the present invention are also useful for treating PAH.

### DEFINITIONS

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

As used herein, the term “MPAP” refers to mean pulmonary artery pressure.

As used herein, the term “MAP” refers to mean arterial pressure.

As used herein, the term “RVSP” refers to right ventricular systolic pressure.

As used herein, the term “RV/BW” refers to right ventricular/body weight ratio.

As used herein, the term “HR” refers to heart rate.

As used herein, the term “SI rat” refers to a saline-injected rat.

As used herein, the term “MCT rat” refers to a MCT-injected rat.

By T cell “activation,” as the term is used herein, is meant that the T cell, when contacted with a compound, molecule, or cell capable of generating an immune response (e.g., a mitogen or antigen), detectably upregulates surface markers, such as CD25, i.e., the IL-2 receptor, initiates a phosphorylation cascade involving p561ck, causes the release of cytokines and interleukins, increases DNA synthesis which can be assessed by, among other methods, assessing the level of incorporation of ³H-thymidine into nascent DNA strands, and causes the cells to proliferate.

A “serotonin antagonist” is a composition of matter which, when administered to a mammal such as a human, detectably inhibits a biological activity attributable to the level or presence of serotonin.

A “serotonin receptor antagonist” is a composition of matter which, when administered to a mammal such as a human, detectably inhibits a biological activity attributable to the of serotonin to a serotonin receptor.

By the term “selective antagonist,” as these terms are used herein, is meant a chemical agent that has at least about a 5-fold greater affinity for the target serotonin receptor type than for any other serotonin receptor family member.

As used herein, to “alleviate” a disease means reducing the severity of one or more symptoms of the disease.

By the term “allogeneic graft,” as used herein, is meant grafting of any tissue within a species wherein there is a mismatch of an immunological marker, such as, but not limited to, the major histocompatibility complex (MHC), and/or a minor antigen.

The term “allogeneic graft response”, as used herein, means any immune response directed against non-self tissue grafted into a recipient. Grafting procedures include, but are not limited to, administering non-self cells, tissue, or organs during, e.g., bone marrow transplantation, organ transplant, and the like.

The term “apoptosis,” as used herein, means an active process, involving the activation of a preexisting cellular pathway, induced by an extracellular or intracellular signal, causing the death of the cell. In particular, the cell death involves nuclear fragmentation, chromatin condensation, and the like, in a cell with an intact membrane.

By the term “applicator,” as the term is used herein, is meant any device including, but not limited to, a hypodermic syringe, a pipette, and the like, for administering the inhibitor of serotonin interaction with a serotonin receptor (e.g., a serotonin receptor antagonist) of the invention to a mammal.

A “cell cycle process,” as used herein, means any cellular function or process associated with the cell cycle and the various phases thereof. Thus, a cell cycle process is one associated with, or which mediates or is involved in, the cell progressing through any portion of the cell cycle.

Inhibition of serotonin signaling is “deleterious” to a cell, as the term is used herein, where the inhibition mediates a detectable decrease in the viability of the cell. Cell viability can be assessed using standard methods that are well-known in the art, including, but not limited to, assessing the level of biomolecular synthesis (e.g., protein synthesis, nucleic acid synthesis, and the like), trypan blue exclusion, MTT reduction, uptake of propidium iodide, exposure of phosphatidylserine on the cell surface, DNA fragmentation and/or ladder formation, and the like.

A “disease” is a state of health of an animal wherein the animal cannot maintain homeostasis, and wherein if the disease is not ameliorated, then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

By the term “does not substantially cross the blood-brain barrier”, as used herein, means that the inhibitor does not detectably cross the blood-brain barrier as assessed using standard assays such as those disclosed herein, known in the art, or such assays as are developed in the future to determine the permeability of a compound across the blood-brain barrier. Such assays include, but are not limited to, assessing the neuro-psychotropic effects of the compound when administered to an animal. Further, the assays encompass, among other things, assessing the concentration of the compound beyond the barrier, or an art-recognized model of the blood-brain barrier, over time to determine the permeability of the compound through the barrier.

It would be understood by the artisan that an inhibitor can be ab initio impermeable and not cross the blood-brain barrier at a detectable level. Further, it would be understood that an inhibitor of interest can be modified, using techniques well-known in the art, such that it does not detectably cross the blood-brain barrier, or crosses it at a detectably lower level that it did before it was modified. In both instances, whether it loses its ability to cross the blood-brain barrier at a detectable level or loses the ability to cross it at a lower level than before it was modified, the compound is considered to “not substantially cross the blood-brain barrier” for purposes of this section.

By the term “effective amount”, as used herein, is meant an amount of an inhibitor that is sufficient to mediate a detectable decrease in transmission of serotonin signaling via a serotonin receptor on a cell. Transmission of a serotonin signal can be assessed using standard methods well-known in the art, such as, but not limited to, those described elsewhere herein, including, for example, assessing the level of binding of serotonin with a receptor and/or assessing the level of activation of a cell.

The skilled artisan would understand that the amount varies and can be readily determined based on a number of factors such as the disease or condition being treated, the age and health and physical condition of the mammal being treated, the severity of the disease, the particular compound being administered, and the like. Generally, the dosage will be set between 1 mg/kg and 25 mg/kg. In one embodiment, the drug is administered through intravenous bolus injection. This type of bolus administration can be used to ensure that all of the immunologically relevant cells encounter sufficient quantity of the drug in order to block their receptor-mediated signals. However, the invention is not limited to this method of administration.

As used herein, the term “pharmaceutically acceptable carrier” means a chemical composition with which the active ingredient may be combined and which, following the combination, can be used to administer the active ingredient to a subject.

As used herein, the term “physiologically acceptable” ester or salt means an ester or salt form of the active ingredient which is compatible with any other ingredients of the pharmaceutical composition, which is not deleterious to the subject to which the composition is to be administered.

By the term “immune reaction,” as used herein, is meant the detectable result of stimulating and/or activating an immune cell.

“Immune response,” as the term is used herein, means a process that results in the activation and/or invocation of an effector function in either the T cells, B cells, natural killer (NK) cells, and/or antigen-presenting cells (APCs). Thus, an immune response, as would be understood by the skilled artisan, includes, but is not limited to, any detectable antigen-specific or allogeneic activation of a helper T cell or cytotoxic T cell response, production of antibodies, T cell-mediated activation of allergic reactions, and the like.

“Immune cell,” as the term is used herein, means any cell involved in the mounting of an immune response. Such cells include, but are not limited to, T cells, B cells, NK cells, antigen-presenting cells, and the like.

“Instructional material,” as that term is used herein, includes a publication, a recording, a diagram, or any other medium of expression which can be used to communicate the usefulness of the nucleic acid, peptide, and/or compound of the invention in the kit for effecting alleviating or treating the various diseases or disorders recited herein. Optionally, or alternately, the instructional material may describe one or more methods of alleviating the diseases or disorders in a cell or a tissue of a mammal. The instructional material of the kit may, for example, be affixed to a container that contains the nucleic acid, peptide, and/or compound of the invention or be shipped together with a container which contains the nucleic acid, peptide, and/or compound. Alternatively, the instructional material may be shipped separately from the container with the intention that the recipient uses the instructional material and the compound cooperatively.

By the term “serotonin family receptor” is meant any receptor which can be classified as a serotonin, adrenergic, histamine, melatonin, or dopaminergic receptor. That is, the receptor specifically binds with any of these molecules and does not significantly bind with other molecules in a sample.

A “serotonin receptor” includes a polypeptide that specifically binds with serotonin.

“Serotonin signal,” as the term is used herein, means a change in the balance of any intracellular biochemical pathway as a result of a receptor-mediated interaction with serotonin, a specific drug interaction with any serotonin-specific receptor, or both, that results in the change.

Similarly, “activation of a serotonin” receptor, as used herein, means that binding of serotonin with a serotonin receptor on a cell induces the typical cascade of intra and extracellular events associated with such binding.

A “receptor” is a compound that specifically binds with a ligand.

By the term “specifically binds,” as used herein, is meant a receptor which recognizes and binds serotonin family molecules present in a sample (i.e., dopaminergic proteins, adrenergic protein, histamines, melatonin, and serotonin), but does not substantially recognize or bind other molecules in the sample.

To “treat” a disease as the term is used herein, means to reduce the frequency of the disease or disorder reducing the frequency with which a symptom of the one or more symptoms disease or disorder is experienced by an animal.

### DESCRIPTION

The present invention includes methods, compositions and kits for treating diseases and conditions associated with the proliferation of activated lymphocytes and the diseases resulting from the activation of lymphocytes. The present invention encompasses methods for inhibiting and killing activated lymphocytes, compositions that inhibit and/or kill activated lymphocytes, compositions that inhibit the proliferation of activated lymphocytes, and kits for using the methods and compositions of the invention.

The compositions of the present invention include 5-HT receptor antagonists having the chemical formulae disclosed elsewhere herein. The compositions disclosed herein further comprise combinations of these 5-HT receptor antagonists with additional compositions for inhibiting and/or killing activated lymphocytes. As demonstrated by the data disclosed herein, the compositions of the present invention inhibit and/or kill activated lymphocytes by, among other things, inducing apoptosis and cell death in activated lymphocytes. In addition, the compounds of the present invention inhibit proliferation of lymphocytes, such as T cells and B cells, and are therefore useful in the treatment of diseases where activated and/or proliferating lymphocytes cause pathology. Such diseases include, but are not limited to, lymphomas, myelomas, autoimmune diseases, and transplant rejection.

The methods of the present invention encompass methods of inhibiting and/or killing an activated lymphocyte, and methods of inhibiting the proliferation of a lymphocyte. This is because, as demonstrated by the data disclosed herein, the methods of the invention cause a dose and time dependent inhibition of proliferating lymphocytes, as well as dose and time dependent apoptosis in lymphocytes. The methods of the present invention further comprise methods of treating a patient suffering from a disease associated with an activated lymphocyte. Such diseases are known in the art and are disclosed elsewhere herein. The methods of the invention are based, in part, on the novel finding that 5-HT receptor antagonists, such as those disclosed herein, are useful in inhibiting and/or killing activated lymphocytes.

The methods of the present invention encompass methods of preventing or treating PAH in a mammal.

### Compositions

The compositions of the present invention include a composition of formula I, II or III, as well as the compositions disclosed below. The present invention comprises compositions for inhibiting and/or killing activated lymphocytes, for inhibiting proliferation in lymphocytes, and for treating diseases associated with such lymphocytes. One embodiment of the present invention includes compositions which, as demonstrated by the data disclosed herein, induce cell death and apoptosis in various activated lymphocytes, including T cells and B cells.

As demonstrated by the data disclosed herein, 5-HT receptor antagonists having the structure of formula I, II or III are useful in the present invention for inhibiting the proliferation of lymphocytes, such as T cells and B cells, and for inducing apoptosis and/or cell death in lymphocytes. Thus, the compounds of the present invention is useful for treating, among other things, lymphomas, myelomas, autoimmune diseases, transplant rejection, and the like. The compounds of the present invention are also useful for preventing or treating PAH.

The present invention includes a composition comprising a compound of formula I, or a pharmaceutically acceptable salt thereof:

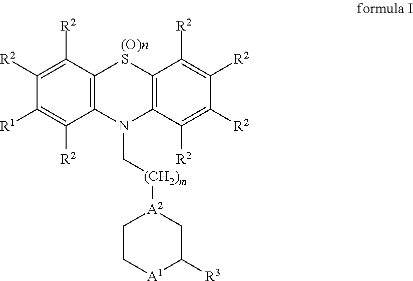

wherein:

R¹ is independently selected at each occurrence from hydrogen, halogen, (C₁-C₆)alkyl; (C₁-C₆)alkenyl; (C₁-C₆)alkoxy; OH; NO₂; C≡N; C(═O)OR⁷; C(═O)NR⁷₂; NR⁷₂; NR⁷C(═O)(C₁-C₆)alkyl; NR⁷C(═O)O(C₁-C₆)alkyl; NR⁷C(═O)NR⁷₂; NR⁷SO₂(C₁-C₆)alkyl; SO₂NR⁷₂; OC(═O)(C₁-C₆)alkyl; O(C₂-C₆)alkylene-NR⁷₂; (C₂-C₆)alkylene-OR⁷; and (C₁-C₃)perfluoroalkyl;

R² is independently selected at each occurrence from hydrogen, halogen, (C₁-C₆)alkyl; (C₁-C₆)alkenyl; (C₁-C₆)alkoxy; OH; NO₂; C≡N; C(═O)OR⁷; C(═O)NR⁷₂; NR⁷₂; NR⁷C(═O)(C₁-C₆)alkyl; NR⁷C(═O)O(C₁-C₆)alkyl; NR⁷C(═O)NR⁷₂; NR⁷SO₂(C₁-C₆)alkyl; SO₂NR⁷₂; OC(═O)(C₁-C₆)alkyl; O(C₂-C₆)alkylene-NR⁷₂; (C₂-C₆)alkylene-OR⁷; and (C₁-C₃)perfluoroalkyl;

R³ is hydrogen, C(═O)OR⁷, or C(═O)NR⁷₂;

A¹ is CH₂ or NR⁴;

A² is CH or N; provided that if A¹ is CH₂, then A² is N, and if A² is CH, then A¹ is NR⁴;

R⁴ is H, substituted or unsubstituted aryl, substituted or unsubstituted heteroaryl; (C₁-C₆)alkyl; (CH₂)ₚOR⁷; (CH₂)ₚNR⁷₂; (CH₂)ₚNR⁷C(O)R⁵; (CH₂)ₚO(CH₂)ₚOR⁷; (CH₂)ₚO(CH₂)ₚNR⁷₂; (CH₂)ₚNR⁴(CH₂)ₚNR⁷₂; (CH₂)ₚO(CH₂)ₚNHC(O)R⁵; (CH₂)ₚNR⁷(CH₂)ₚNHC(O)R⁵; (CH₂)_{q}C(═O)OR⁷; (CH₂)_{q}C(═O)NR⁷₂; (CH₂)ₚO(CH₂)_{q}C(═O)OR⁷; (CH₂)ₚO(CH₂)_{q}C(═O)NR⁷₂; (CH₂)ₚNR⁷(CH₂)_{q}C(═O)OR⁷; (CH₂)ₚNR⁷(CH₂)_{q}C(═O)NR⁷₂; or

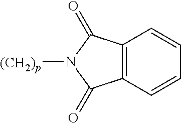

R⁵ is H, (C₁-C₆)alkyl; CR⁸R⁹R¹⁰; NR⁷C(═O)(C₁-C₆)alkyl; NR⁷C(═O)O(C₁-C₆)alkyl; NR⁷C(═O)NR⁷₂; CH(R⁶)NR⁷₂; CH(R⁶)NR⁷C(═O)(C₁-C₆)alkyl; or CH(R⁶)NR⁷C(═O)O(C₁-C₆)alkyl.

R⁶ is H, (C₁-C₆)alkyl; (C₂-C₆)alkylene-OR⁷; (CH₂)_{q}C(═O)OR⁷; or (CH₂)_{q}C(═O)NR⁷₂;

each occurrence of R⁷ and R¹⁰ is independently selected from the group consisting of hydrogen, (C₁-C₆)cycloalkyl and (C₁-C₆)alkyl;

each occurrence of R⁸ and R⁹ is independently selected from the group consisting of (C₁-C₆)cycloalkyl and (C₁-C₆)alkyl;

m is independently at each occurrence 1, 2, or 3;

n is 0, 1, or 2;

p is independently at each occurrence 2 or 3; and

q is independently at each occurrence 1 or 2;

wherein the substituents for the substituted aryl and substituted heterocyclic groups comprising or included within R⁴ are independently selected from the group consisting of halogen, (C₁-C₆)alkyl; (C₁-C₆)alkenyl; (C₁-C₆)alkoxy; OH; NO₂; C-=═N; C(═O)OR⁷; C(═O)NR⁷₂; NR⁷₂; NR⁷C(═O)(C₁-C₆)alkyl; NR⁷C(═O)O(C₁-C₆)alkyl; NR⁷C(═O)NR⁷₂; NR⁷SO₂(C₁-C₆)alkyl; SO₂NR⁷₂; OC(═O)(C₁-C₆)alkyl; O(C₂-C₆)alkylene-NR⁷₂; (C₂-C₆)alkylene-OR⁷; and (C₁-C₃)perfluoroalkyl.

In one preferred embodiment, R¹ is hydrogen, halogen, (C₁-C₆)alkyl, preferably methyl, C≡N, C(═O)NR⁷₂, preferably C(═O)NH₂, SO₂NR⁷₂, preferably SO₂NMe₂, or (C₁-C₃)perfluoroalkyl, preferably CF₃. In one preferred embodiment, R¹ is hydrogen, C≡N, or CF₃.

In one preferred embodiment, one or fewer occurrences of R² are other than hydrogen, and in a most preferred embodiment, each occurrence of R² is hydrogen. In one preferred embodiment, R³ is hydrogen. In one preferred embodiment, A¹ is NR⁴. In one preferred embodiment, A² is N. In one more preferred embodiment, A¹ is NR⁴ and A² is N.

In one preferred embodiment, R⁴ is H, (CH₂)ₚNR⁷₂, preferably CH₂CH₂NH₂ or CH₂CH₂CH₂NH₂, (CH₂)ₚNR⁷C(O)R⁵, preferably CH₂CH₂NC(O)R⁵, more preferably CH₂CH₂NHC(O)Me, CH₂CH₂NHC(O)CH₂NH₂, or CH₂CH₂NHC(O)CH₂NMe. In one preferred embodiment, R⁵ is (C₁-C₆)alkyl; or CH(R⁶)NR⁷₂, preferably CH(R⁶)NH₂ or NHMe. In one preferred embodiment, R⁶ is H. In one preferred embodiment, m is 2. In one preferred embodiment, n is 0. In one preferred embodiment, p is 2. In one preferred embodiment, q is 1.

In one embodiment, R¹ is hydrogen, halogen, (C₁-C₆)alkyl, methyl, C≡N, C(═O)NR⁷₂, C(═O)NH₂, SO₂NR⁷₂, SO₂NMe₂, (C₁-C₃)perfluoroalkyl, or CF₃. In another embodiment, each occurrence of R² is hydrogen. In yet another embodiment, R³ is hydrogen. In yet another embodiment, A¹ is NR⁴. In yet another embodiment, A² is N.

In one embodiment, R⁴ is H, (CH₂)ₚNR⁷₂, CH₂CH₂NH₂, CH₂CH₂CH₂NH₂, (CH₂)ₚNR⁷C(O)R⁵, CH₂CH₂NHC(O)R⁵, CH₂CH₂NHC(O)Me, CH₂CH₂NHC(O)CH₂NH₂, or CH₂CH₂NHC(O)CH₂NMe. In another embodiment, R⁴ is (CH₂)ₚNR⁷C(O)R⁵. In yet another embodiment, R⁴ is (CH₂)ₚNHC(O)R⁵.

In one embodiment, R⁵ is (C₁-C₆)alkyl, CH(R⁶)NR⁷₂, or CH(R⁶)NH₂ or NHMe. In another embodiment, R⁵ is H or CR⁸R⁹R¹⁰.

In one embodiment, R⁶ is H. In yet another embodiment, m is 2, n is 0, p is 2, and q is 1.

The present invention also includes a composition comprising a compound of formula II, or a pharmaceutically acceptable salt thereof:

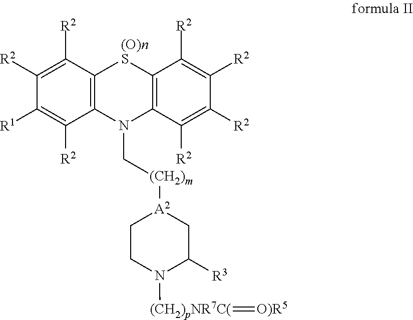

wherein:

each occurrence of R¹ and R² is independently selected from the group consisting of hydrogen, halogen, (C₁-C₆)alkyl; (C₁-C₆)alkenyl; (C₁-C₆)alkoxy; OH; NO₂; C≡N; C(═O)OR⁷; C(═O)NR⁷₂; NR⁷₂; NR⁷C(═O)(C₁-C₆)alkyl; NR⁷C(═O)O(C₁-C₆)alkyl; NR⁷C(═O)NR⁷₂; NR⁷SO₂(C₁-C₆)alkyl; SO₂NR⁷₂; OC(═O)(C₁-C₆)alkyl; O(C₂-C₆)alkylene-NR⁷₂; (C₂-C₆)alkylene-OR⁷; and (C₁-C₃)perfluoroalkyl;

R³ is hydrogen, C(═O)OR⁷, or C(═O)NR⁷₂;

A² is CH or N;

R⁵ is H or CR⁸R⁹R¹⁰;

each occurrence of R⁷ and R¹⁰ is independently selected from the group consisting of hydrogen, (C₁-C₆)cycloalkyl and (C₁-C₆)alkyl;

each occurrence of R⁸ and R⁹ is independently selected from the group consisting of (C₁-C₆)cycloalkyl and (C₁-C₆)alkyl; or R⁸ and R⁹ are bound to the same carbon atom and linked as to form a divalent group selected from the group consisting of ethane-1,2-diyl, propane-1,3-diyl, butane-1,4-diyl, pentane-1,5-diyl, hexane-1,6-diyl and heptane-17-diyl; wherein said bivalent group is optionally substituted with at least one (C₁-C₆)alkyl group;

m is independently at each occurrence 1, 2, or 3;

n is 0, 1, or 2;

p is independently at each occurrence 2 or 3; and

q is independently at each occurrence 1 or 2.

In one embodiment, the compound of formula II is N-(2-(4-(3-(2-(trifluoromethyl)-10H-phenothiazin-10-yl)propyl)piperazin-1-yl)ethyl)pivalamide (Compound 35b) or a salt thereof.

The present invention further includes a composition comprising a compound of formula III, or a pharmaceutically acceptable salt thereof:

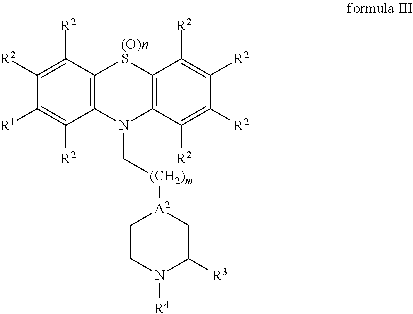

wherein:

each occurrence of R¹ and R² is independently selected from the group consisting of hydrogen, halogen, (C₁-C₆)alkyl; (C₁-C₆)alkenyl; (C₁-C₆)alkoxy; OH; NO₂; C≡N; C(═O)OR⁵; C(═O)NR⁵₂; NR⁵₂; NR⁵C(═O)(C₁-C₆)alkyl; NR⁵C(═O)O(C₁-C₆)alkyl; NR⁵C(═O)NR⁵₂; NR⁵SO₂(C₁-C₆)alkyl; SO₂NR⁵₂; OC(═O)(C₁-C₆)alkyl; O(C₂-C₆)alkylene-NR⁵₂; (C₂-C₆)alkylene-OR⁵; and (C₁-C₃)perfluoroalkyl;

R³ is hydrogen, C(═O)OR⁵, or C(═O)N(R⁵)₂;

A² is CH or N;

R⁴ is —(CR⁵₂)ₚN(R⁵)C(═O)—CR⁶R⁷R⁸; —(CR⁵₂)ₚO(CR⁵₂)ₚN(R⁵)C(═O)—CR⁶R⁷R⁸; —(CR⁵₂)ₚN(R⁵)(CR⁵₂)ₚN(R⁵)C(═O)—CR⁶R⁷R⁸; or —(CR⁵₂)ₚN(R⁵)C(═O)(CR⁵₂)ₚN(R⁵)C(═O)—CR⁶R⁷R⁸;

each occurrence of R⁵ and R⁶ is independently selected from the group consisting of hydrogen, (C₁-C₆)alkyl and (C₁-C₆)cycloalkyl;

R⁷ is (C₁-C₆)alkyl or (C₁-C₆)cycloalkyl; or R⁶ and R⁷ are bound to the same carbon atom and linked as to form a divalent group selected from the group consisting of ethane-1,2-diyl, propane-1,3-diyl, butane-1,4-diyl, pentane-1,5-diyl, hexane-1,6-diyl and heptane-17-diyl; wherein said bivalent group is optionally substituted with at least one (C₁-C₆)alkyl group;

R⁸ is (C₁-C₆)alkyl, —N(R⁵)C(═O)R⁵, or —N(R⁵)S(═O)₂R⁷;

m is independently at each occurrence 1, 2, or 3;

n is 0, 1, or 2; and,

p is independently at each occurrence 1, 2 or 3.

In one embodiment, R¹ is hydrogen, halogen, (C₁-C₆)alkyl, methyl, C≡N, C(═O)NR⁷₂, C(═O)NH₂, SO₂NR⁷₂, SO₂NMe₂, (C₁-C₃)perfluoroalkyl, or CF₃. In another embodiment, each occurrence of R² is hydrogen. In yet another embodiment, R³ is hydrogen.

In one embodiment, A² is N. In another embodiment, R⁴ is —(CR⁵₂)ₚN(R⁵)C(═O)—CR⁶R⁷R⁸. In yet another embodiment, m is 2 or 3. In yet another embodiment, n is 0. In yet another embodiment, p is 2. In yet another embodiment, R⁸ is (C₁-C₆)alkyl or —N(R⁵)(C═O)R⁵.

In one embodiment, the compound of formula III is selected from the group consisting of 2-amino-2-methyl-N-(2-(4-(3-(2-(trifluoromethyl)-10H-phenothiazin-10-yl)propyl)piperazin-1-yl)ethyl)propanamide (Compound 36a), 2-formamido-N-(2-(4-(3-(2-(trifluoromethyl)-10H-phenothiazin-10-yl)propyl)piperazin-1-yl)ethyl)acetamide (Compound 37b), a salt thereof, and mixtures thereof.

In one embodiment, the compound useful within the methods of the invention is selected from the group consisting of ICI-681, ICI-682, ICI-683, ICI-684, ICI-685, ICI-686, ICI-687, ICI-696, ICI-697, ICI-712, ICI-713, and ICI-714, ICI-715, ICI-726, ICI-727, ICI-728, ICI-734, ICI-735, ICI-737, ICI-738, ICI-746, ICI-747, ICI-748, ICI-749, ICI-758, ICI-759, ICI-760, ICI-761, ICI-763, ICI-783, ICI-784, ICI-801, ICI-802, ICI-822, ICI-823, ICI-824, ICI-846, ICI-847, ICI-848, ICI-849, ICI-850, ICI-890, ICI-891, ICI-892, ICI-893, ICI-894, ICI-895, 2-amino-2-methyl-N-(2-(4-(3-(2-(trifluoromethyl)-10H-phenothiazin-10-yl)propyl)piperazin-1-yl)ethyl)propanamide (Compound 36a); N-(2-(4-(3-(2-(trifluoromethyl)-101′-phenothiazin-10-yl)propyl)-piperazin-1-yl)ethyl)pivalamide (Compound 35b), 2-formamido-N-(2-(4-(3-(2-(trifluoromethyl)-10H-phenothiazin-10-yl)propyl)piperazin-1-yl)ethyl)acetamide (Compound 37b), a salt thereof and combinations thereof.

In the definitions of each of the compounds of formula I, II or III above, the following definitions apply in some embodiments.

The term “alkyl”, by itself or as part of another substituent means, unless otherwise stated, a straight, branched or cyclic chain hydrocarbon having the number of carbon atoms designated (i.e. C₁-C₆ means one to six carbons) and includes straight, branched chain or cyclic groups. Examples include; methyl, ethyl, propyl, isopropyl, butyl, isobutyl, tertbutyl, pentyl, neopentyl, hexyl, cyclohexyl and cyclopropylmethyl. Most preferred is (C₁-C₃)alkyl, particularly ethyl, methyl and isopropyl.

The term “alkenyl” employed alone or in combination with other terms, means, unless otherwise stated, a stable monounsaturated or di-unsaturated straight chain, branched chain or cyclic hydrocarbon group having the stated number of carbon atoms. Examples include vinyl, propenyl crotyl, isopentenyl, butadienyl, 1,3-pentadienyl, 1,4-pentadienyl, cyclopentenyl, cyclopentadienyl and the higher homologs and isomers. A functional group representing an alkene is exemplified by CH═CHCH₂.

The term “alkylene”, by itself or as part of another substituent means, unless otherwise stated, a divalent straight, branched or cyclic chain hydrocarbon. The term “alkoxy” employed alone or in combination with other terms means, unless otherwise stated, an alkyl group having the designated number of carbon atoms, as defined above, connected to the rest of the molecule via an oxygen atom, such as, for example, methoxy, ethoxy, 1-propoxy, 2-propoxy (isopropoxy) and the higher homologs and isomers. Preferred are (C₁-C₃)alkoxy, particularly ethoxy and methoxy.

The term “aryl”, employed alone or in combination with other terms, means, unless otherwise stated, a carbocyclic aromatic system containing one or more rings (typically one, two or three rings) wherein such rings may be attached together in a pendent manner, such as a biphenyl, or may be fused, such as naphthalene. Examples include phenyl; anthracyl; and naphthyl. Preferred are phenyl and naphthyl, most preferred is phenyl.

The term “heteroaryl” refers to a heterocycle having aromatic character. A polycyclic heteroaryl may include one or more rings which are partially saturated. Examples include tetrahydroquinoline and 2,3-dihydrobenzofuryl. For compounds of formula I, II or III, the attachment point is understood to be on an atom which is part of an aromatic monocyclic ring or a ring component of a polycyclic aromatic which is itself an aromatic ring.

Examples of heteroaryl groups include: pyridyl, pyrazinyl, pyrimidinyl, particularly 2 and 4 pyrimidinyl, pyridazinyl, thienyl, furyl, pyrrolyl, particularly 2-pyrrolyl, imidazolyl, thiazolyl, oxazolyl, pyrazolyl, particularly 3- and 5-pyrazolyl, isothiazolyl, 1,2,3-triazolyl, 1,2,4-triazolyl, 1,3,4-triazolyl, tetrazolyl, 1,2,3-thiadiazolyl, 1,2,3-oxadiazolyl, 1,3,4-thiadiazolyl and 1,3,4-oxadiazolyl.

Examples of polycyclic heterocycles include: indolyl, particularly 3-, 4-, 5-, 6- and 7-indolyl, indolinyl, quinolyl, tetrahydroquinolyl, isoquinolyl, particularly 1- and 5-isoquinolyl, 1,2,3,4-tetrahydroisoquinolyl, cinnolinyl, quinoxalinyl, particularly 1- and 5-quinoxalinyl, quinazolinyl, phthalazinyl, 1,8-naphthyridinyl, 1,4-benzodioxanyl, coumarin, dihydrocoumarin, benzofuryl, particularly 3, 4, 1, 5 naphthyridinyl, 5-, 6- and 7-benzofuryl, 2,3-dihydrobenzofuryl, 1,2-benzisoxazolyl, benzothienyl, particularly 3-, 4-, 5-, 6-, and 7-benzothienyl, benzoxazolyl, benzthiazolyl, particularly 2-benzothiazolyl and 5-benzothiazolyl, purinyl, benzimidazolyl, particularly 2-benzimidazolyl, benztriazolyl, thioxanthinyl, carbazolyl, carbolinyl, acridinyl, pyrrolizidinyl, and quinolizidinyl. The aforementioned listing of heteroaryl moieties is intended to be representative and not limiting.

The term halogen means, unless otherwise stated, a fluorine, chlorine, bromine, or iodine atom, preferably, fluorine, chlorine, or bromine, more preferably, fluorine or chlorine.

The term “(Cₓ-C_{y})perfluoroalkyl,” wherein x<y, means an alkyl group with a minimum of x carbon atoms and a maximum of y carbon atoms, wherein all hydrogen atoms are replaced by fluorine atoms. Preferred is —CF₃.

The compounds of formula I, II or III can be prepared by a person skilled in the art of synthetic organic chemistry. The person skilled in the art knows how to select and implement appropriate synthetic routes. Suitable synthetic methods may be identified by reference to the literature describing synthesis of analogous compounds, and then performing the synthesis of the desired compound following the route used for the analogous compounds, modifying the starting materials, reagents, and reaction conditions as appropriate to synthesizing any particular desired compounds. In addition, reference may be made to sources such as Comprehensive Organic Synthesis, Ed. B. M. Trost and I. Fleming (Pergamon Press 1991), Comprehensive Organic Functional Group Transformations, Ed. A. R. Katritzky, O. Meth Cohn, and C. W. Rees (Pergamon Press, 1996), Comprehensive Organic Functional Group Transformations II, Ed. A. R. Katritzky and R. J. K. Taylor (Editor) (Elsevier, 2nd Edition, 2004), Comprehensive Heterocyclic Chemistry, Ed. A. R. Katritzky and C. W. Rees (Pergamon Press, 1984), and Comprehensive Heterocyclic Chemistry H, Ed. A. R. Katritzky, C. W. Rees, and E. F. V. Scriven (Pergamon Press, 1996), the entire disclosures of which are incorporated herein by reference.

It will be understood that when compounds of formula I, II or III contain one or more chiral centers, the compounds may exist in and may be isolated as pure enantiomeric or diastereomeric forms or as racemic mixtures. The present invention therefore includes any possible enantiomers, diastereomers, racemates or mixtures thereof of the compounds of the invention which are efficacious in the treatment of diseases associated with activated and/or proliferating lymphocytes, including, but not limited to, lymphomas, myelomas, autoimmune diseases, and transplant rejection.

The isomers resulting from the presence of a chiral center comprise a pair of non superimposable isomers that are called “enantiomers.” Single enantiomers of a pure compound are optically active, i.e., they are capable of rotating the plane of plane polarized light.

The present invention is meant to encompass diastereomers as well as their racemic and resolved, diastereomerically and enantiomerically pure forms and salts thereof. Diastereomeric pairs may be resolved by known separation techniques including normal and reverse phase chromatography, and crystallization.

By “isolated optical isomer” means a compound which has been substantially purified from the corresponding optical isomer(s) of the same formula. Preferably, the isolated isomer is at least about 80%, more preferably at least 90% pure, even more preferably at least 98% pure, most preferably at least about 99% pure, by weight.

Isolated optical isomers may be purified from racemic mixtures by well known chiral separation techniques. According to one such method, a racemic mixture of a compound having the structure of formula I, II or III, or a chiral intermediate thereof, is separated into 99% wt. % pure optical isomers by HPLC using a suitable chiral column, such as a member of the series of DAICEL® CHIRALPAK® family of columns (Daicel Chemical Industries, Ltd., Tokyo, Japan). The column is operated according to the manufacturer's instructions.

The present invention further comprises compositions for inhibiting and/or killing activated lymphocytes, for inhibiting proliferating lymphocytes, for treating diseases associated with such lymphocytes, or for treating PAH in a mammal. One embodiment of the present invention includes compositions which, as demonstrated by the data disclosed herein, induce cell death and apoptosis in a variety of activated lymphocytes, including T cells and B cells, or treat PAH in a mammal. The compositions of the present invention include the compositions disclosed below.

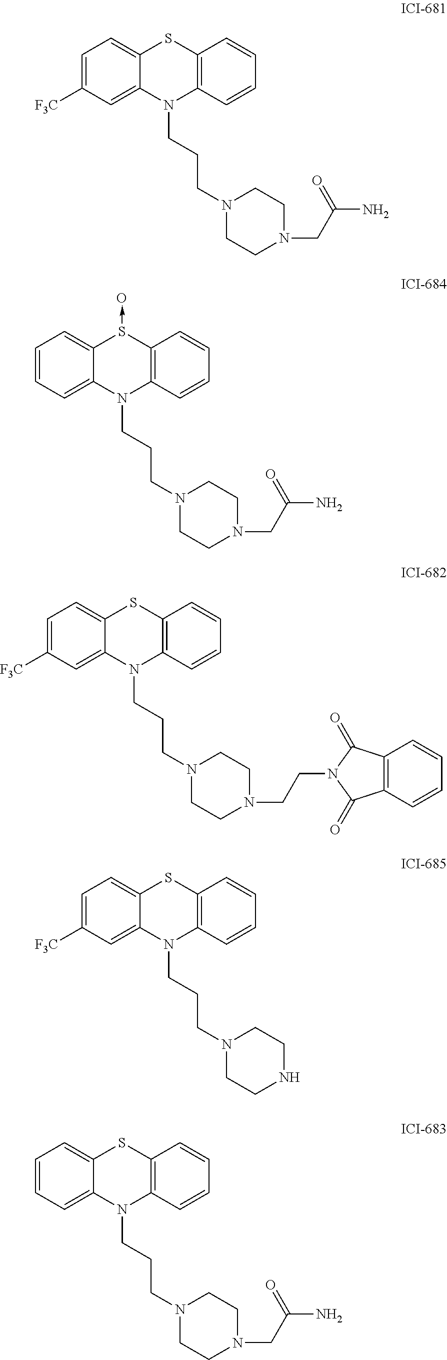

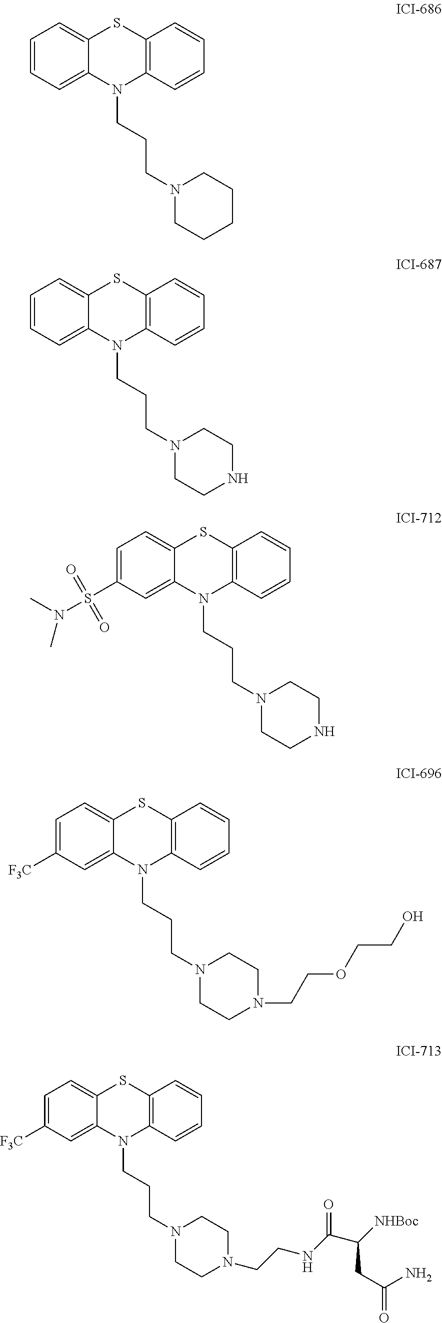

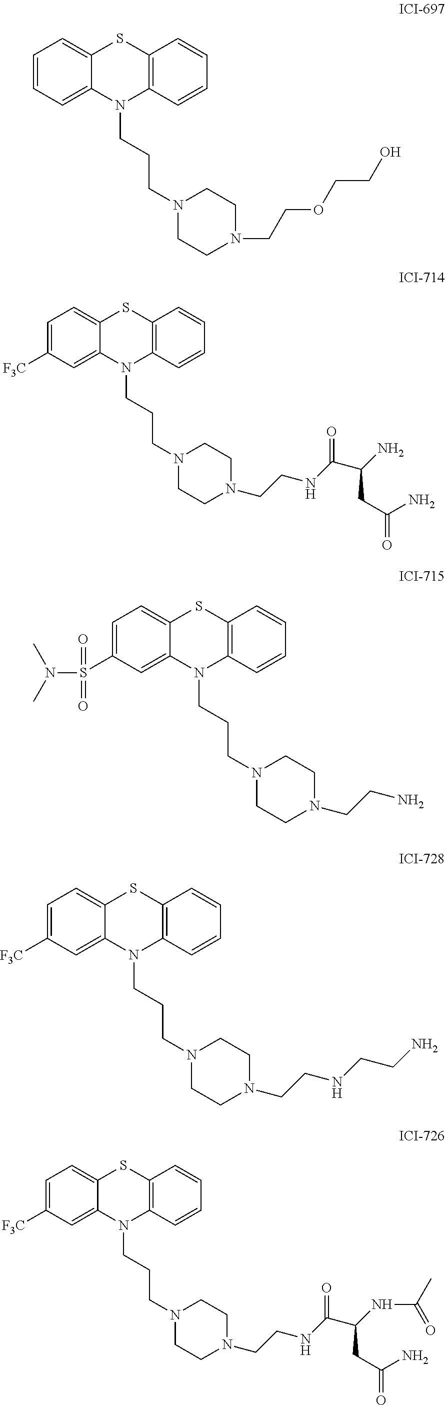

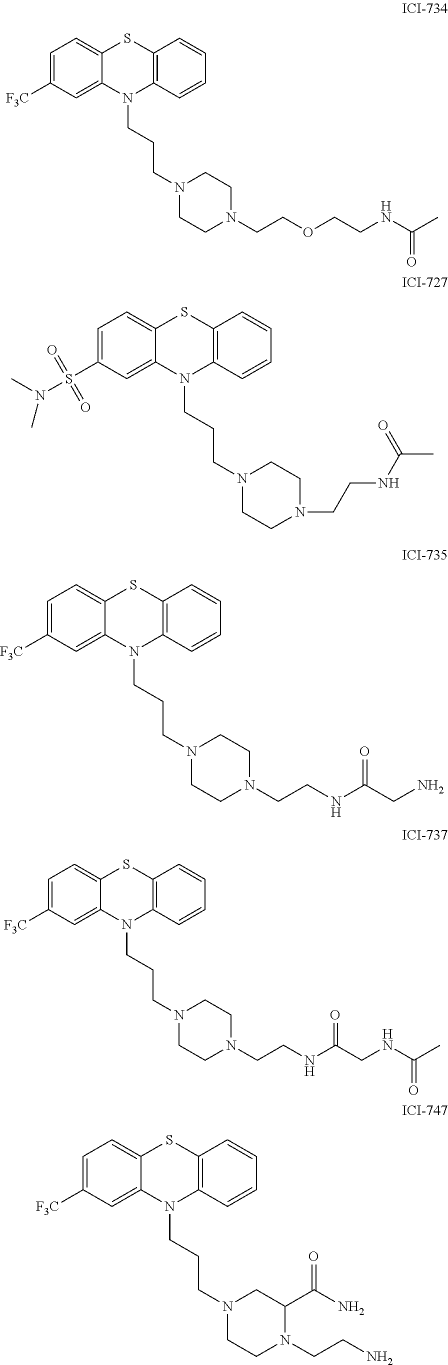

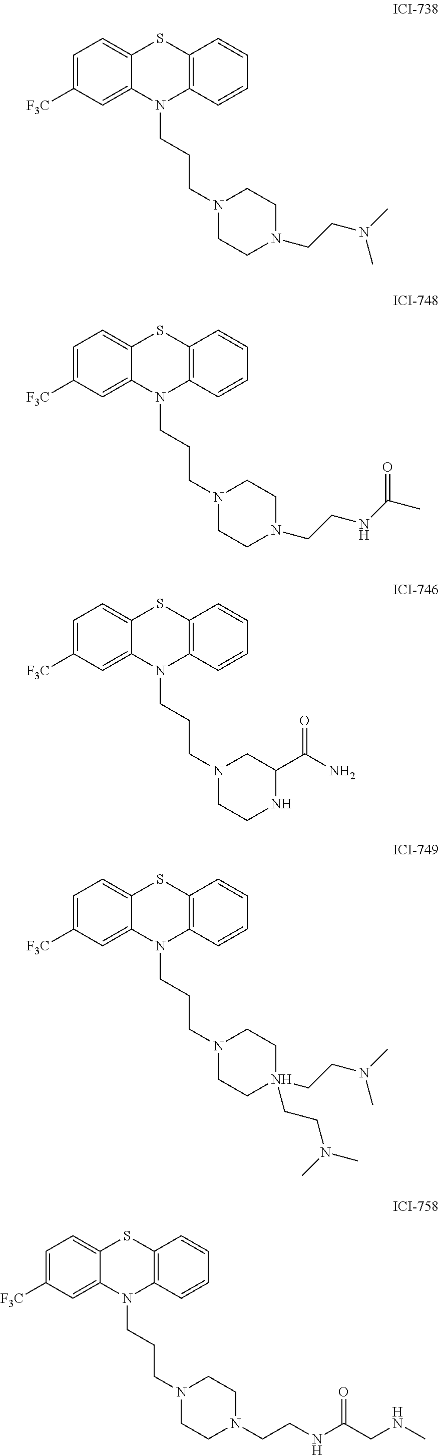

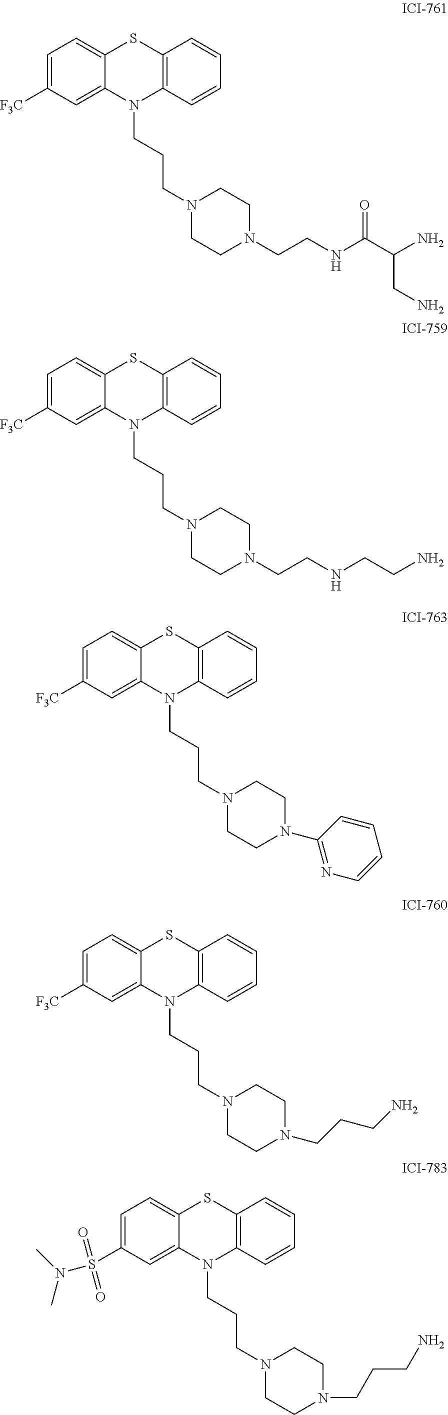

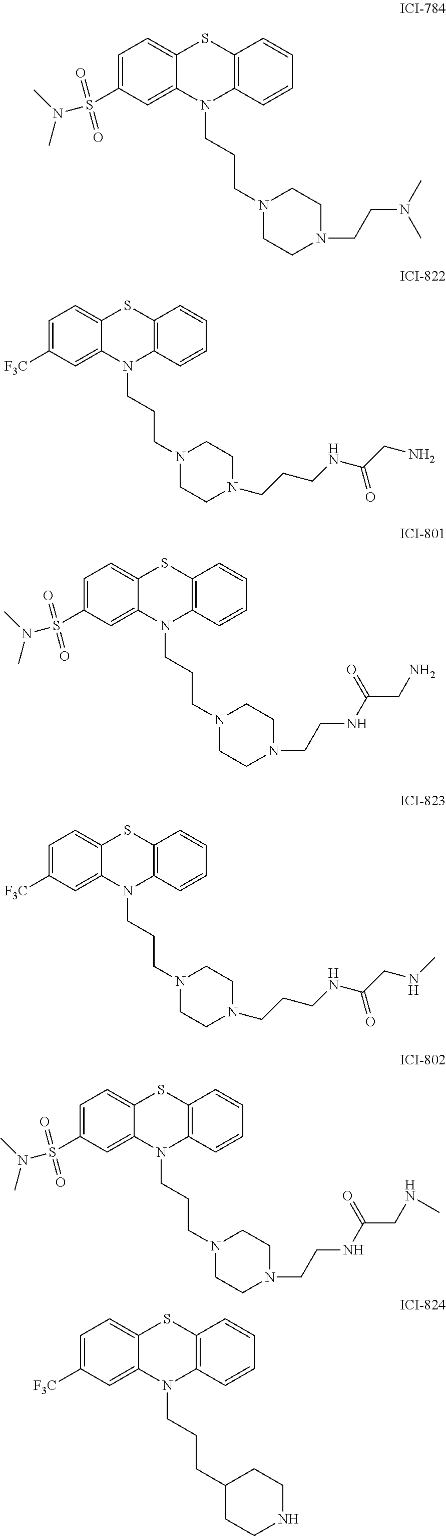

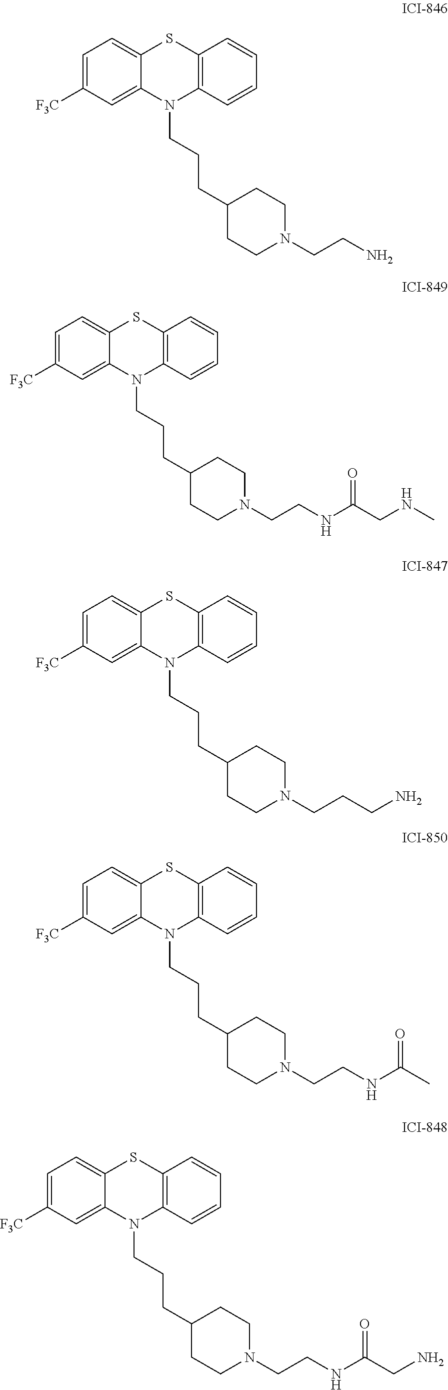

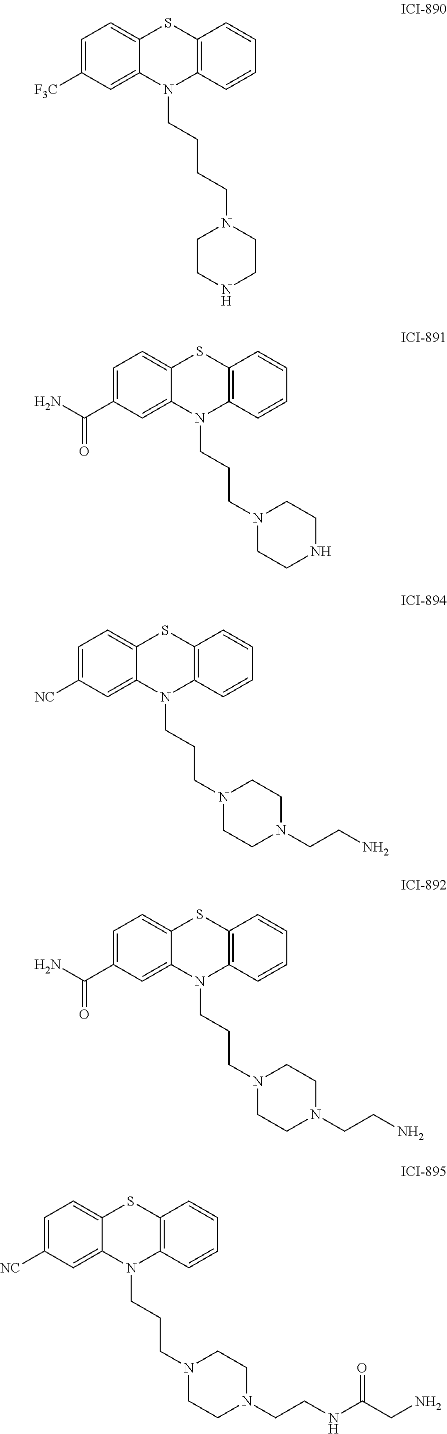

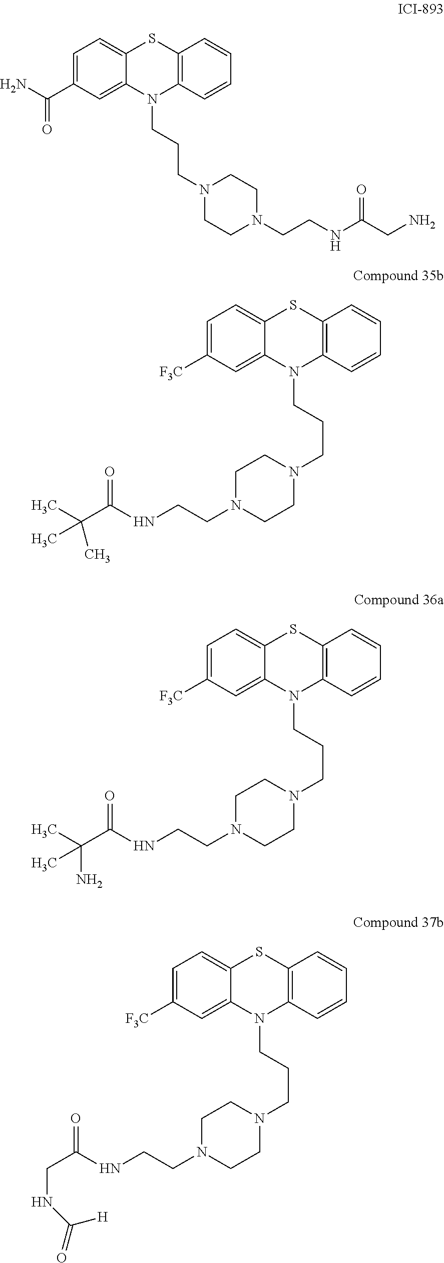

The compositions of the invention may further comprise a pharmaceutically acceptable carrier.

The compounds of the present invention can be used or administered as a pharmaceutically acceptable salt. The phrase “pharmaceutically acceptable salt(s)”, as used herein, unless otherwise indicated, includes salts of acidic or basic groups which may be present in the compounds disclosed herein. The compounds disclosed herein that are basic in nature are capable of forming a wide variety of salts with various inorganic and organic acids. The acids that may be used to prepare pharmaceutically acceptable acid addition salts of such basic compounds of the present 5-HT receptor antagonists are those that form non-toxic acid addition salts, i.e., salts containing pharmacologically acceptable anions, such as the acetate, benzenesulfonate, benzoate, bicarbonate, bisulfate, bitartrate, borate, bromide, calcium edetate, camsylate, carbonate, chloride, clavulanate, citrate, dihydrochloride, edetate, dislyate, estolate, esylate, ethylsuccinate, fumarate, gluceptate, gluconate, glutamate, glycollylarsanilate, hexylresorcinate, hydrabamine, hydrobromide, hydrochloride, iodide, isothionate, lactate, lactobionate, laurate, malate, maleate, mandelate, mesylate, methylsulfate, mutate, napsylate, nitrate, oleate, oxalate, pamoate (embonate), palmitate, pantothenate, phospate/diphosphate, polygalacturonate, salicylate, stearate, subacetate, succinate, tannate, tartrate, teoclate, tosylate, triethiodode, and valerate salts. Since a single compound of the present invention may include more than one acidic or basic moieties, the compounds of the present invention may include mono, di or tri-salts in a single compound.

The 5-HT receptor antagonists of the present invention that are acidic in nature are capable of forming base salts with various pharmacologically acceptable cations. Examples of such salts include the alkali metal or alkaline earth metal salts and, particularly, the calcium, magnesium, sodium and potassium salts of the compounds of the present invention.

This invention also encompasses pharmaceutical compositions comprising prodrugs of the present 5-HT receptor antagonists. Compounds of formula I, II or III and the other 5-HT receptor antagonists disclosed herein having free amino, amido, hydroxy or carboxylic groups can be converted into prodrugs. Prodrugs include compounds wherein an amino acid residue, or a polypeptide chain of two or more (e.g., two, three or four) amino acid residues is covalently joined through an amide or ester bond to a free amino, hydroxy or carboxylic acid group of compounds disclosed herein. The amino acid residues include but are not limited to the 20 naturally occurring amino acids commonly designated by three letter symbols and also includes 4-hydroxyproline, hydroxylysine, demosine, isodemosine, 3-methylhistidine, norvalin, beta-alanine, gamma-aminobutyric acid, citrulline homocysteine, homoserine, ornithine and methionine sulfone. Additional types of prodrugs are also encompassed. For instance, free carboxyl groups can be derivatized as amides or alkyl esters. Free hydroxy groups may be derivatized using groups including but not limited to hemisuecinates, phosphate esters, dimethylaminoacetates, and phosphoryloxymethyloxycarbonyls, as outlined in Advanced Drug Delivery Reviews, 1996, 19: 115. Carbamate prodrugs of hydroxy and amino groups are also included, as are carbonate prodrugs, sulfonate esters and sulfate esters of hydroxy groups. Derivatization of hydroxy groups as (acyloxy)methyl and (acyloxy)ethyl ethers wherein the acyl group may be an alkyl ester, optionally substituted with groups including but not limited to ether, amine and carboxylic acid functionalities, or where the acyl group is an amino acid ester as described above, are also encompassed. Free amines can also be derivatized as amides, sulfonamides or phosphonamides. All of these prodrug moieties may incorporate groups including but not limited to ether, amine and carboxylic acid functionalities.

The present invention also includes isotopically-labeled compounds, which are identical to those recited in the 5-HT receptor antagonists of the invention, but for the fact that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorous, fluorine and chlorine, such as ²H, ³H, ¹³C, ¹⁴C, ¹⁵N, ¹⁸O, ¹⁷O, ³¹P, ³²P, ³⁵S, ¹⁸F, and ³⁶Cl, respectively. Compounds of the present invention, prodrugs thereof; and pharmaceutically acceptable salts of said compounds or of said prodrugs which contain the aforementioned isotopes and/or other isotopes of other atoms are within the scope of this invention. Certain isotopically-labeled compounds of the present invention, for example those into which radioactive isotopes such as ³H and ¹⁴C are incorporated, are useful in drug and/or substrate tissue distribution assays. Tritiated, i.e., ³H, and ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labeled compounds of formula I, II or III of this invention and prodrugs thereof can generally be prepared by carrying out the procedures disclosed herein and known in the art by substituting a readily available isotopically labeled reagent for a non-isotopically labeled reagent.

The compounds of the present invention can also be combined with other compounds useful in the treatment of diseases such as autoimmune diseases, lymphomas, myelomas, and transplant rejection. Such compounds include, but are not limited to, the following therapeutic agents: dexamethasone, melphalan, doxorubicin, bortezomib, lenalidomide, thalidomide, and other agents, such as, but not limited to, regulators of gene expression (e.g., steroids and glucocorticoids, alkylating agents that are known mutagens (e.g., cyclophosphamide), inhibitors of kinases and phosphatases which act on the calcineurin and JNK/p38 kinase pathways and the cyclin kinase cascade (e.g., CyclosporinA, Tacrolimus [FK506], and Rapamycin), inhibitors of de novo purine synthesis which act as inhibitors of guanosine nucleotide synthesis and are used to prevent allograft rejection and to treat ongoing rejection (e.g., Mycophenolate motefil), and inhibitors of de novo pyrimidine synthesis which are used to treat patients afflicted with rheumatoid arthritis (e.g., Leflunomide), TNF-α inhibitors, such as Adalimumab, Etanercept, Infliximab, and other immunomodulating agents, such as methotrexate, azathioprine, natalizumab, and mercaptopurine. Therefore, the invention encompasses a composition comprising a 5-HT receptor antagonist disclosed herein, such as a 5-HT receptor antagonist of formula I, II or III, and immunomodulating agent disclosed elsewhere herein.

A composition comprising a compound of the present invention, such as the 5-HT receptor antagonist of formula I, II or III or another compound disclosed herein, and a therapeutic agent are within the scope of the present invention, whether physically combined prior to administration to a patient or combined within a patient.

### Methods

The present method includes compositions and methods useful in preventing or treating PAH in a mammal. In a non-limiting aspect, as demonstrated by the data disclosed herein, administration of the compositions of the invention to a mammal with PAH decreases the mammal's PAP, RVSP, and RV/BW parameters, without evidence of physical or behavioral drug-related toxicity to the mammal.

The present invention further includes a method of inducing apoptosis in a lymphocyte. The method comprises inhibiting the interaction of serotonin with a serotonin receptor by contacting a lymphocyte with a 5-HT receptor antagonist, such as the 5-HT receptor antagonist of formula I, II or III or a 5-HT receptor antagonist disclosed elsewhere herein. In a preferred embodiment, the 5-HT receptor antagonist is a 5-HT receptor antagonist of formula I, II or III. More preferably, the 5-HT receptor antagonist is selected from, among others, ICI-685, ICI-715, ICI-735, ICI-824, ICI-846, ICI-847, ICI-848, ICI-849, ICI-890, ICI-894, ICI-953, ICI-954, Compound 36a, Compound 37b or Compound 35b. This is because, as demonstrated by the data disclosed herein, contacting a lymphocyte with a 5-HT receptor antagonist of the present invention results in, among other things, an inhibition of proliferation of a variety of lymphocytes, including T-cells and B-cells, In addition, the data disclosed herein demonstrates that contacting a lymphocyte with a 5-HT receptor antagonist of the present application results in apoptosis of the lymphocyte in a dose and time dependent manor. Thus, the present invention comprises inducing apoptosis in a lymphocyte and a method of inhibiting proliferation of a lymphocyte by contacting the lymphocyte with a 5-HT receptor antagonist.

The present invention also comprises a method of treating a mammal, preferably a human, having a disease characterized by abnormal lymphocyte proliferation where inhibiting lymphocyte proliferation or inducing apoptosis in the abnormally proliferating lymphocytes results in treatment of the disease. The method comprises administering an effective amount of a 5-HT receptor antagonist to a mammal, preferably a human, in need thereof. As demonstrated by the data disclosed herein, administration of a 5-HT receptor antagonist of the present invention results in, among other things, a rapid cessation of proliferation of various types of lymphocytes, including, but not limited to, T-cells and B-cells. In addition, according to the data presented herein, administration of a 5-HT receptor antagonist of the present invention results in apoptosis in the lymphocyte. Inducing apoptosis or inhibiting proliferation of a lymphocyte prevents or treats the generation of an immune response, such as those common to autoimmune diseases and transplant rejection, and also treats lymphatic neoplasias, including lymphomas and myelomas.

One of skill in the art would also appreciate, based upon the disclosure provided herein, that the invention encompasses using a 5-HT receptor antagonist that is water soluble and that does not substantially cross the blood-brain barrier. This is because one skilled in the art would understand that because serotonin receptors are found on neural cells and, as now disclosed, on cells of the immune system, including tumors derived from such cells (e.g., multiple myelomas, and the like), it is desirable, but not necessary, to inhibit signaling via serotonin receptor on an immune cell while not affecting serotonin signaling via a serotonin receptor on a neural cell. In such instances, administering a compound that inhibits signaling but does not cross the blood-brain barrier where it would affect serotonin signaling in neural cells is desirable.

Accordingly, the present invention encompasses using a compound that, while inhibiting serotonin signaling via a serotonin receptor on a cell, does not substantially cross the blood-brain barrier. Such compounds are disclosed elsewhere herein and include the 5-HT receptor antagonist of formula I, II or III, as well as those disclosed elsewhere herein, but preferably includes ICI-685, ICI-715, ICI-735, ICI-824, ICI-846, ICI-847, ICI-848, ICI-849, ICI-890, ICI-894, ICI-953, ICI-954, Compound 36a, Compound 37b or Compound 35b.

One skilled in the art would understand, based upon the disclosure provided herein, that methods to modify a compound to affect its ability to cross the blood-brain barrier are well-known in the art, which also teaches a wide plethora of assays for assessing the ability of a substance to cross the barrier. One such method is disclosed herein, i.e., adding various sidegroups to a compound such as fluphenazine, thereby decreasing the ability of the modified fluphenazine to cross the blood-brain barrier. The modified fluphenazine compounds, designated, e.g., formula I, II or III, are disclosed herein, but the present application is in no way limited to these or any other particular derivatives of fluphenazine. Instead, the invention encompasses any compound having the desired immunomodulatory characteristics of the inhibitors of the invention, while also possessing the desired reduced ability to cross the blood-brain barrier. The production and identification of compounds having these characteristics are routine in the art, as are assays for assessing the permeability of a compound through the blood-brain barrier. Such assays are exemplified herein, as are methods of producing compounds of interest having the desired characteristics. Nonetheless, the present invention is in no way limited to these, or any other, methods in particular; rather, it includes methods of producing and identifying compounds that do not substantially cross the blood-brain barrier and still inhibit serotonin signaling via a serotonin receptor such as those disclosed herein, known in the art, or to be developed in the future.

The present invention can be used to treat a variety of autoimmune diseases, including, but not limited to, myasthenia gravis, idiopathic inflammatory myopathy, chronic neutropenia, rheumatoid arthritis, idiopathic thromcytopenia purpura, autoimmune hemolytic syndromes, antiphospholipid antibody syndromes, inflammatory bowel disease, Crohn's disease, ulcerative colitis, myocarditis, Guillian-Barre Syndrome, vasculitis, multiple sclerosis, neuromyelitis optica (devic's syndrome), lymphocytic hypophysitis, Graves disease, Addison's disease, hypoparathroidism, type 1 diabetes, systemic lupus erythematosus, pemphigus vulgaris, bullous pemphigoid, psoriasis, psoriatic arthritis, endometriosis, autoimmune orchitis, dystrophic epidermolysis, sarcoidosis, Wegener's granulomatosis, autoimmune deafness, Sjögren's disease, autoimmune uveoretinitis, interstitial cystitis, Goodpasture's syndrome, and fibromyalgia. This is because, as demonstrated by the data disclosed herein, the 5-HT receptor antagonists of the present invention inhibit the proliferation of both T cells and B cells, and additionally induce apoptosis in such lymphocytes, Thus, the methods of the present invention comprise administering an effective amount of a 5-HT receptor antagonist to a mammal, preferably a human, having an autoimmune disease, e.g. psoriasis.

The invention further comprises compounds and methods for treating asthma.

The present invention also comprises compositions and methods for the treatment of immune-cell related diseases and disorders. In an aspect, the disease or disorder is not autoimmune-related.

The present invention further comprises a method of treating organ transplant rejection in a mammal in need thereof. Specifically contemplated in the present invention are methods of treating graft versus host disease (GVHD) and organ transplant rejection by administering a 5-HT receptor antagonist disclosed herein to a patient suffering from GVHD and/or organ transplant rejection. The present invention comprises methods of treating, for example, transplant rejection of thoracic organs, such as heart transplants, lung transplants and en bloc heart/lung transplants. The methods of the invention further comprise treating rejection of abdominal organs, such as liver, kidney, pancreas, small bowel and combined transplants, such as kidney/pancreas transplants, liver/kidney transplants, and combined liver/small bowel transplants. The methods of the present invention further comprise treatment after rejection of a hand, cornea, skin or face transplant. In addition, the methods of the present invention can be used to treat rejection of tissues, cells and fluids that are commonly transplanted, including, but not limited to, pancreatic islet cells (islets of Langerhans), bone marrow transplants, adult stem cell transplants, blood transfusions, blood vessel grafts, heart valve grafts, where autologous, allogenic or xenogenic, and bone grafts. This is because, as demonstrated by the data disclosed herein, administering the 5-HT receptor antagonists of the present invention results in inhibited proliferation of T cells, one of the effector cells in transplant and graft rejection, and induces apoptosis in B cells, which produce anti-graft antibodies. Thus, the invention encompasses a method of treating transplant rejection by administering an effective amount of the 5-HT receptor antagonists of the present invention to a mammal, preferably a human, in need thereof.

The methods of the present invention further comprise treating a mammal having an autoimmune disease or a mammal rejecting an organ or tissue transplant with a combination of a 5-HT receptor antagonist with another immunomodulatory agent. Such immunomodulatory agents include, but are not limited to, other agents, such as, but not limited to, regulators of gene expression (e.g., steroids and glucocorticoids, alkylating agents that are known mutagens (e.g., cyclophosphamide), inhibitors of kinases and phosphatases which act on the calcineurin and JNK/p38 kinase pathways and the cyclin kinase cascade (e.g., CyclosporinA, Tacrolimus [FK506], and Rapamycin), inhibitors of de novo purine synthesis which act as inhibitors of guanosine nucleotide synthesis and are used to prevent allograft rejection and to treat ongoing rejection (e.g., Mycophenolate motefil), and inhibitors of de novo pyrimidine synthesis which are used to treat patients afflicted with rheumatoid arthritis (e.g., Leflunomide), TNF-α inhibitors, such as Adalimumab, Etanercept, Infliximab, and other immunomodulating agents, such as methotrexate, azathioprine, natalizumab, and mercaptopurine.

The immunomodulatory agents of the present invention can be combined with a 5-HT receptor antagonist of the present invention, such as the 5-HT receptor antagonist of formula I, II or III, ICI-685, ICI-715, ICI-735, ICI-824, ICI-846, ICI-847, ICI-848, ICI-849, ICI-890, ICI-894, ICI-953, ICI-954, Compound 36a, Compound 37b or Compound 35b, to treat a patient having an autoimmune disease or a patient experiencing transplant rejection. The immunomodulatory agent can be combined with a 5-HT receptor antagonist and delivered as one dose or a series of doses, either together or separately. Methods for the combinations of drugs and dosages are described elsewhere herein.

The present invention further comprises a method of treating neoplasias in a human, preferably lymphomas and myelomas. This is because, as demonstrated by the data disclosed herein, neoplastic lymphoma and myeloma cells, when contacted with a 5-HT receptor antagonist of the present invention, cease proliferating and apoptose. Thus, the present invention comprises methods for treating a mammal, preferably a human, having a lymphoma or a myeloma, the method comprising administering to the mammal an effective amount of a 5-HT receptor antagonist of the present invention. Such 5-HT receptor antagonists include, but are not limited to the 5-HT receptor antagonist of formula I, II or III, ICI-685, ICI-715, ICI-735, ICI-824, ICI-846, ICI-847, ICI-848, ICI-849, ICI-890, ICI-894, ICI-953, and ICI-954.

A mammal having a lymphoma can be treated using the methods of the present invention by administering to the mammal an effective amount of a 5-HT receptor antagonist of the present invention. Lymphomas that can be treated using the methods of the present invention include, but are not limited to, non-Hodgkin lymphomas, such as T cell prolymphocytic leukemia, T cell large granular lymphocytic leukemia, aggressive NK cell leukemia, adult T cell leukemia/lymphoma, extranodal NK/T cell lymphoma, nasal type, enteropathy-type T cell lymphoma, hepatosplenic T cell lymphoma, blastic NK cell lymphoma, mycosis fungoides/Sezary syndrome, primary cutaneous CD30-positive T cell lymphoproliferative disorders, primary cutaneous anaplastic large cell lymphoma, lymphomatoid papulosis, angioimmunoblastic T cell lymphoma, peripheral T cell lymphoma, unspecified, and anaplastic large cell lymphoma. The present invention further comprises methods of treating Hodgkin's lymphomas by administering to a patient having a Hodgkin's lymphoma an effective amount of a 5-HT receptor antagonist of the present invention. Such Hodgkin's lymphomas include, but are not limited to, nodular lymphocyte-predominant Hodgkin lymphoma and classical Hodgkin lymphoma, including nodular sclerosis, mixed cellularity Hodgkin's lymphoma, lymphocyte-rich Hodgkin's lymphoma and lymphocyte depleted Hodgkin's lymphoma.

The methods of the present invention further comprise treating a mammal, preferably a human, with myeloma. This is because, as demonstrated by the data disclosed herein, the 5-HT receptor antagonists of the present invention inhibit the proliferation and induce apoptosis in a variety of common myeloma cells, including primary multiple myeloma cells from treated and untreated patients, and multiple myeloma cells resistant to conventional multiple myeloma therapeutics, such as dexamethasone and melphalan.

The methods of the present invention are used to treat multiple myeloma in a patient in need thereof. The method comprises administering to a patient in need thereof a fluphenazine inhibitor of the present invention. This is because, as disclosed elsewhere herein, contacting a multiple myeloma cell with a 5-HT receptor antagonist of the present invention, such as the 5-HT receptor antagonist of formula I, II or III, ICI-685, ICI-715, ICI-735, ICI-824, ICI-846, ICI-847, ICI-848, ICI-849, ICI-890, ICI-894, ICI-953, ICI-954, Compound 36a, Compound 37b or Compound 35b causes an inhibition of proliferation of the multiple myeloma cell as well as induces apoptosis in a multiple myeloma cell. Thus, the present invention comprises a method of treating multiple myeloma in a mammal, preferably a human. Further, as demonstrated by the data herein, the present invention comprises a method of inducing apoptosis in a multiple myeloma cell, whether in a patient or isolated from the patient, by contacting the multiple myeloma cell with a fluphenazine inhibitor of the present invention.

The present invention is used to treat multiple myeloma of all stages on the International Staging System (ISS), including Stage I: β2-microglobulin<3.5 mg/L, albumin≧3.5 g/dL; Stage II: β2-microglobulin<3.5 mg/L and albumin<3.5 g/dL or β2-microglobulin between 3.5 and 5.5 mg/L; and Stage III: β2-microglobulin>5.5 mg/L. In addition, the methods of the present invention comprise combination therapy for treating multiple myeloma. The combinations of the present invention comprise a 5-HT receptor antagonist, such as the 5-HT receptor antagonist of formula I, II or III, ICI-685, ICI-715, ICI-735, ICI-824, ICI-846, ICI-847, ICI-848, ICI-849, ICI-890, ICI-894, ICI-953, ICI-954, Compound 36a, Compound 37b or Compound 35b combined with additional agents and therapies used for treating multiple myeloma. Specifically contemplated combination therapies include a 5-HT receptor antagonist administered before or after allogeneic or autologous stem cell transplantation, a 5-HT receptor antagonist and a bisphosphonate (e.g. pamidronate) to prevent fractures, and a 5-HT receptor antagonist and erythropoietin to treat anemia associated with multiple myeloma.

Additional combination therapies specifically contemplated in the present invention include a 5-HT receptor antagonist and dexamethasone with or without thalidomide, a 5-HT receptor antagonist and thalidomide, a 5-HT receptor antagonist and vincristine, a 5-HT receptor antagonist and doxorubicin, a 5-HT receptor antagonist and melphalan, and a 5-HT receptor antagonist with melphalan and prednisone. In relapsed patients, or patients otherwise not responding to conventional multiple myeloma therapies, the invention encompasses methods of treating multiple myeloma in a patient comprising administering combinations of a 5-HT receptor antagonist and cyclophosphamide, a 5-HT receptor antagonist and bortezomib or a 5-HT receptor antagonist and lenalidomide. The renal failure that often accompanies multiple myeloma can be treated using a 5-HT receptor antagonist of the present invention and kidney dialysis.

The combinations of a 5-HT receptor antagonist and another multiple myeloma therapy are, as demonstrated by the data disclosed herein, effective at inhibiting proliferation and inducing apoptosis in multiple myeloma cells. As a non-limiting example, nanomolar concentrations of the present 5-HT receptor antagonists and other multiple myeloma therapies resulted in, among other things, increased apoptosis and decreased proliferation when compared to conventional multiple myeloma therapies alone.

As further demonstrated by the data disclosed herein, the 5-HT receptor antagonists of the present invention induce apoptosis and inhibit proliferation in a variety of lymphocytes, and thus are useful in the treatment of various immune system related diseases. Thus, the present invention further comprises a method of inhibiting an immune response in a mammal, preferably a human, by inhibiting serotonin binding with a serotonin receptor by administering a 5-HT receptor antagonist of the present invention, thereby inhibiting an immune reaction by the cell, which in turn inhibits an immune response mediated by that cell. The invention further comprises a method of inhibiting an immune reaction by an immune cell. This is because, as set forth elsewhere herein, inhibition of serotonin binding with a serotonin receptor on the immune cell inhibits activation of the cell, which in turn inhibits an immune reaction by that cell when compared to the immune reaction by that cell in the absence of inhibition of serotonin binding and/or when compared with the immune reaction of an otherwise identical cell wherein serotonin binding with its receptor is not inhibited. The present invention further encompasses a method of inhibiting activation of an immune cell, such as a lymphocyte, in a mammal, preferably, a human, wherein the activation is mediated by activation of a serotonin receptor on the cell. Again, this is because, as more fully set forth elsewhere herein, the data disclosed herein demonstrate that inhibiting serotonin signaling via a serotonin receptor on an immune cell by contacting the cell with a 5-HT receptor antagonist inhibits activation of the cell, and therefore, also inhibits the immune response that would otherwise be produced by that cell.

### Formulation and Administration

The 5-HT receptor antagonist, alone or in combinations described herein, that inhibits the serotonin receptor-mediated signals can be administered to a cell, a tissue, or an animal to inhibit interaction of serotonin with a serotonin type receptor on a cell, a tissue, or in an animal. Methods for the safe and effective administration of the 5-HT receptor antagonists described herein are know to those skilled in the art. For instance, the administration of serotonin antagonists is described in the standard literature. That is, the administration of many serotonin-affecting agents, serotonin receptor antagonists, and fluphenazine is set forth in the Physician's Desk Reference (1996 edition, Medical Economics Co., Montvale, N.J.), the disclosure of which is incorporated by reference as if set forth in its entirety herein.

For administration of a 5-HT receptor antagonist of the present invention to a mammal, the compound can be suspended in any pharmaceutically acceptable carrier, for example, sterile water or a buffered aqueous carriers, such as glycerol, water, saline, ethanol and other pharmaceutically acceptable salt solutions such as phosphates and salts of organic acids. Examples of these and other pharmaceutically acceptable carriers are described in Remington's Pharmaceutical Sciences (1991, Mack Publication Co., New Jersey), the disclosure of which is incorporated by reference as if set forth in its entirety herein.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides.

Pharmaceutical compositions that are useful in the methods of the invention may be administered, prepared, packaged, and/or sold in formulations suitable for oral, rectal, vaginal, parenteral, topical, pulmonary, intranasal, buccal, ophthalmic, bolus injection, or another route of administration. Other contemplated formulations include projected nanoparticles, liposomal preparations, resealed erythrocytes containing the active ingredient, and immunologically-based formulations.

The compositions of the invention may be administered via numerous routes, including, but not limited to, oral, rectal, vaginal, parenteral, topical, pulmonary, intranasal, buccal, or ophthalmic administration routes. The route(s) of administration will be readily apparent to the skilled artisan and will depend upon any number of factors including the type and severity of the disease being treated, the type and age of the veterinary or human patient being treated, and the like,

Pharmaceutical compositions that are useful in the methods of the invention may be administered systemically in oral solid formulations, ophthalmic, suppository, aerosol, topical or other similar formulations. In addition to the compound such as heparan sulfate, or a biological equivalent thereof, such pharmaceutical compositions may contain pharmaceutically-acceptable carriers and other ingredients known to enhance and facilitate drug administration.

Compounds which are identified using any of the methods described herein may be formulated and administered to a mammal for treatment of immune system conditions (i.e., autoimmune diseases and allograft rejection), are now described.

The invention encompasses the preparation and use of pharmaceutical compositions comprising a compound useful for treatment of a wide variety of disorders such as T cell lymphomas, autoimmune disorders (see infra), complications arising from solid organ transplants, skin graft rejection, graft versus host disease in bone marrow transplants, multiple myeloma, and the like.

The pharmaceutical compositions described herein can be prepared alone, in a form suitable for administration to a subject, or the pharmaceutical composition may comprise the active ingredient and one or more pharmaceutically acceptable carriers, one or more additional ingredients, or some combination of these. The active ingredient may be present in the pharmaceutical composition in the form of a physiologically acceptable ester or salt, such as in combination with a physiologically acceptable cation or anion, as is well known in the art.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

Although the descriptions of pharmaceutical compositions provided herein are principally directed to pharmaceutical compositions that are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the invention is contemplated include, but are not limited to, humans and other primates, mammals including commercially relevant mammals such as cattle, pigs, horses, sheep, cats, and dogs.

A pharmaceutical composition of the invention may be prepared, packaged, or sold in bulk, as a single unit dose, or as a plurality of single unit doses. As used herein, a “unit dose” is a discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

In addition to the active ingredient, a pharmaceutical composition of the invention may further comprise one or more additional pharmaceutically active agents. Particularly contemplated additional agents include anti-emetics and scavengers such as cyanide and cyanate scavengers.

Controlled- or sustained-release formulations of a pharmaceutical composition of the invention may be made using conventional technology.

A formulation of a pharmaceutical composition of the invention suitable for oral administration may be prepared, packaged, or sold in the form of a discrete solid dose unit including, but not limited to, a tablet, a hard or soft capsule, a cachet, a troche, or a lozenge, each containing a predetermined amount of the active ingredient. Other formulations suitable for oral administration include, but are not limited to, a powdered or granular formulation, an aqueous or oily suspension, an aqueous or oily solution, or an emulsion.

As used herein, an “oily” liquid is one which comprises a carbon-containing liquid molecule and which exhibits a less polar character than water.

A tablet comprising the active ingredient may, for example, be made by compressing or molding the active ingredient, optionally with one or more additional ingredients. Compressed tablets may be prepared by compressing, in a suitable device, the active ingredient in a free-flowing form such as a powder or granular preparation, optionally mixed with one or more of a binder, a lubricant, an excipient, a surface active agent, and a dispersing agent. Molded tablets may be made by molding, in a suitable device, a mixture of the active ingredient, a pharmaceutically acceptable carrier, and at least sufficient liquid to moisten the mixture. Pharmaceutically acceptable excipients used in the manufacture of tablets include, but are not limited to, inert diluents, granulating and disintegrating agents, binding agents, and lubricating agents. Known dispersing agents include, but are not limited to, potato starch and sodium starch glycollate. Known surface active agents include, but are not limited to, sodium lauryl sulphate. Known diluents include, but are not limited to, calcium carbonate, sodium carbonate, lactose, microcrystalline cellulose, calcium phosphate, calcium hydrogen phosphate, and sodium phosphate. Known granulating and disintegrating agents include, but are not limited to, corn starch and alginic acid. Known binding agents include, but are not limited to, gelatin, acacia, pre-gelatinized maize starch, polyvinylpyrrolidone, and hydroxypropyl methylcellulose. Known lubricating agents include, but are not limited to, magnesium stearate, stearic acid, silica, and talc.

Tablets may be non-coated or they may be coated using known methods to achieve delayed disintegration in the gastrointestinal tract of a subject, thereby providing sustained release and absorption of the active ingredient. By way of example, a material such as glyceryl monostearate or glyceryl distearate may be used to coat tablets. Further by way of example, tablets may be coated using methods described in U.S. Pat. Nos. 4,256,108; 4,160,452; and 4,265,874 to form osmotically-controlled release tablets. Tablets may further comprise a sweetening agent, a flavoring agent, a coloring agent, a preservative, or some combination of these in order to provide pharmaceutically elegant and palatable preparation.

Hard capsules comprising the active ingredient may be made using a physiologically degradable composition, such as gelatin. Such hard capsules comprise the active ingredient, and may further comprise additional ingredients including, for example, an inert solid diluent such as calcium carbonate, calcium phosphate, or kaolin.

Soft gelatin capsules comprising the active ingredient may be made using a physiologically degradable composition, such as gelatin. Such soft capsules comprise the active ingredient, which may be mixed with water or an oil medium such as peanut oil, liquid paraffin, or olive oil.

Liquid formulations of a pharmaceutical composition of the invention which are suitable for oral administration may be prepared, packaged, and sold either in liquid form or in the form of a dry product intended for reconstitution with water or another suitable vehicle prior to use.

Liquid suspensions may be prepared using conventional methods to achieve suspension of the active ingredient in an aqueous or oily vehicle. Aqueous vehicles include, for example, water and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin. Liquid suspensions may further comprise one or more additional ingredients including, but not limited to, suspending agents, dispersing or wetting agents, emulsifying agents, demulcents, preservatives, buffers, salts, flavorings, coloring agents, and sweetening agents. Oily suspensions may further comprise a thickening agent. Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, hydroxypropylmethylcellulose. Known dispersing or wetting agents include, but are not limited to, naturally-occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g., polyoxyethylene stearate, heptadecaethyleneoxycetanol, polyoxyethylene sorbitol monooleate, and polyoxyethylene sorbitan monooleate, respectively). Known emulsifying agents include, but are not limited to, lecithin and acacia. Known preservatives include, but are not limited to, methyl, ethyl, or n-propyl-para-hydroxybenzoates, ascorbic acid, and sorbic acid. Known sweetening agents include, for example, glycerol, propylene glycol, sorbitol, sucrose, and saccharin. Known thickening agents for oily suspensions include, for example, beeswax, hard paraffin, and cetyl alcohol.

Liquid solutions of the active ingredient in aqueous or oily solvents may be prepared in substantially the same manner as liquid suspensions, the primary difference being that the active ingredient is dissolved, rather than suspended in the solvent. Liquid solutions of the pharmaceutical composition of the invention may comprise each of the components described with regard to liquid suspensions, it being understood that suspending agents will not necessarily aid dissolution of the active ingredient in the solvent. Aqueous solvents include, for example, water and isotonic saline. Oily solvents include, for example, almond oil, oily esters, ethyl alcohol, vegetable oils such as arachis, olive, sesame, or coconut oil, fractionated vegetable oils, and mineral oils such as liquid paraffin.

Powdered and granular formulations of a pharmaceutical preparation of the invention may be prepared using known methods. Such formulations may be administered directly to a subject, used, for example, to form tablets, to fill capsules, or to prepare an aqueous or oily suspension or solution by addition of an aqueous or oily vehicle thereto. Each of these formulations may further comprise one or more of dispersing or wetting agent, a suspending agent, and a preservative. Additional excipients, such as fillers and sweetening, flavoring, or coloring agents, may also be included in these formulations.

A pharmaceutical composition of the invention may also be prepared, packaged, or sold in the form of oil-in-water emulsion or a water-in-oil emulsion. The oily phase may be a vegetable oil such as olive or arachis oil, a mineral oil such as liquid paraffin, or a combination of these. Such compositions may further comprise one or more emulsifying agents such as naturally occurring gums such as gum acacia or gum tragacanth, naturally-occurring phosphatides such as soybean or lecithin phosphatide, esters or partial esters derived from combinations of fatty acids and hexitol anhydrides such as sorbitan monooleate, and condensation products of such partial esters with ethylene oxide such as polyoxyethylene sorbitan monooleate. These emulsions may also contain additional ingredients including, for example, sweetening or flavoring agents.

A pharmaceutical composition of the invention may be prepared, packaged, or sold in a formulation suitable for rectal administration. Such a composition may be in the form of, for example, a suppository, a retention enema preparation, and a solution for rectal or colonic irrigation.

Suppository formulations may be made by combining the active ingredient with a non-irritating pharmaceutically acceptable excipient which is solid at ordinary room temperature (i.e., about 20° C.) and which is liquid at the rectal temperature of the subject (i.e., about 37° C. in a healthy human). Suitable pharmaceutically acceptable excipients include, but are not limited to, cocoa butter, polyethylene glycols, and various glycerides. Suppository formulations may further comprise various additional ingredients including, but not limited to, antioxidants and preservatives.

Retention enema preparations or solutions for rectal or colonic irrigation may be made by combining the active ingredient with a pharmaceutically acceptable liquid carrier. As is well known in the art, enema preparations may be administered using, and may be packaged within, a delivery device adapted to the rectal anatomy of the subject. Enema preparations may further comprise various additional ingredients including, but not limited to, antioxidants and preservatives.

A pharmaceutical composition of the invention may be prepared, packaged, or sold in a formulation suitable for vaginal administration. Such a composition may be in the form of for example, a suppository, an impregnated or coated vaginally-insertable material such as a tampon, a douche preparation, or gel or cream or a solution for vaginal irrigation.

Methods for impregnating or coating a material with a chemical composition are known in the art, and include, but are not limited to methods of depositing or binding a chemical composition onto a surface, methods of incorporating a chemical composition into the structure of a material during the synthesis of the material (i.e., such as with a physiologically degradable material), and methods of absorbing an aqueous or oily solution or suspension into an absorbent material, with or without subsequent drying.

Douche preparations or solutions for vaginal irrigation may be made by combining the active ingredient with a pharmaceutically acceptable liquid carrier. As is well known in the art, douche preparations may be administered using, and may be packaged within, a delivery device adapted to the vaginal anatomy of the subject. Douche preparations may further comprise various additional ingredients including, but not limited to, antioxidants, antibiotics, antifungal agents, and preservatives.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, intravenous, subcutaneous, intraperitoneal, intramuscular, intrasternal injection, bolus injections, and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e., powder or granular) form for reconstitution with a suitable vehicle (e.g., sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally-administrable formulations which are useful include those which comprise the active ingredient in microcrystalline form, in a liposomal preparation, or as a component of a biodegradable polymer systems. Compositions for sustained release or implantation may comprise pharmaceutically acceptable polymeric or hydrophobic materials such as an emulsion, an ion exchange resin, a sparingly soluble polymer, or a sparingly soluble salt.

Formulations suitable for topical administration include, but are not limited to, liquid or semi-liquid preparations such as liniments, lotions, oil-in-water or water-in-oil emulsions such as creams, ointments or pastes, and solutions or suspensions. Topically-administrable formulations may, for example, comprise from about 0.1% to about 10% (w/w) active ingredient, although the concentration of the active ingredient may be as high as the solubility limit of the active ingredient in the solvent. Formulations for topical administration may further comprise one or more of the additional ingredients described herein.

A pharmaceutical composition of the invention may be prepared, packaged, or sold in a formulation suitable for pulmonary administration via the buccal cavity. Such a formulation may comprise dry particles which comprise the active ingredient and which have a diameter in the range from about 0.5 to about 7 nanometers, and preferably from about 1 to about 6 nanometers. Such compositions are conveniently in the form of dry powders for administration using a device comprising a dry powder reservoir to which a stream of propellant may be directed to disperse the powder or using a self-propelling solvent/powder-dispensing container such as a device comprising the active ingredient dissolved or suspended in a low-boiling propellant in a sealed container. Preferably, such powders comprise particles wherein at least 98% of the particles by weight have a diameter greater than 0.5 nanometers and at least 95% of the particles by number have a diameter less than 7 nanometers. More preferably, at least 95% of the particles by weight have a diameter greater than 1 nanometer and at least 90% of the particles by number have a diameter less than 6 nanometers. Dry powder compositions preferably include a solid fine powder diluent such as sugar and are conveniently provided in a unit dose form.

Low boiling propellants generally include liquid propellants having a boiling point of below 65° F. at atmospheric pressure. Generally the propellant may constitute 50 to 99.9% (w/w) of the composition, and the active ingredient may constitute 0.1 to 20% (w/w) of the composition. The propellant may further comprise additional ingredients such as a liquid non-ionic or solid anionic surfactant or a solid diluent (preferably having a particle size of the same order as particles comprising the active ingredient).

Pharmaceutical compositions of the invention formulated for pulmonary delivery may also provide the active ingredient in the form of droplets of a solution or suspension. Such formulations may be prepared, packaged, or sold as aqueous or dilute alcoholic solutions or suspensions, optionally sterile, comprising the active ingredient, and may conveniently be administered using any nebulization or atomization device. Such formulations may further comprise one or more additional ingredients including, but not limited to, a flavoring agent such as saccharin sodium, a volatile oil, a buffering agent, a surface active agent, or a preservative such as methylhydroxybenzoate. The droplets provided by this route of administration preferably have an average diameter in the range from about 0.1 to about 200 nanometers.

The formulations described herein as being useful for pulmonary delivery are also useful for intranasal delivery of a pharmaceutical composition of the invention.

Another formulation suitable for intranasal administration is a coarse powder comprising the active ingredient and having an average particle from about 0.2 to 500 micrometers. Such a formulation is administered in the manner in which snuff is taken, i.e., by rapid inhalation through the nasal passage from a container of the powder held close to the nares.

Formulations suitable for nasal administration may, for example, comprise from about as little as 0.1% (w/w) and as much as 100% (w/w) of the active ingredient, and may further comprise one or more of the additional ingredients described herein.

A pharmaceutical composition of the invention may be prepared, packaged, or sold in a formulation suitable for buccal administration. Such formulations may, for example, be in the form of tablets or lozenges made using conventional methods, and may, for example, 0.1 to 20% (w/w) active ingredient, the balance comprising an orally dissolvable or degradable composition and, optionally, one or more of the additional ingredients described herein. Alternately, formulations suitable for buccal administration may comprise a powder or an aerosolized or atomized solution or suspension comprising the active ingredient. Such powdered, aerosolized, or aerosolized formulations, when dispersed, preferably have an average particle or droplet size in the range from about 0.1 to about 200 nanometers, and may further comprise one or more of the additional ingredients described herein.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials, Other “additional ingredients” which may be included in the pharmaceutical compositions of the invention are known in the art and described, for example in Genaro, ed. (1985, Remington's Pharmaceutical Sciences, Mack Publishing Co., Easton, Pa.), which is incorporated herein by reference.

Typically, dosages of the compound of the invention which may be administered to an animal, preferably a human, will vary depending upon any number of factors, including but not limited to, the type of animal and type of disease state being treated, the age of the animal and the route of administration.

The compound can be administered to an animal as frequently as several times daily, or it may be administered less frequently, such as once a day, once a week, once every two weeks, once a month, or even less frequently, such as once every several months or even once a year or less. The frequency of the dose will be readily apparent to the skilled artisan and will depend upon any number of factors, such as, but not limited to, the type and severity of the disease being treated, the type and age of the animal, and the like. Preferably, the compound is, but need not be, administered as a bolus injection that provides lasting effects for at least one day following injection. The bolus injection can be provided intraperitoneally.

Thus, the skilled artisan would appreciate, once armed with the teachings provided herein, that the invention encompasses administration of a bolus comprising an inhibitor of the interaction of serotonin with a serotonin receptor, preferably the inhibitor is a 5-HT receptor antagonist of formula I, II or III, ICI-685, ICI-715, ICI-735, ICI-824, ICI-846, ICI-847, ICI-848, ICI-849, ICI-890, ICI-894, ICI-953, ICI-954, Compound 36a, Compound 37b or Compound 35b. Without wishing to be bound by any particular theory, administration of a bolus dose mediates apoptosis of certain cells, such as, among others, an activated T cell or a cancerous B cell (such as, e.g., a multiple myeloma cell), such that repeated doses of the inhibitor is not necessary since the bolus mediates the death of memory, or other, cells that would otherwise mediate the immune response that would otherwise cause the transplanted cell or tissue to be rejected. This effect can be mediated by a localized concentration of a 5-HT receptor antagonist at the 5HTR1B receptor, which concentration is sufficient to inhibit transmission of the serotonin signal, thereby mediating cell death and/or inhibition of an immune response by the cell.

### Kits

The invention encompasses various kits relating to inhibiting the interaction of serotonin with a serotonin receptor because, as disclosed elsewhere herein, inhibiting this interaction in turn inhibits activation of an immune cell thereby inhibiting an immune response. Thus, in one aspect, the invention includes a kit for modulating an immune response in a mammal. The kit comprises an effective amount of an inhibitor of the interaction of serotonin with a serotonin receptor. Such an inhibitor includes, preferably, a serotonin receptor antagonist. And the kit further comprises an applicator and an instructional material for the use thereof.

Additionally, one skilled in the art would appreciate, based upon the disclosure provided herein, that the inhibitor can be a compound that does not cross the blood-brain barrier and is preferably water soluble. This is because, as more fully discussed elsewhere herein, it may be desirable to inhibit serotonin signaling in a non-neural cell, while not affecting such signaling in a neural cell, which would be protected beyond the blood-brain barrier.

In a specific embodiment, the kit of the present invention comprises a 5-HT receptor antagonist, an applicator, and an instructional material for the use thereof. In another embodiment, the kit can comprise a 5-HT receptor antagonist, such as those described elsewhere herein, a container holding the 5-HT receptor antagonist, and an instructional material. The skilled artisan can provide the applicator.

Preferably, the kit of the present invention comprises a 5-HT receptor antagonist of formula I, II or III, ICI-685, ICI-715, ICI-735, ICI-824, ICI-846, ICI-847, ICI-848, ICI-849, ICI-890, ICI-894, ICI-953, ICI-954, Compound 36a, Compound 37b or Compound 35b. Additionally, the kit can comprise an instructional material and an applicator for the administration of a 5-HT receptor antagonist of the present invention.

The kits of the present invention can be used to treat the diseases and conditions disclosed elsewhere herein. Specifically, the kits of the present invention can be used to treat, among other things, autoimmune diseases, such as psoriasis, organ transplant rejection, such as kidney transplant rejection, lymphoma, such as Hodgkin's lymphoma or non-Hodgkin's lymphoma, and B-cell neoplasias, such as multiple myeloma. The kits described in the present invention are not limited to the uses above however, and can be used in any method derived from the teachings disclosed herein.

Those skilled in the art will recognize, or be able to ascertain using no more than routine experimentation, numerous equivalents to the specific procedures, embodiments, claims, and examples described herein. Such equivalents were considered to be within the scope of this invention and covered by the claims appended hereto. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present application.

It is to be understood that wherever values and ranges are provided herein, all values and ranges encompassed by these values and ranges, are meant to be encompassed within the scope of the present invention. Moreover, all values that fall within these ranges, as well as the upper or lower limits of a range of values, are also contemplated by the present application.

The following examples further illustrate aspects of the present invention. However, they are in no way a limitation of the teachings or disclosure of the present invention as set forth herein.

### EXAMPLES

The invention is now described with reference to the following Examples. These Examples are provided for the purpose of illustration only, and the invention is not limited to these Examples, but rather encompasses all variations that are evident as a result of the teachings provided herein.

### Example 1

### Efficacy of 5-HT Receptor Antagonists in Cell Lines

### Cell Lines

Cell lines used in these studies were obtained from the American Type Culture Collection (ATCC; Manassas, Va.) or were otherwise obtained as indicated and were maintained under standard laboratory growth conditions. The neoplastic T-cell lines used in the studies included CCRF-CEM cells, a CD4+ lymphoblastic T-cell leukemia line (Foley et al., 1965, Cancer 18: 522-529). The B-cell neoplastic cell lines used were as follows: RPMI 8226 (a plasmacytoma derived from a multiple myeloma patient (Matsuoka, et al., 1967, Proc. Soc. Exp. Biol. Med. 125: 1246-1250), U266 (established from an IgE-secreting myeloma patient (Nilsson, et al., 1970, Clin. Exp. Immunol., 7: 477-489) and ARH77 (an EBV transformed plasma cell leukemia (Burk, et al., 1978, Cancer Res, 38: 2508-2513). The MM1S cells, a dexamethasone sensitive cell line derived from the MM1 cell clone, isolated from an IgA-secreting myeloma patient in the leukemic phase, (Goldman-Leikin, et al., 1989, Lab. Clin. Med., 113: 335-345), were a kind gift from Dr. Kenneth Anderson. BE(2)-C is a clone of the SK-N-BE(2) neuroblastoma cell line (see ATCC CRL-2271) that was established in November of 1972 from a bone marrow biopsy taken from child with disseminated neuroblastoma after repeated courses of chemotherapy and radiotherapy. BE(2)-C was deposited at the ATCC by June L. Biedler, Memorial Sloan-Kettering Cancer Center. The RPMI-Dox 40 cell line (Dalton and Salmon, 1992, Hematol. Oncol. Clin. North Am., 6: 383-393) and the RPMI-LR5 (Hideshema, et al., 2005, Proc. Nat'l. Acad. Sci. USA, 102: 8567-8572 are doxorubicin-resistant and melphalan-resistant multiple myeloma cell lines, respectively. Dexamethasone-sensitive (MM1S) and -resistant (MM1R) human multiple myeloma cell lines, as well as the dexamethasone-sensitive (OPM-2) and -resistant (OPM-1) multiple myeloma cell lines were used (Gomi, et al., 1990, Cancer Res. 50: 1873-1878). All multiple myeloma cell lines were cultured in RPMI medium 1640 containing 10% FBS (Sigma, St. Louis, Mo.), 2 μM L-glutamine, 100 units/ml penicillin, and 100 μg/ml streptomycin (Gibco, La Jolla, Calif.).

Primary multiple myeloma patient plasma cells were purified from bone marrow aspirates by negative selection by using an antibody mixture (RosetteSep Separation System, StemCell Technologies, Vancouver) as described in Hideshima, et al., (2003, Blood 101: 1530-1534). The purity of MM cells was >90%, as confirmed by flow cytometric analysis using anti-CD138 Ab (Pharmingen, San Jose, Calif.).

### [³H]-Thymidine Incorporation Assays

Cells were harvested from culture media and washed three times in 20 mL room temperature Hanks Balanced Salt Solution (HBSS) by centrifugation. Cells were plated in 96-well plates (Corning-Costar, Acton, Mass.) at a density of 5×10⁴ cells per 180 μL complete growth media. Following addition of cells, test agents were added to culture wells in a volume not exceeding 20 μL for aqueous vehicle or a 0.05% final concentration of DMSO vehicle. Untreated samples contained an equivalent concentration of vehicle as a control. Proliferation assays were carried out for the time indicated following drug addition and pulsed with 1 μCi [³H]-thymidine (NEN-Life Sciences, Boston, Mass.) during the final 6 hours of culture. At the completion of the assay, cells were harvested on glass fiber filters using a PHD harvester (Brandel, Gaithersburg, Md.). Filters were soaked overnight in 3 mL CytoScint scintillation fluid (ICN Biomedicals, Irvine, Calif.) and counted using a β-counter (Becton Dickinson, San Jose, Calif. All samples were performed in at least triplicate.

### Colorimetric MTT Assays for Cell Viability

Cells were harvested and treated with the indicated concentrations of drug as described for [³H]-thymidine incorporation assays and trypan-blue exclusion studies, except that the volume contained in each well was reduced to 100 μL. Assays were carried out for the indicated time following drug addition. Prior to the completion of assays, 50 mg MTT reagent (3-(4,5-dimethylthiazon-2-ly)-2,5-diphenyl tetrasodium bromide) was dissolved in 10 mL PBS, pH 7.4, as per the manufacture's directions. At the completion of assays, 10 μL dissolved MTT reagent was added to each well, mixed by gentle agitation and incubated at 37° C. in tissue culture incubators for 4 hours. 100 μL isopropanol/0.04N HCl was added to each well and mixed thoroughly by repeated pipetting. Absorbance was measured using an ELISA plate reader at wavelength of 570 nm. All samples were plated in at least quadruplicate for MTT assays.

### Trypan Blue Exclusion Studies

Cells were harvested and treated with indicated concentrations of drug as described above for [³H]-thymidine incorporation assays. Assays were carried out for the indicated number of hours following drug addition. At the completion of the assay cells were harvested from 96-well plates and washed and re-suspended in HBSS. Cell suspensions were then stained with a 1:2 dilution of 0.4% (w/v) trypan-blue solution for approximately 15 minutes. Viable cells (un-stained with trypan-blue) were enumerated using a hemocytometer.

### Assessing Apoptosis by Annexin V Binding

Cells are harvested, washed twice in cold PBS (4° C.) and resuspended at a concentration of 1×10⁶ cells/ml in binding buffer (10X; 0.1M HEPES/NaOH, pH 7.4; 140 mM NaCl; 25 mM CaCl₂). Cells (100 μl) are aliquoted into FACS tubes and Annexin V die is added. Tubes are mixed gently and incubated at room temperature for 15 minutes in the dark. Binding buffer (400 μl) is added to each tube and analyzed via flow cytometry.

The results of the experiments presented in this Example are now described.

The MTT assay was employed for measuring cellular proliferation, or lack thereof, in several lines, including several strains of multiple myeloma cells. MTT assays measure the amount of yellow MTT (3-(4,5-Dimethylthiazol-2-yl)-2,5-diphenyltetrazolium bromide) reduced to purple formazan when mitochondrial reductase enzymes are active, thus directly measuring the number of viable cells (Mosmann, 1993, J. Immunol. Meth., 65: 55-63). The production of formazan in cells treated with a 5-HT receptor antagonist was measured relative to the production in control cells, and a dose-response curve was generated.

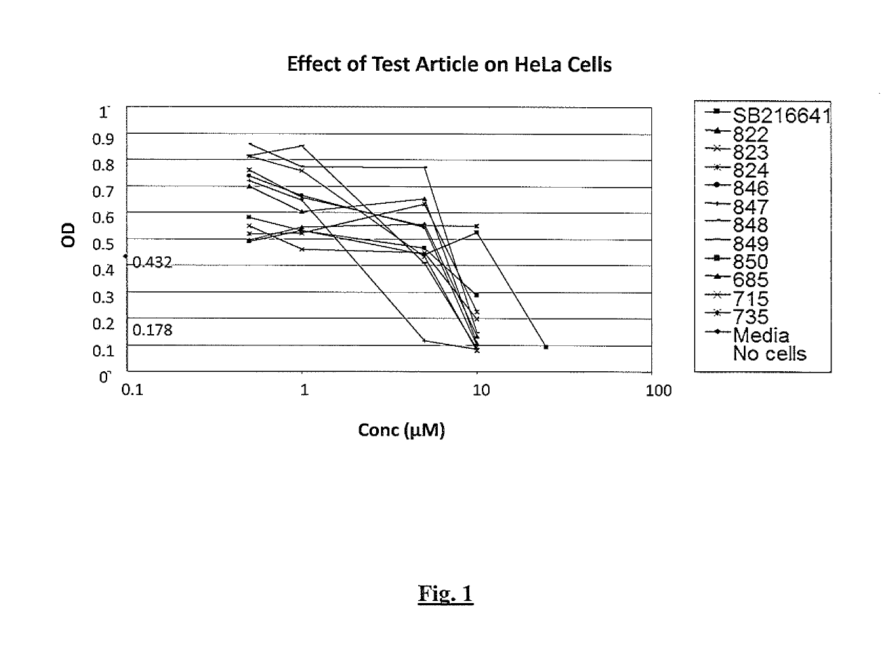

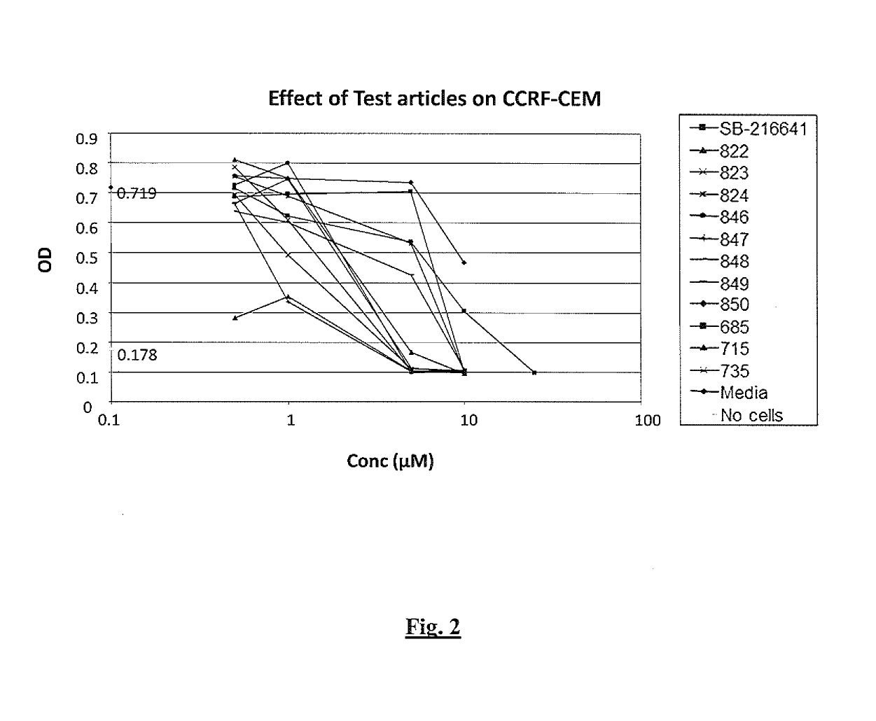

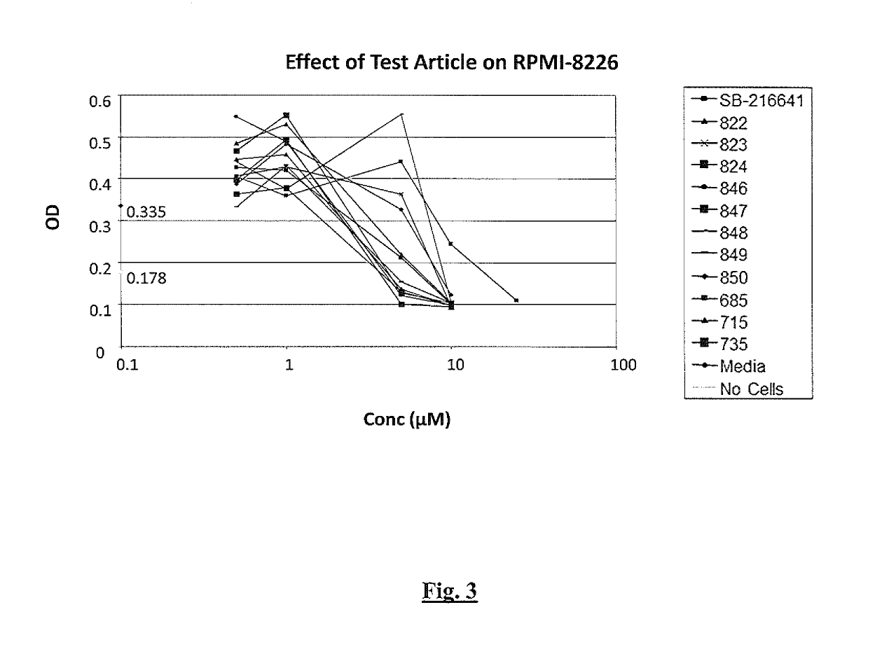

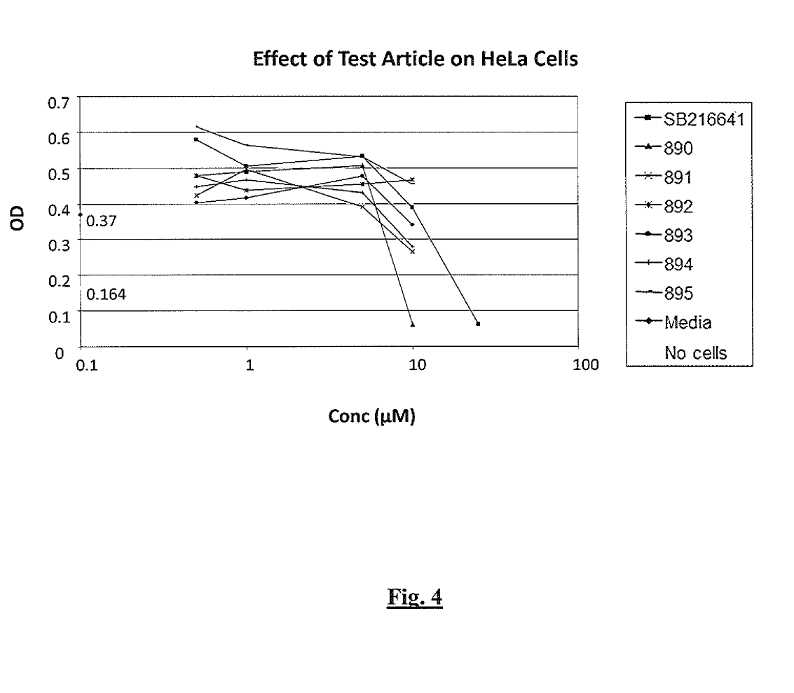

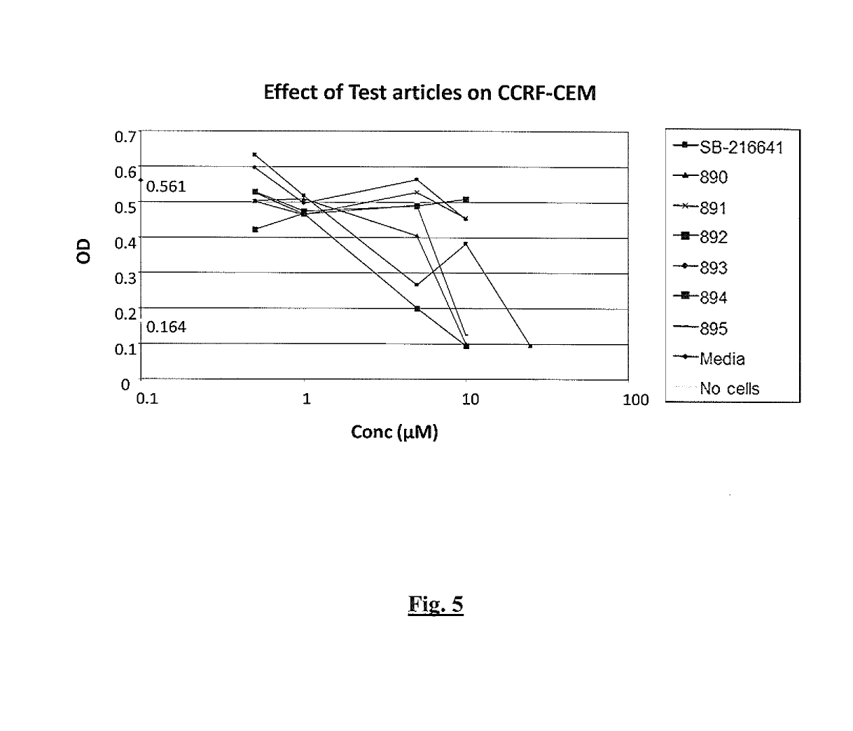

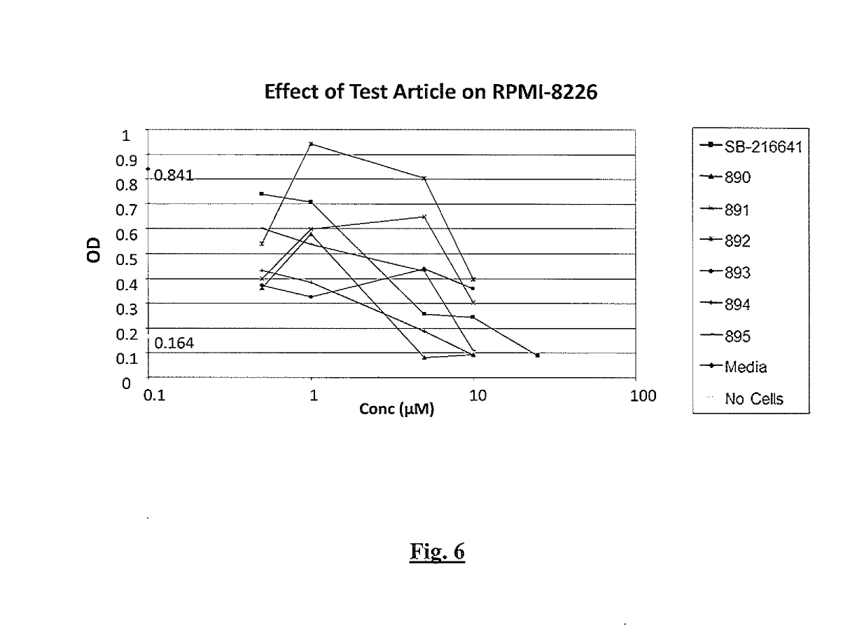

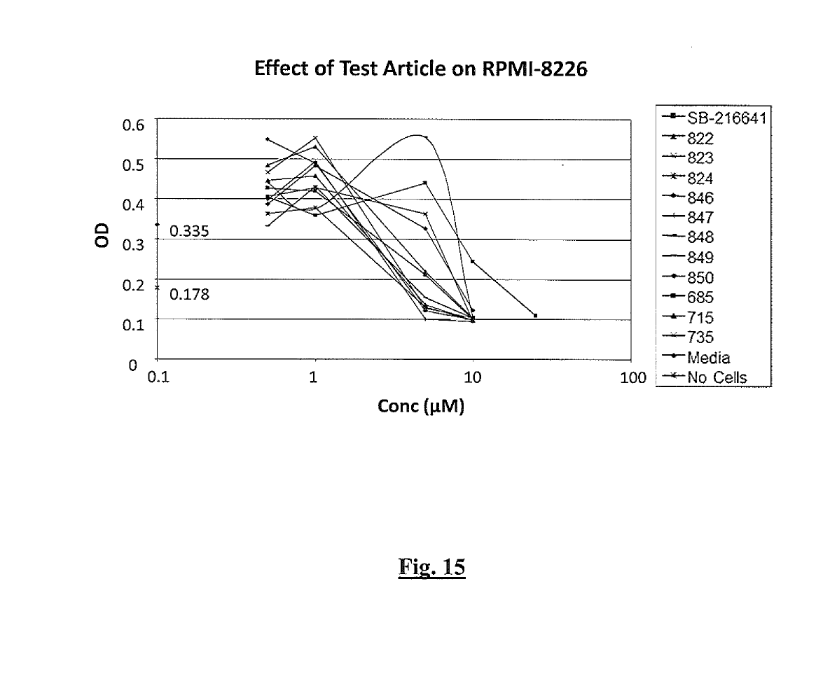

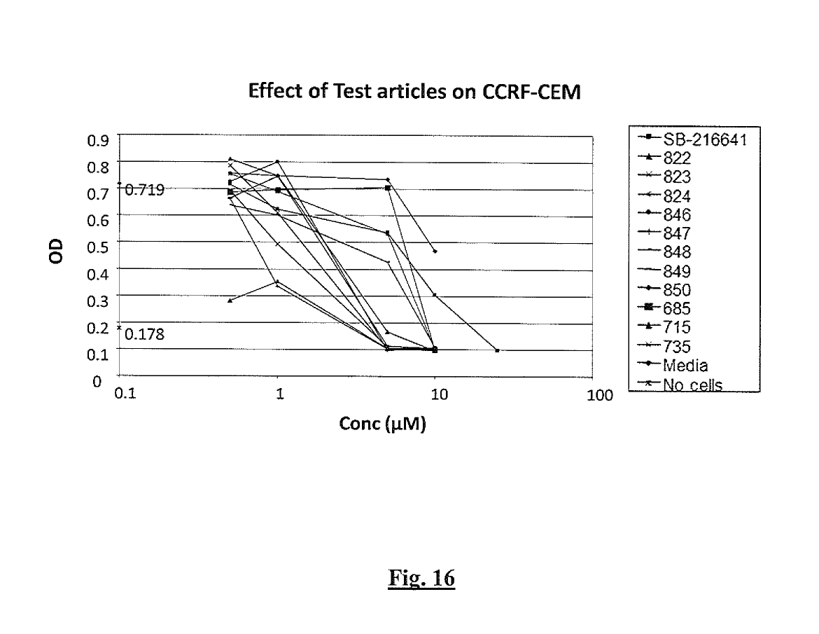

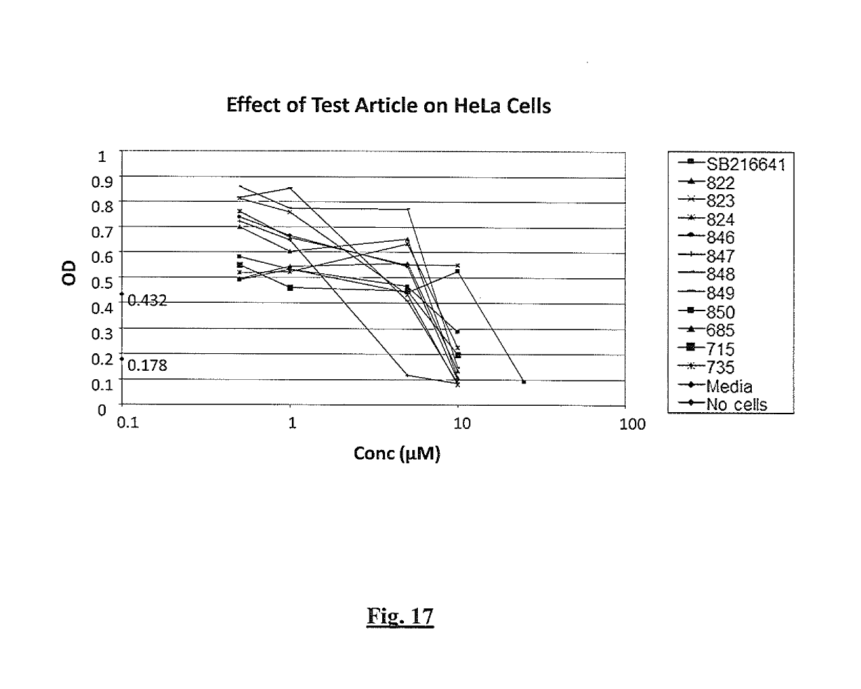

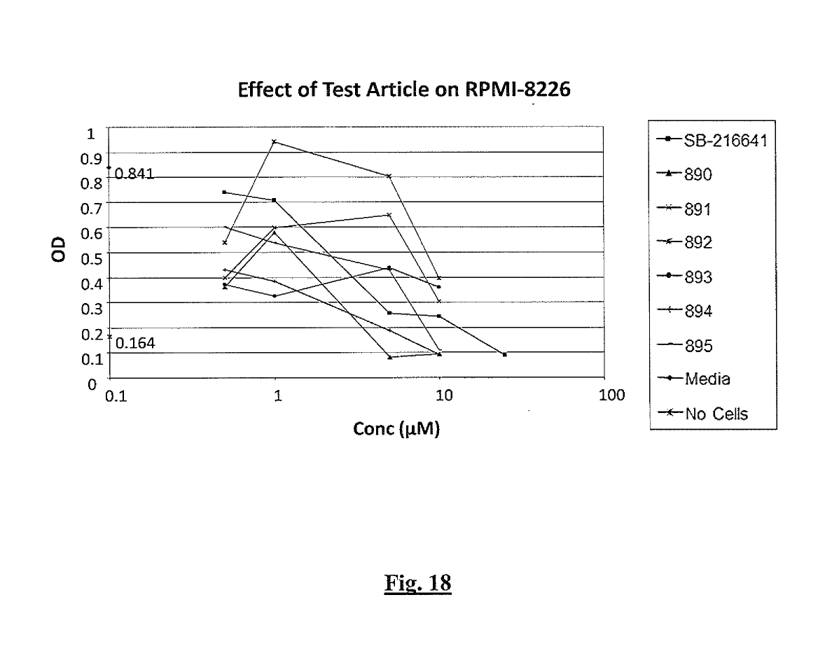

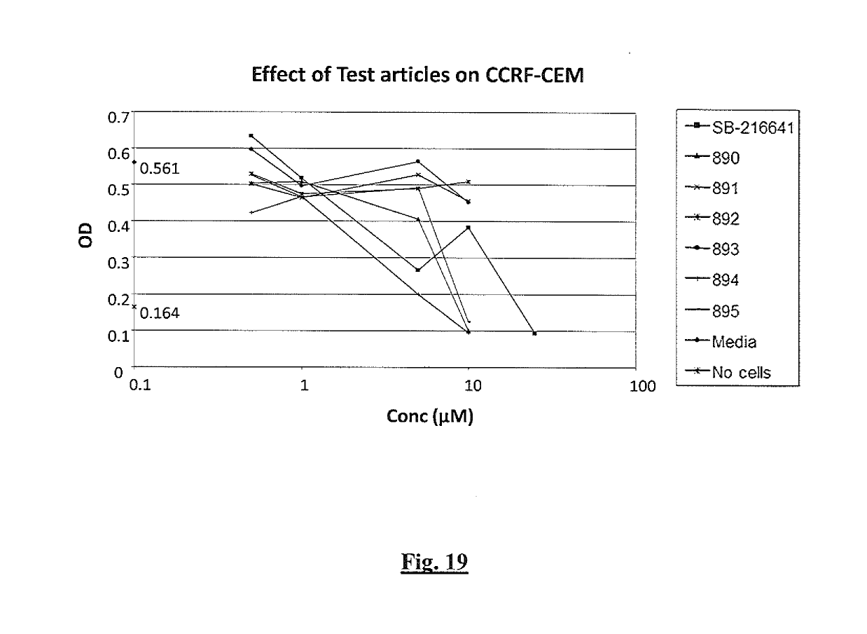

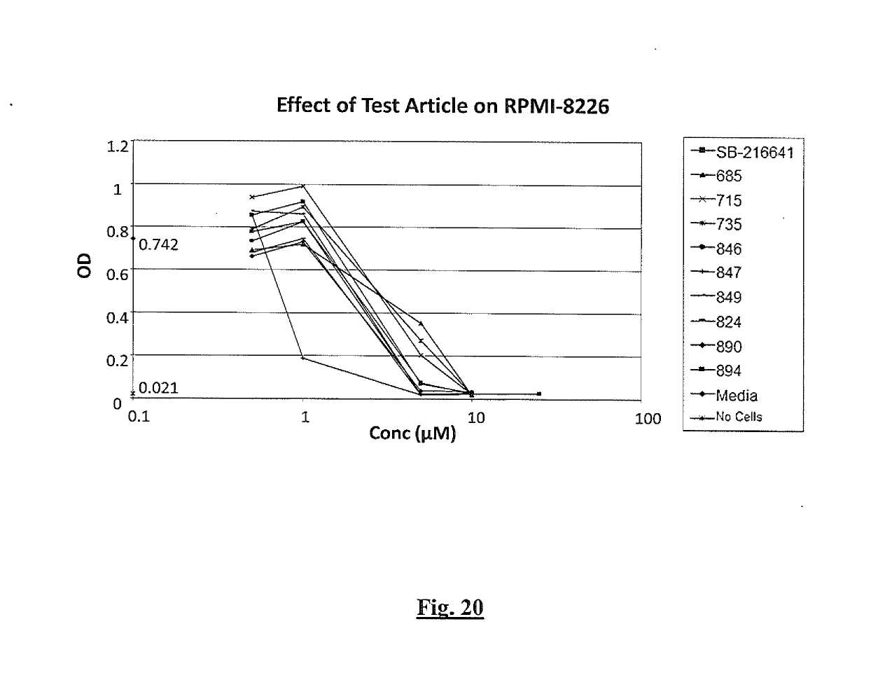

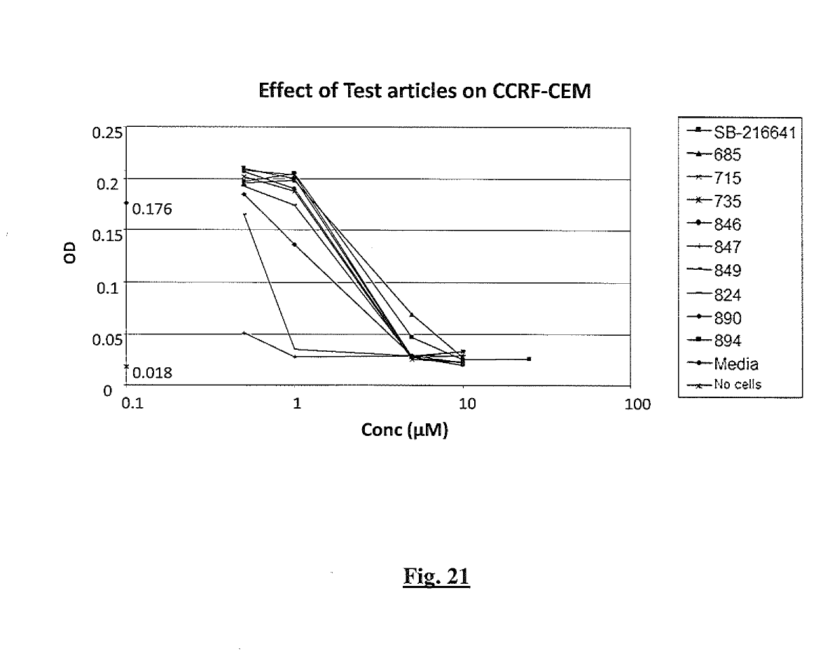

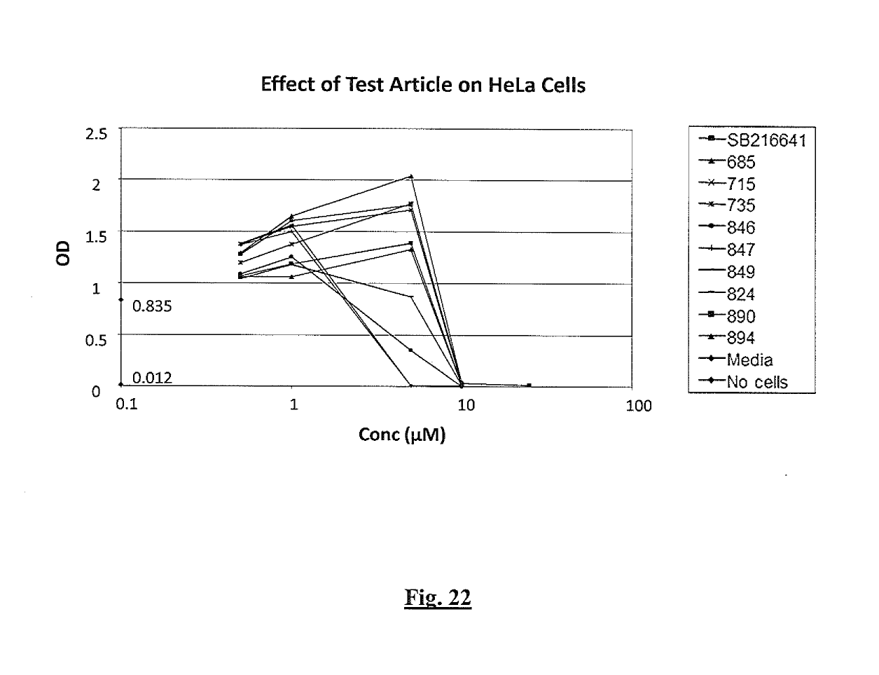

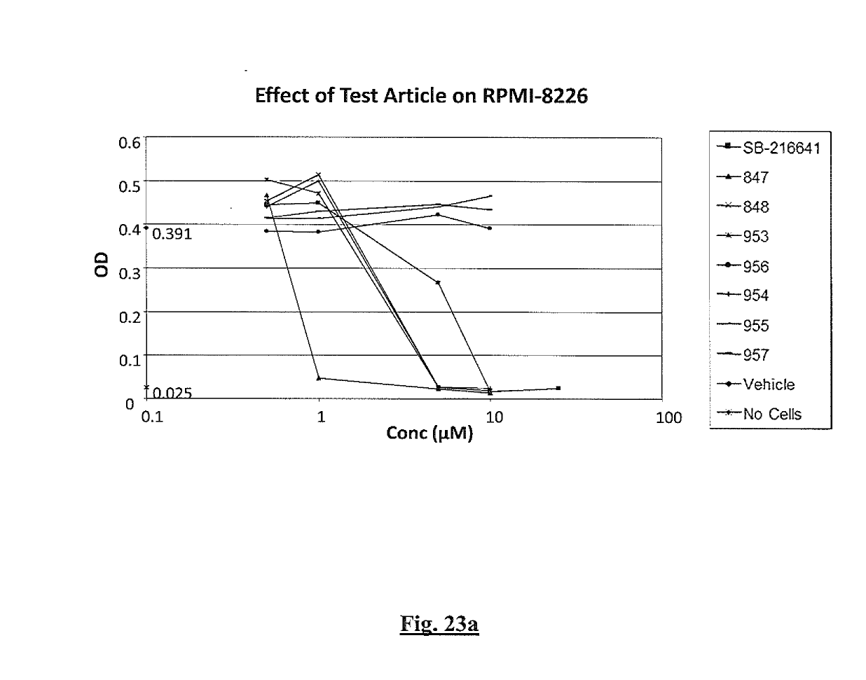

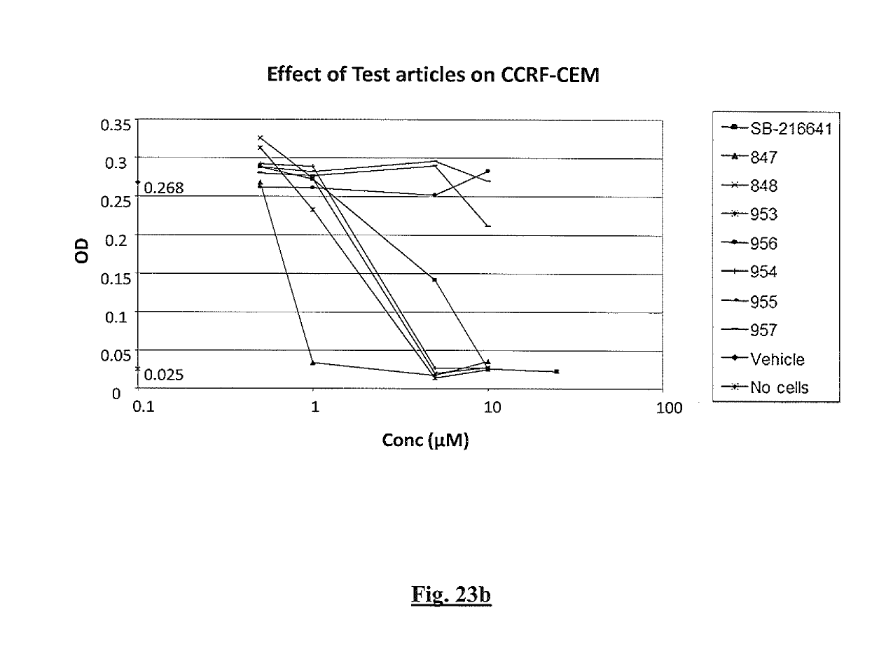

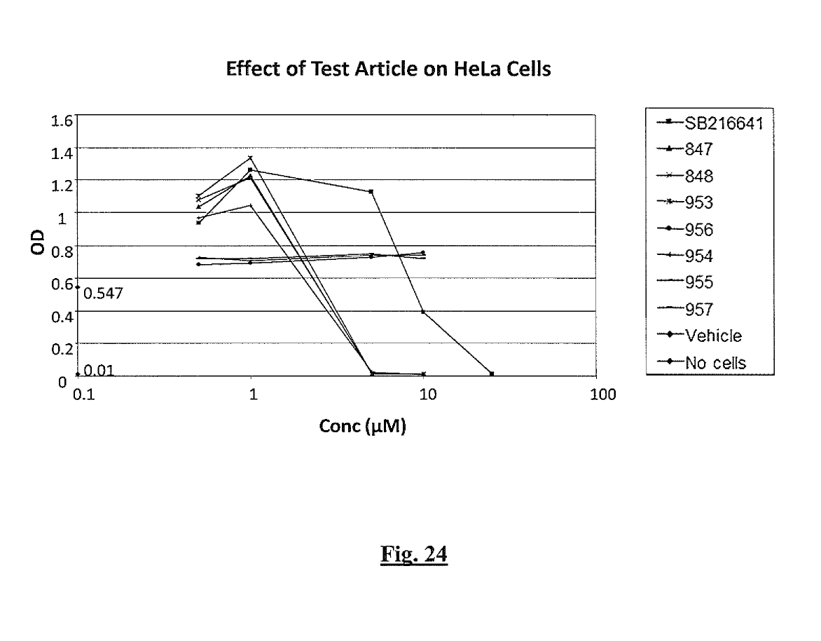

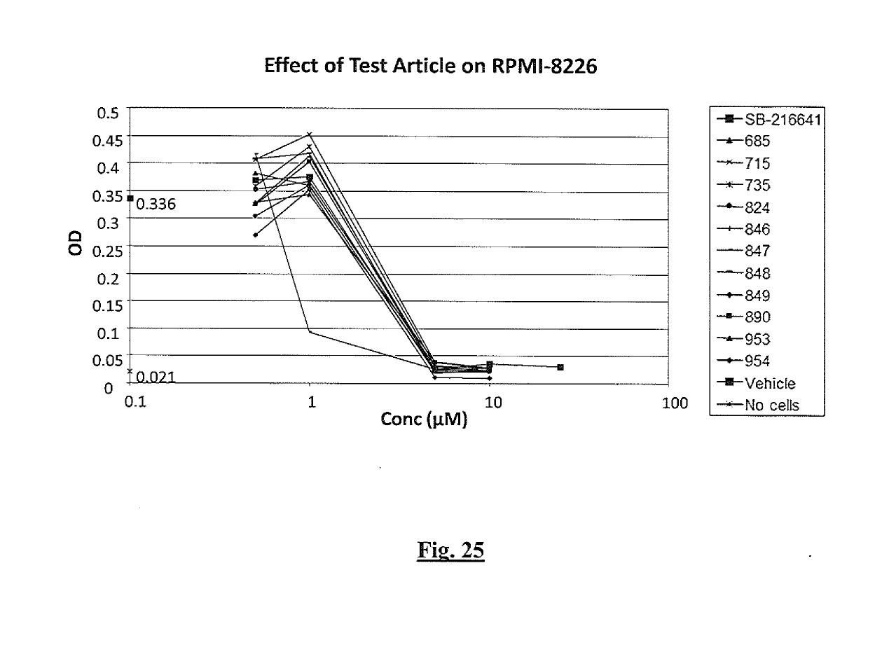

HeLa cells, the T cell lymphoma line CCRF-CEM, and the multiple myeloma cell line RPMI-8226 were treated with the selective 5-HT1B antagonists SB 216641, ICI-822, ICI-823, ICI-824, ICI-846, ICI-847, ICI-848, ICI-849, ICI-850, ICI-685, ICI-715, ICI-735, ICI-890, ICI-891, ICI-892, ICI-893, ICI-894, ICI-895, ICI-953, ICI-956, ICI-954, ICI, 955 and ICI-957 and cell viability and proliferation were then measured using an MTT assay (FIGS. 1-6 and 15-25). Loss of viability and proliferation inhibition were pronounced in the T cell and multiple myeloma cell lines (FIGS. 2-3, 5-6, 15-16, 18-21, and 23-25) compared to HeLa cells.

### Treatment of Arthritis Using Compounds of the Invention

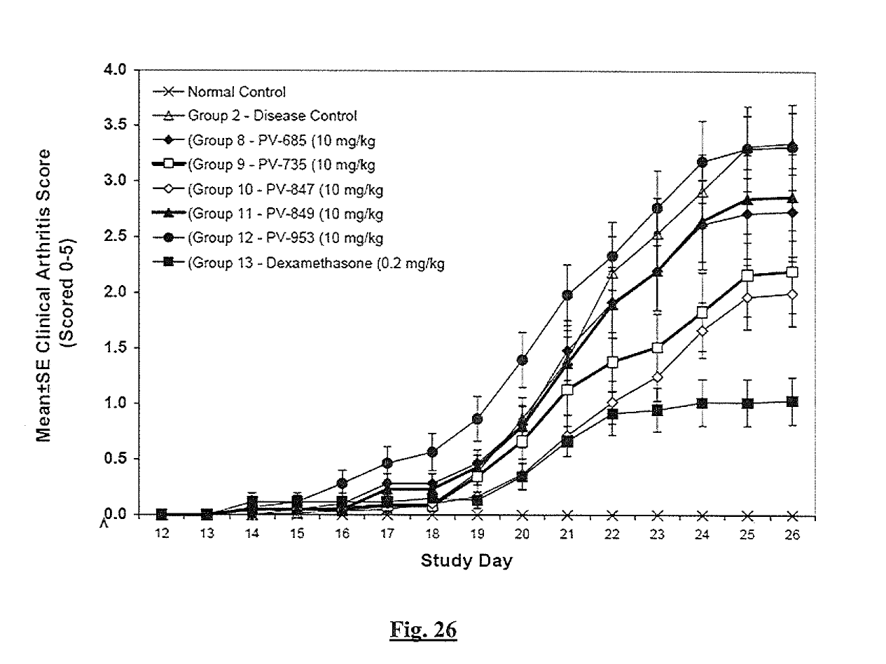

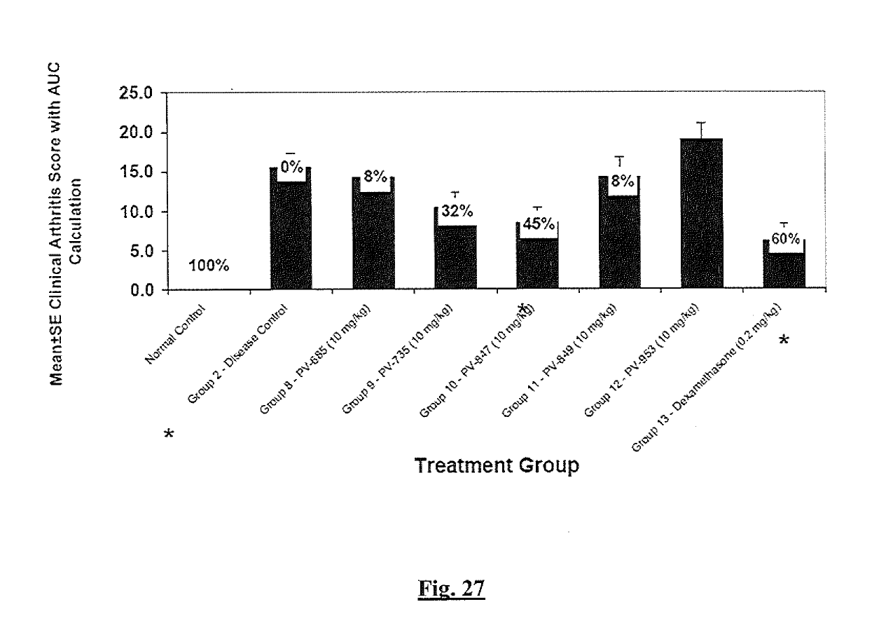

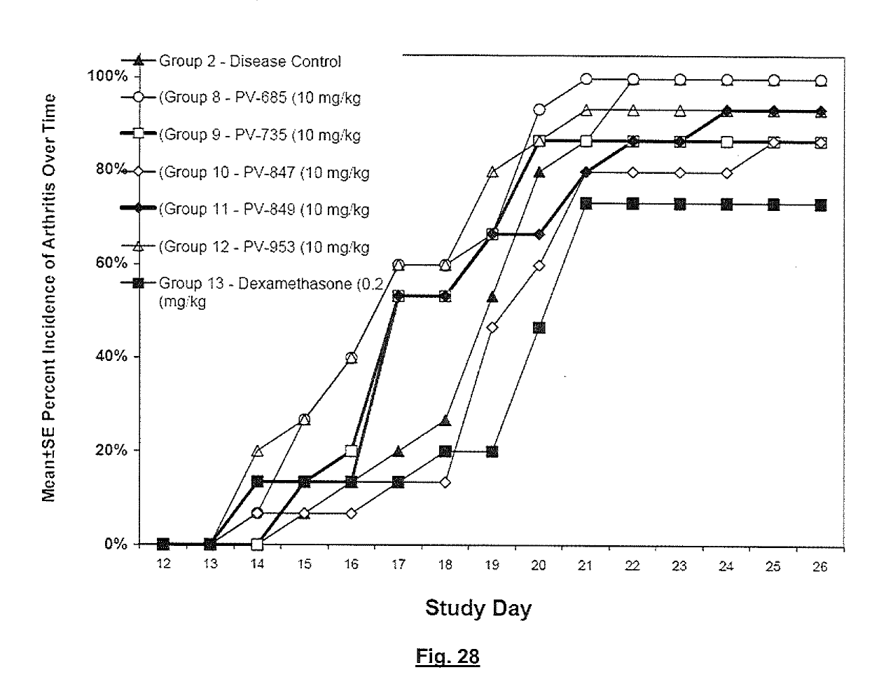

FIGS. 26-28 illustrate the effect of various compounds of the invention on the clinical arthritis score of mice treated with various compounds of the invention. Notably, FIG. 26 illustrates that ICI-847, delivered orally at 10 mg/kg daily, for about 21 days, is as effective as dexamethasone in mice sensitized with collagen injections, resulting in the same clinical arthritis score as dexamehtasone, a standard rheumatoid arthritis (RA) animal model.

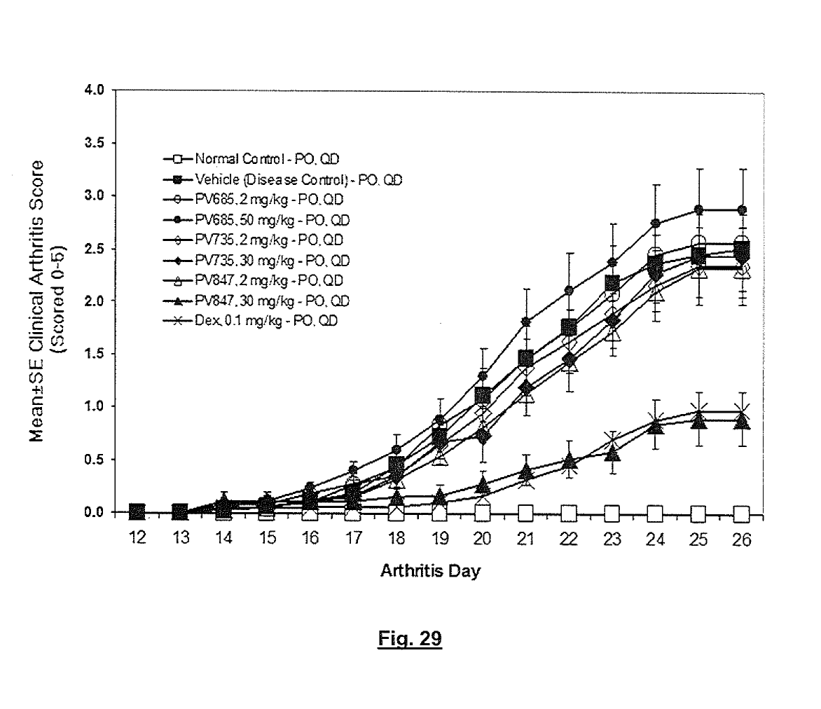

Additional testing of the effectiveness of compounds ICI-685, ICI-735 and ICI-847 at higher and lower concentrations were conducted, as illustrated in FIG. 29. These data illustrate the effectiveness of ICI-847 at treating rheumatoid arthritis (RA), and the symptoms of RA, and illustrates that at 30 mg/kg, ICI-847 is as effective for the length of the study as is dexamethasone.

Dexamethasone is a well-known compound used for treatment of arthritis, among other inflammatory diseases. The present experimental results therefore suggest that compounds of the invention can be useful for treating arthritis and related conditions.

### Treatment of Asthma Using Compounds of the Invention

Compounds of the invention were also tested for efficiacy in an asthma model. Table 1 demonstrates the efficacy of compounds of the invention in treating asthma in an asthma model. Table 1 illustrates that both ICI-847 and ICI-735, delivered intraperitoneally at a dose of 20 mg/ml, were at least as effective as dexamethasone at decreasing lung resistance in an Aspergillus fumigatus-based mouse asthma model.

A cohort of mice was administered intraperitoneal injections of Aspergillus fumigatus on days 0, 14, 26, 27, and 28. Lung resistance was tested on day 29 immediately following a tracheotomy/methacholine procedure. Animals receiving dexamethasone were administered dexamethasone on days 26, 27 and 28. Animals receiving a compound of the invention were administered the compound just hours before the lung resistance test. As a control, all test compounds, as well as dexamethasone, were also administered to mice in the absence of an A. fumigatus insult. All animals treated with compound but not with A. fumigatus had a baseline value of about 3 in the lung resistance test.

Dexamethasone is a well-known compound used for treatment of asthma, among other inflammatory diseases. The present experimental results therefore suggest that compounds of the invention can be useful for treating asthma and related conditions.

TABLE 1
Treatment of Asthma Mouse Model
using Compounds of the Invention

| | Concentration | Lung resistance |
|---|---|---|
| Compound | (mg/ml) | (cm H₂O/ml/s) |
| ICI-847 | 20 | 7.25 |
| ICI-735 | 20 | 8.5 |
| ICI-685 | 20 | 12 |
| Dexamethasone | 20 | 7.75 |
| A. fumigatus insult, | 0 | 13.5 |
| no therapeutic compound | | |
| No A. fumigatus insult | 0 | 3 |

### Compounds and Synthesis

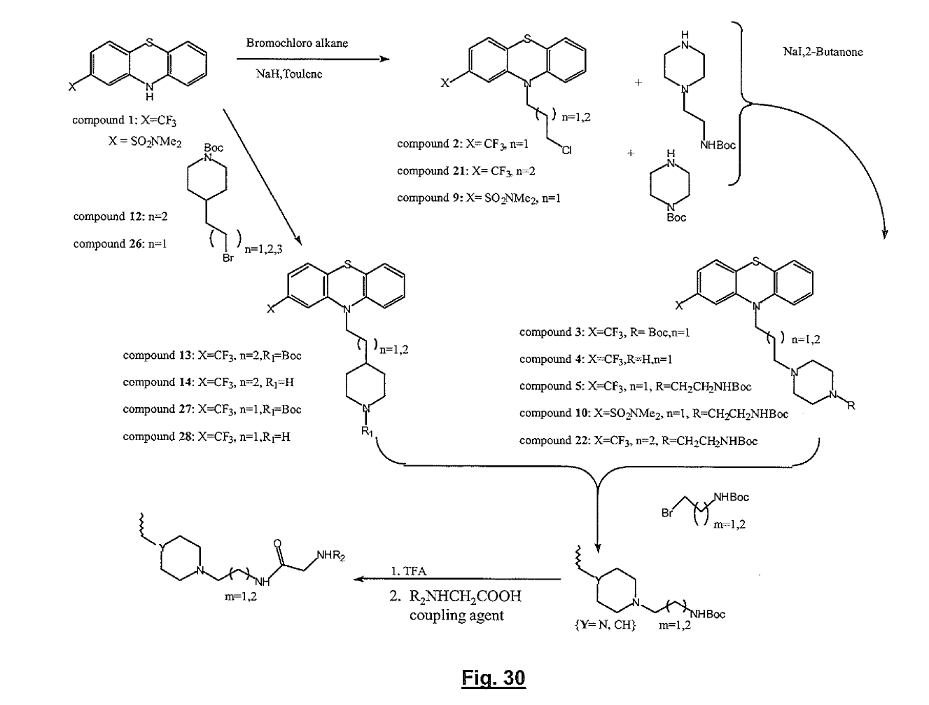

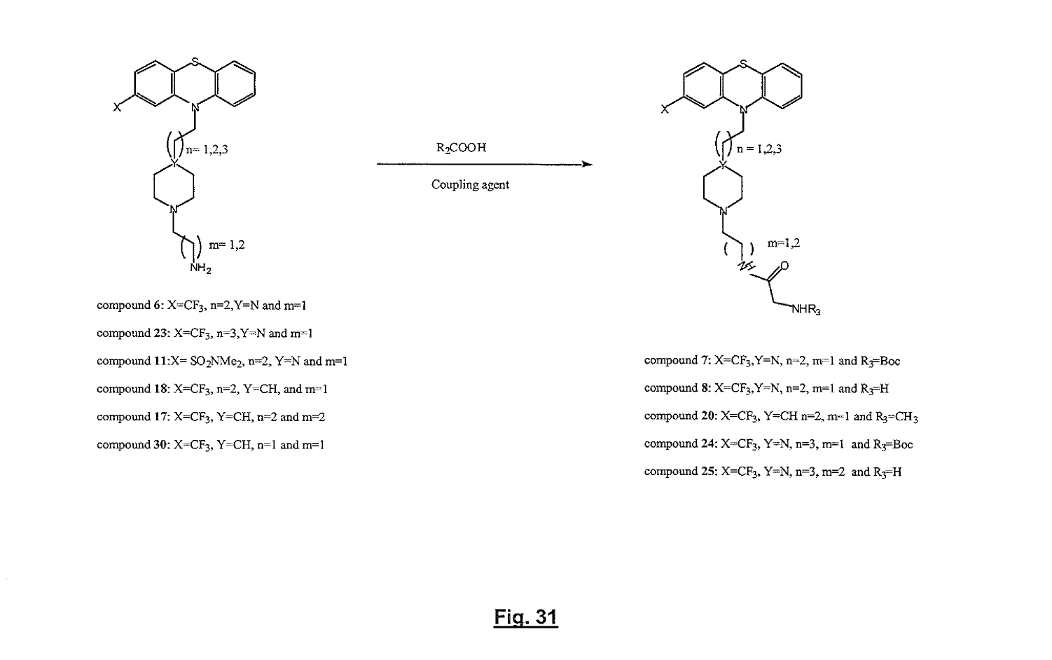

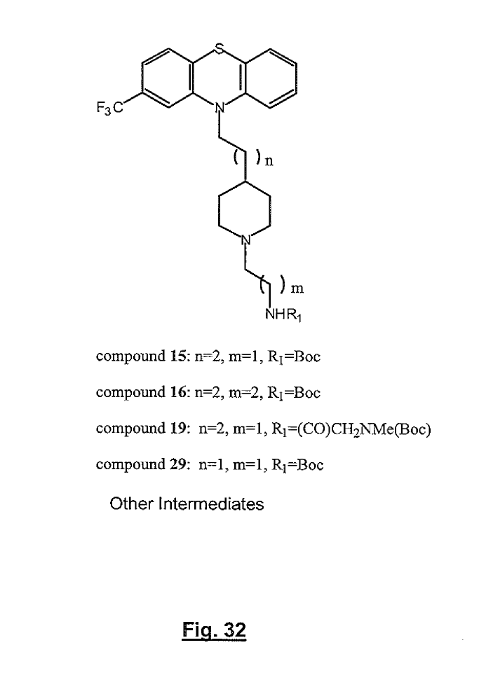

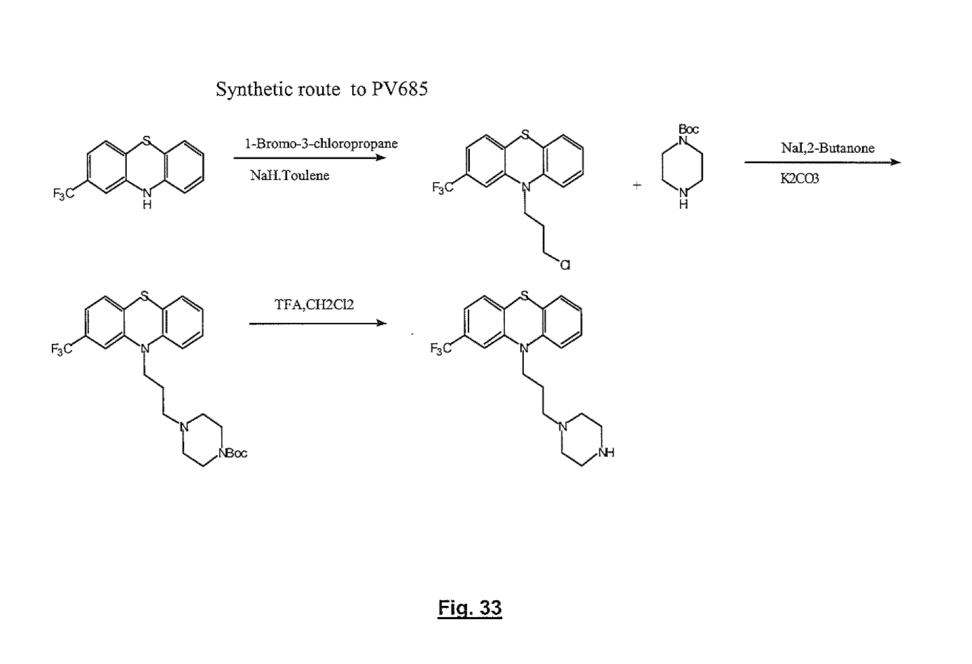

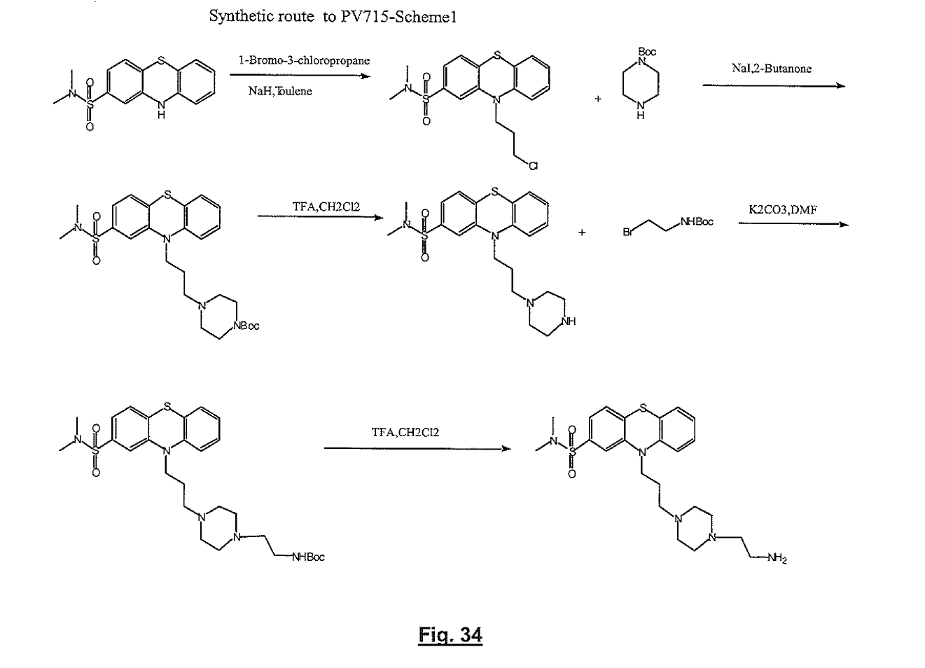

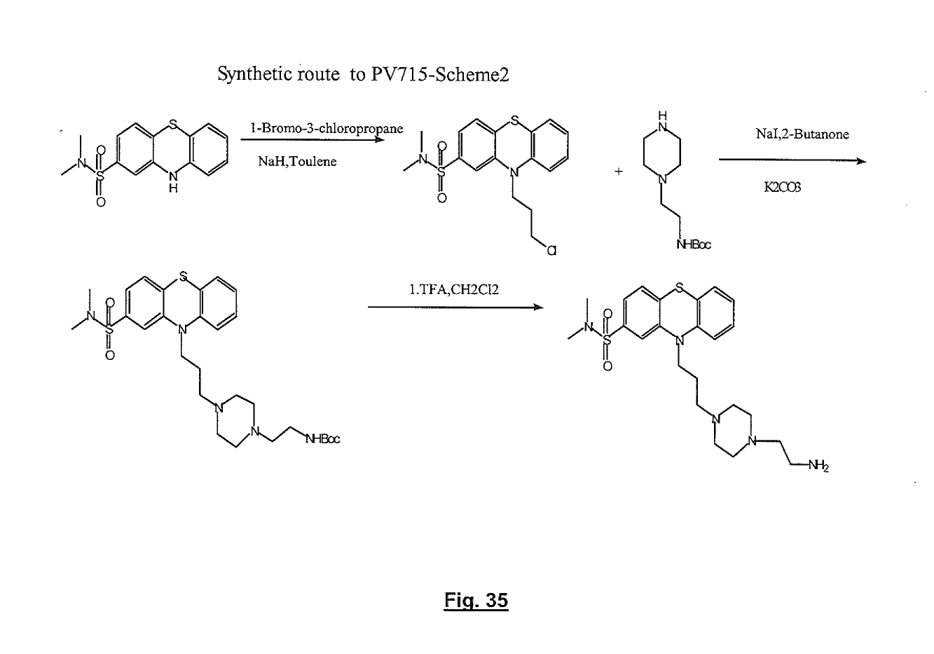

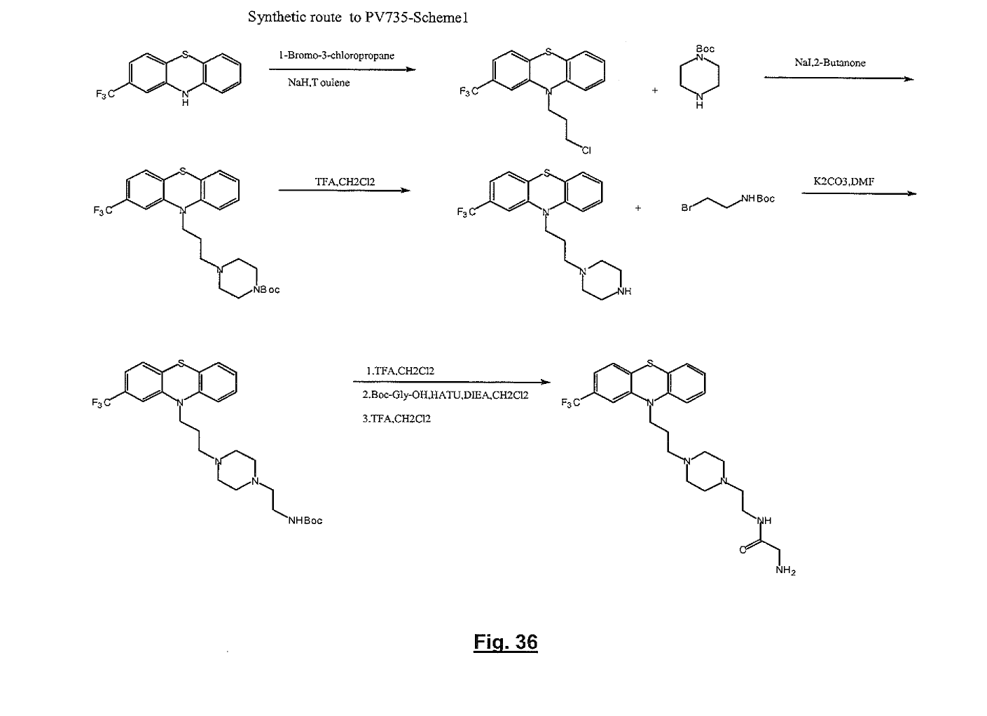

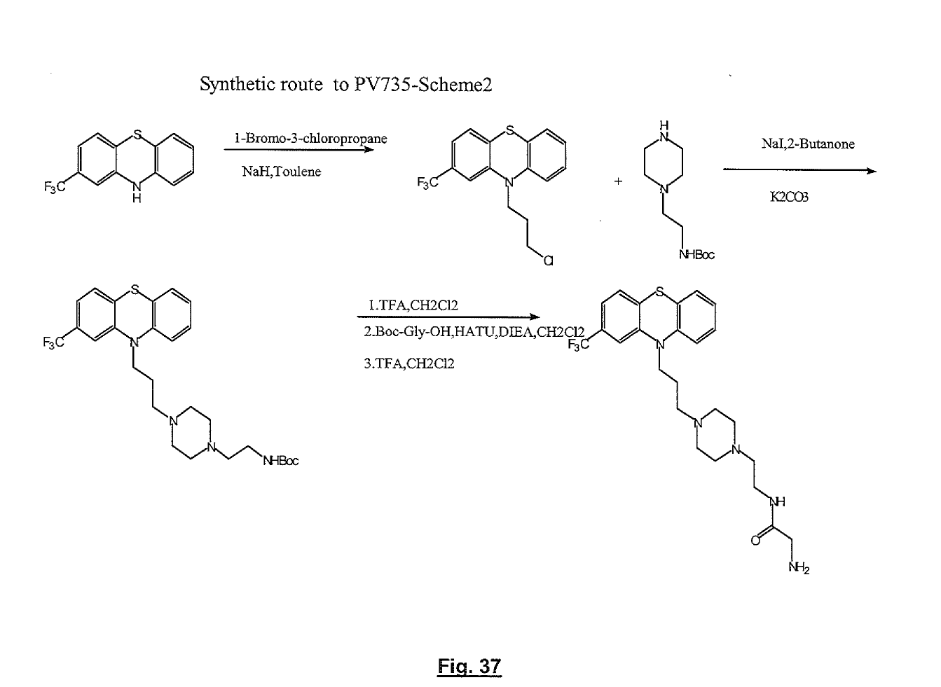

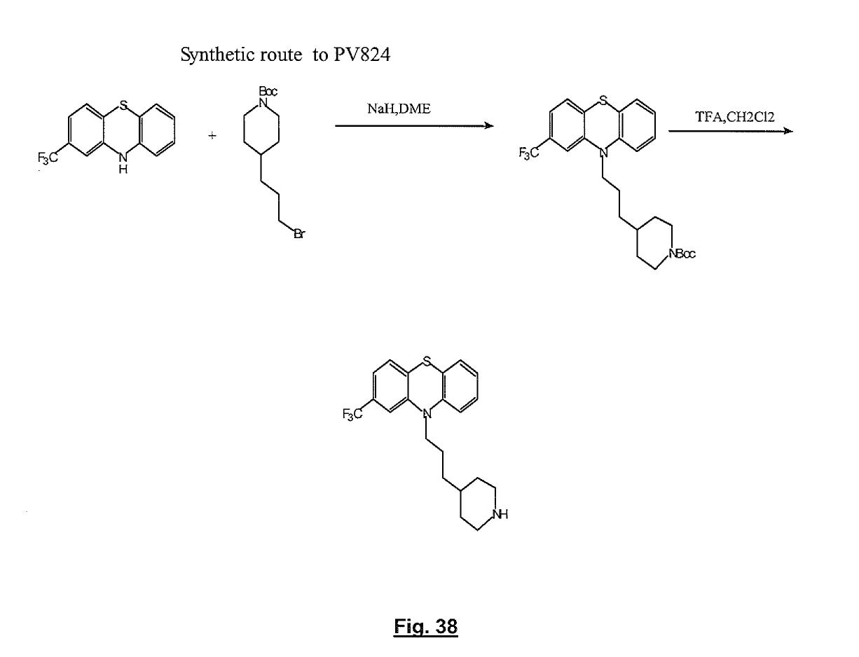

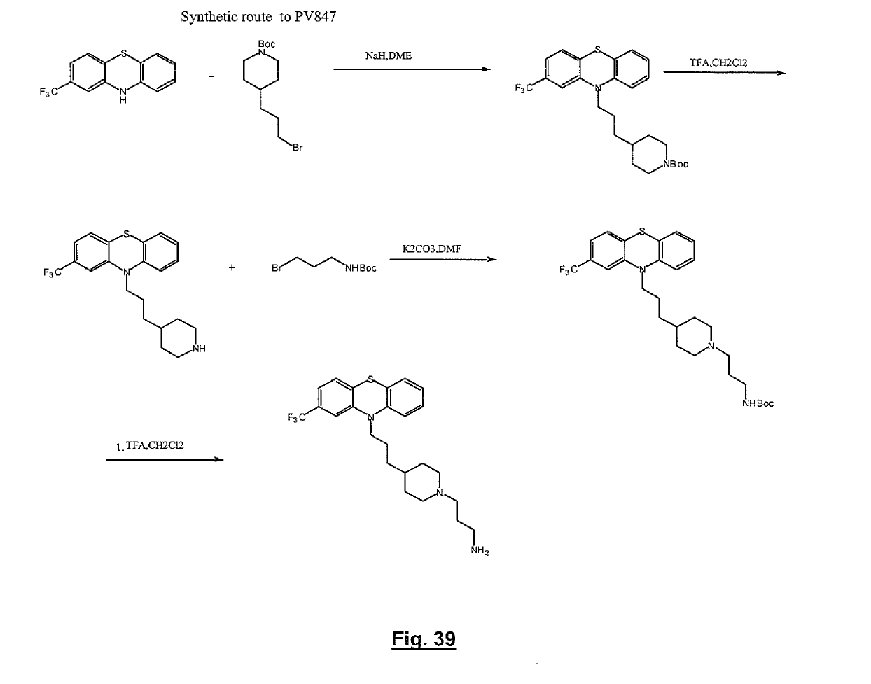

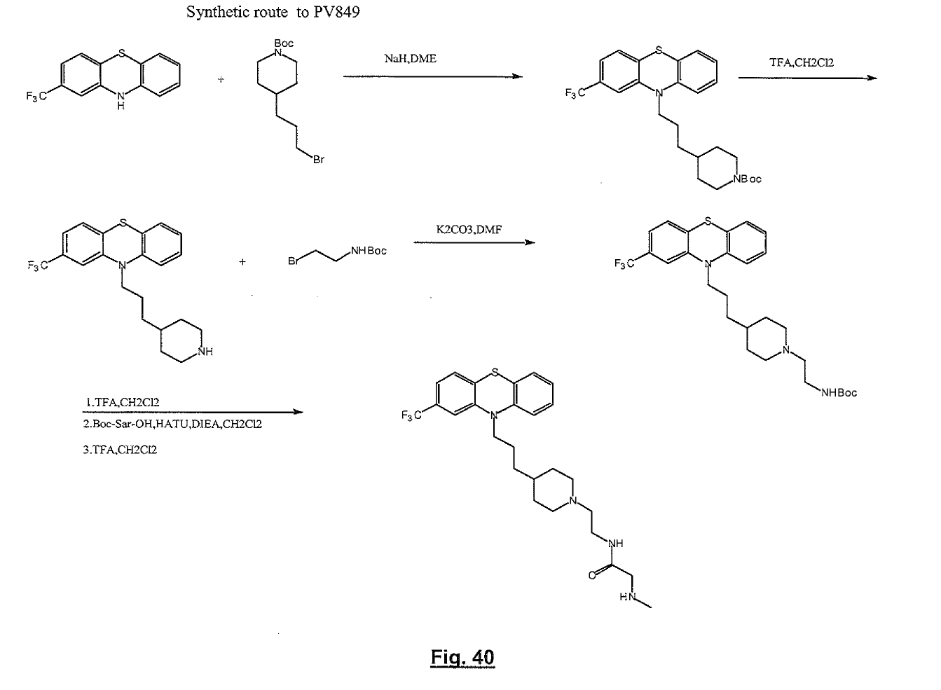

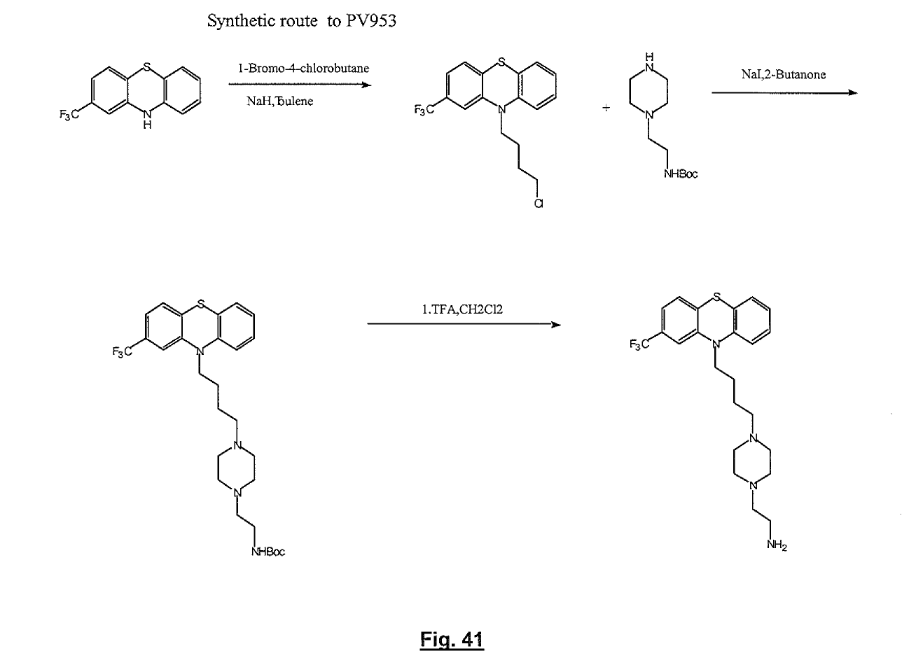

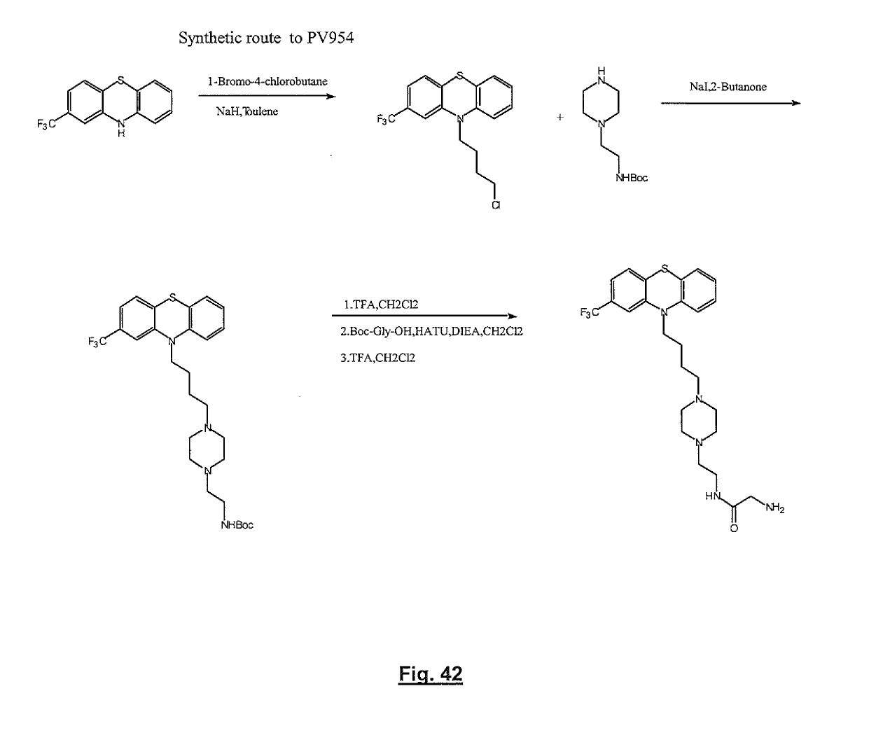

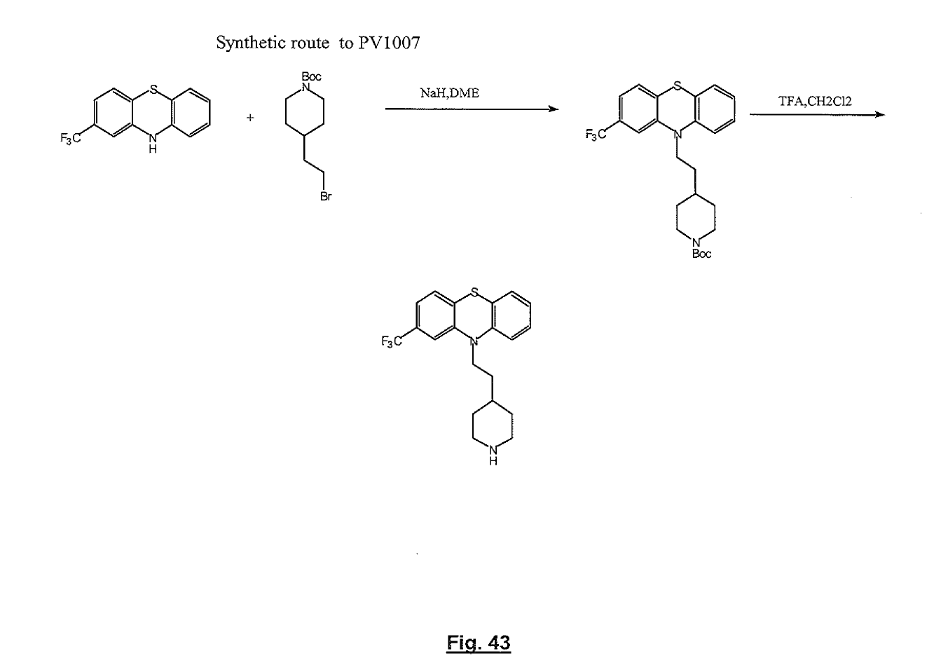

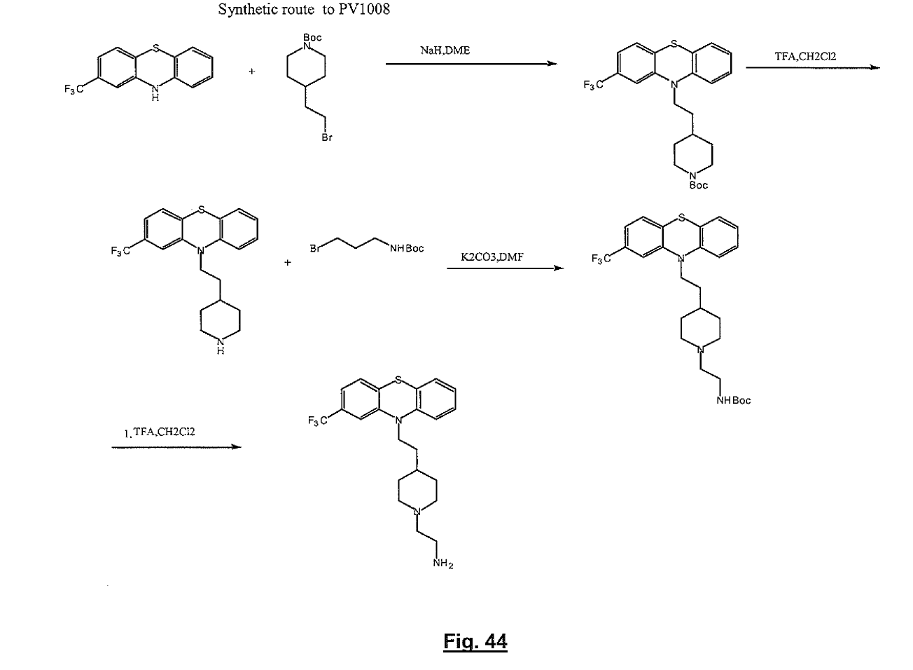

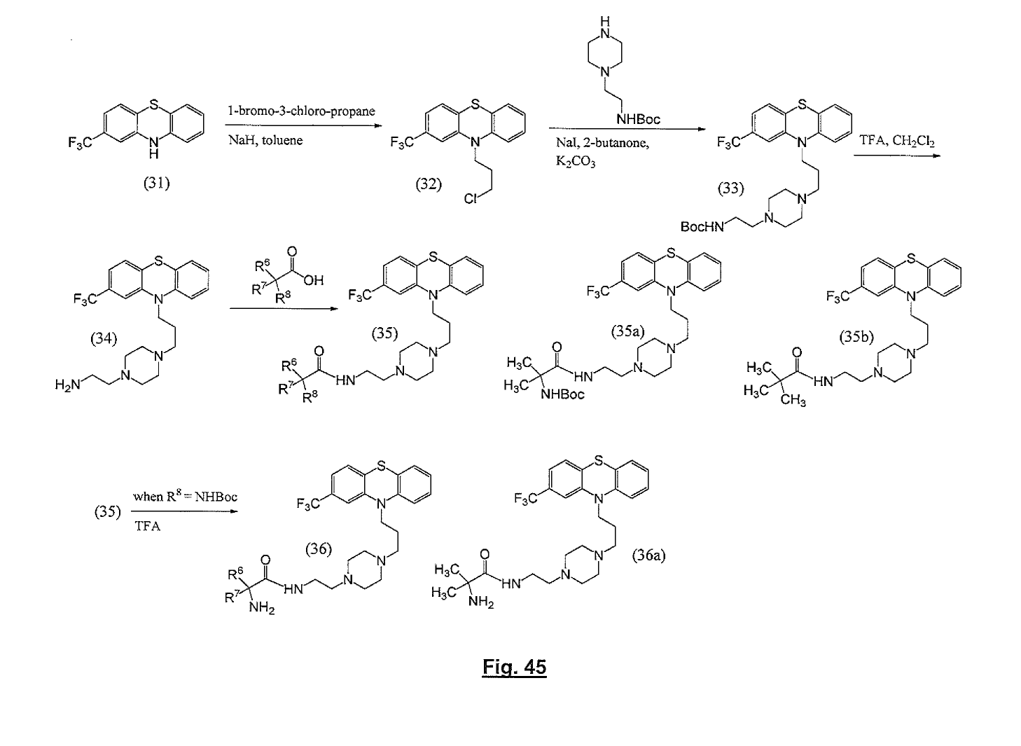

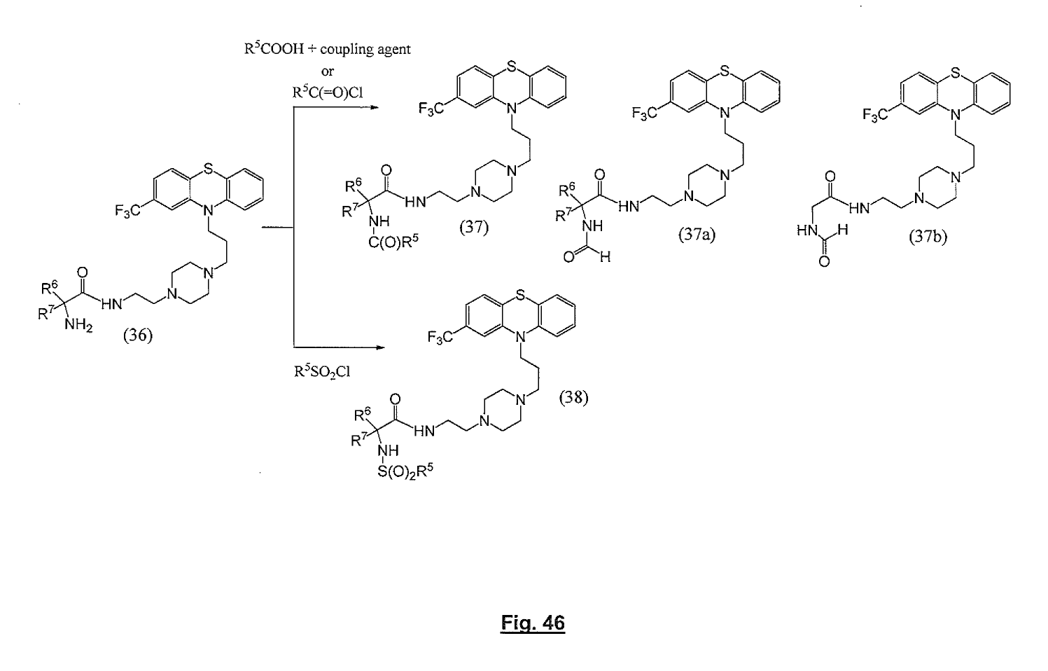

Following is a synthesis of a number of compounds according to the invention, some of which compounds are set forth in the synthetic schemes illustrated in FIGS. 30-32. Furthermore, each of FIGS. 33-46 provides a detailed synthetic pathway for a subset of compounds of the invention.

### Example 2

### 10-(3-Chloropropyl)-2-trifluoromethylphenothiazin (Compound 2)

To a stirred solution of 2-trifluoromethyl phenothiazine (compound 1) (2 g, 7.49 mmol) and sodium hydride (0.5 g, 10.42 mmol) in dry toluene (30 mL) was added 1-bromo-3-chloropropane (1.57 g, 10 mmol). The reaction mixture was stirred for 18 hours at 110° C. under an atmosphere of argon. The solution was cooled to room temperature and poured into an ice-water mixture, the crude product was extracted with ethyl acetate (3×50 mL) and the combined organic phase dried over anhydrous sodium sulphate. Final purification was performed by column chromatography (9:1 hexane:ethyl acetate) on silica gel to give 10-(3-chloropropyl)-2-trifluoromethylphenothiazine (1.5 g, 58%) as a solid.

### Example 3

### 10-[3-(4-N-Boc-1-piperazinyl)propyl)]-2-trifluoromethylphenothiazine Compound 3)

To a stirred solution of chloro compound 2 (2.57 g, 7.5 mmol) and 1-Boc-piperazine (1.4 g, 7.5 mmol) in methyl ethyl ketone (40 mL) was added sodium iodide (1.5 g, 10 mmol). The reaction mixture was stirred for 24 h at reflux under an atmosphere of argon. The reaction mixture was filtered and the filtrate concentrated under vacuum. The residue was partitioned between ethyl acetate (100 mL) and brine (50 mL). The organic layer was dried over anhydrous sodium sulphate, filtered and evaporated. The resulting residue was purified by silica gel column chromatography (9:1 CH₂Cl₂:MeOH) to give Compound 3 (2.7 g, 73%) as a solid. MS (ESI): m/z 494 (M+H).

### Example 4

### ICI-685 (Compound 4)

Compound 3 (750 mg, 1.52 mmol) was dissolved in dry CH₂Cl₂ (10 mL) and TFA (0.75 mL, 6.57 mmol) was added dropwise to this solution at 0° C. The solution was stirred at room temperature overnight. The reaction mixture was evaporated and the residue was purified by reversed phase HPLC on a C18 column (acetonitrile:water:TFA, gradient elution) to give the desired product (650 mg, 69%) as a white solid after lyophilization. MS (ESI): m/z 394 (M+H).

### Example 5

### 10-{3-[4-(N-Boc-2-amino)ethylpiperazinyl]propyl}-2-trifluoromethyl-phenothiazine (Compound 5)

To a stirred suspension of the chloropropyl derivative 2 (1.2 g, 3.5 mmol), potassium carbonate (1.5 g, 10.86 mmol), 1-(2-N-Boc-aminoethyl)piperazine (0.78 g, 3.5 mmol) in methyl ethyl ketone (30 mL), was added sodium Iodide (0.9 g, 6 mmol). The reaction mixture was stirred for 24 h at reflux under an atmosphere of argon. The reaction mixture was filtered, and filtrate was concentrated under vacuum. The residue was partitioned between ethyl acetate (30 mL) and brine (15 mL). The organic layer was dried over anhydrous sodium sulphate, filtered and evaporated. The resulting residue was purified by silica gel chromatography (9:1 CH₂Cl₂:MeOH) to give 5 (1.2 g, 64%) as a foam. MS (ESI): m/z 537 (M+H).

### Example 6

### 10-{3-[4-(2-Amino)ethylpiperazinyl]propyl]}2-trifluoromethylphenothiazine (Compound 6)

Compound 5 (1.20 g, 2.23 mmol) was dissolved in dry CH₂Cl₂ (15 mL) and TFA (1.2 mL, 10.5 mmol) was added dropwise to this solution at 0° C. The solution was stirred at room temperature overnight. The reaction mixture was diluted with CH₂Cl₂ and pH adjusted to 8 by addition of saturated aqueous sodium bicarbonate. The layers were separated, and the aqueous layer was extracted with CH₂Cl₂ (2×20 mL). The combined organic layers were washed with saturated sodium chloride solution (10 mL), dried over anhydrous sodium sulphate and evaporated. The resulting residue 6 was taken on without any further purification. MS (ESI): m/z 437 (M+H).

### Example 7

### N-Boc protected ICI-735 (Compound 7)

To a solution of N-Boe glycine (0.48 g, 2.75 mmol), HATU (1.1 g, 2.89 mmol) and the phenothiazine piperazine 6 (1.0 g, 2.29 mmol) in CH₂Cl₂ (15 mL) was added DIPEA (1 mL) and the mixture was stirred at room temperature for 12 h. The reaction mixture was evaporated and the residue was purified by a silica gel column chromatography (9:1 CH₂Cl₂:MeOH) to give amide 7 (0.75 g, 55%) as a foam. MS (ESI): m/z 594 (M+H).

### Example 8

### ICI-735 (Compound 8)

Compound 7 (640 mg, 1.07 mmol) was dissolved in dry CH₂Cl₂ (10 mL) and TFA (0.6 mL, 5.26 mmol) was added dropwise to this solution at 0° C. The solution was stirred at room temperature overnight. The reaction mixture was evaporated and residue was purified by reverse phase HPLC on a C18 column (acetonitrile:water:TFA, gradient elution) to give the desired product 8 (620 mg 70%) as a white solid after lyophilization. MS (ESI): m/z 494 (M+H).

### Example 9

### 10-(3-Chloropropyl)-2-dimethylsulfamidophenothiazine (Compound 9)

To a stirred solution of 2-dimethylaminosulfonyl phenothiazine (3.06 g, 10 mmol) and sodium hydride (0.6 g, 12 mmol) in dry toluene (35 mL) was added 1-bromo-3-chloropropane (1.8 g, 1.15 mmol). The reaction mixture was stirred for 12 h at 110° C. under an atmosphere of argon. The solution was cooled to room temperature and poured into an ice-water mixture, the crude product was extracted with ethyl acetate (2×25 mL) and the organic phase was dried over anhydrous sodium sulphate. Final purification was performed by column chromatography (7:3 hexane:ethyl acetate) on silica gel to give 9 (2.5 g, 65%) as an oil.

### Example 10

### 10-{3-[4-(N-Boc-2-amino)ethylpiperazinyl]propyl}-2-dimethylsulfamidolphenothiazine (Compound 10)

To a stirred solution of the phenothiazine chloro derivative 9 (382 mg, 1.0 mmol), potassium carbonate (500 mg, 3.62 mmol), and 1-(2-N-Boc-aminoethyl)piperazine (229 mg, 1.0 mmol) in methyl ethyl ketone (20 mL) was added sodium iodide (150 mg, 1 mmol). The reaction mixture was stirred for 2411 at reflux under an atmosphere of argon. The reaction mixture was filtered, and the filtrate was concentrated under vacuum. The residue was partitioned between ethyl acetate (20 mL) and brine (10 mL). The organic layer was dried over anhydrous sodium sulphate, filtered, and evaporated. The resulting residue was purified by silica gel chromatography (9:1 CH₂Cl₂:MeOH) to give Compound 10 (410 mg, 71%) as a foam. MS (ESI): m/z 576 (M+H),

### Example 11

### ICI-715 (Compound 11)

Compound 10 (410 mg, 0.71 mmol) was dissolved in dry CH₂Cl₂ (5 mL) and TFA (0.4 mL, 3.5 mmol) was added dropwise to this solution at 0° C. The solution was stirred at room temperature overnight. The reaction mixture was evaporated and residue was purified by reversed phase HPLC on a C18 column (acetonitrile:water:TFA, gradient elution) to give the desired product 11 (325 mg, 56%) as a white solid after lyophilization. MS (ESI): m/z 476 (M+H).

### Example 12

### N-Boc-4-(3-bromopropyl)piperidine (Compound 12)

N-Boc-4-(3-hydroxypropyl)piperidine (160 mg, 0.658 mmol) was dissolved in dry THF (5 mL), and carbon tetrabromide (265 mg, 0.79 mmol) was added. Then a solution of triphenylphosphine (207 mg, 0.79 mmol) in dry tetrahydrofuran (2 mL) was added dropwise over 2 h. The mixture was stirred at room temperature for 18 h, and then diluted with diethyl ether (5 mL). The reaction mixture was filtered, the filtrate concentrated under vacuum, and the resulting residue was purified by silica gel column chromatography (9:1 hexane:ethyl acetate) to give compound 12 (143 mg, 72%) as an oil.

### Example 13

### 10-[3-(N-Boc-4-piperidyl)propyl]-2trifluoromethylphenothiazine (Compound 13)

To a stirred solution of 2-trifluoromethylphenothizine 1 (400 mg, 1.5 mmol), sodium hydride (100 mg, 2 mmol) in DME (10 mL) at 90° C. was added N-Boc-4-(3-bromopropyl)piperidine (Compound 12, 380 mg, 1.24 mmol) dropwise under an atmosphere of argon. The reaction mixture was stirred for 12 h at reflux. The reaction mixture was filtered and the filtrate was concentrated under vacuum. The residue was partitioned between ethyl acetate (25 mL) and brine (10 mL). The organic layer was dried over anhydrous sodium sulphate, filtered, and evaporated. The resulting residue was purified by silica gel column chromatography (8:2 hexane:ethyl acetate) on silica gel to give phenothiazine derivative 13 (425 mg, 70%) as a solid. MS (ESI): m/z 493 (M+H).

### Example 14

### ICI-824 (Compound 14)

Compound 13 (200 mg, 0.4 mmol) was dissolved in dry CH₂Cl₂ (5 mL) and TFA (0.2 mL, 1.75 mmol) was added dropwise to this solution at 0° C. The solution was stirred at room temperature overnight. The reaction mixture was evaporated and residue was purified by reversed phase HPLC on a C18 column (acetonitrile:water:TFA, gradient elution) to give the desired product 14 (125 mg, 61%) as a white solid after lyophilization. MS (ESI): m/z 393 (M+H).

### Example 15

### 10-{3-[1-(N-boc-2-amino)ethyl-4-piperidyl]propyl}-2-trifluoromethylphenothiazine (Compound 15)

To a solution of piperidine derivative 14 (160 mg, 0.4 mmol) and potassium carbonate (500 mg, 3.62 mmol) in dry DMF (5 mL) was added N-Boc-2-aminoethylbromide (137 mg, 0.6 mmol), and the solution was stirred for 24 h at room temperature. The mixture was diluted with ethyl ether (20 mL), washed with water (2×10 mL), and brine (5 mL), dried over anhydrous sodium sulphate, and then concentrated under vacuum. The residue was purified by silica gel column chromatography (9:1 CH₂Cl₂:MeOH) to give compound 15 (152 mg, 70%) as an oil. MS (ESI): m/z 536 (M+H).

### Example 16

### 10-{3-[1-(N-Boc-3-amino)propyl-4-piperidyl]propyl}-2-trifluoromethylphenothiazine (Compound 16)

To a solution of piperidine derivative 14 (526 mg, 1.34 mmol) and potassium carbonate (1.0 g, 7.25 mmol) in dry DMF (5 mL) was added N-Boc-3-aminopropylbromide (627 mg, 2.63 mmol) and the solution was stirred for 24 h at room temperature. The mixture was diluted with diethyl ether (10 mL), washed with water (2×10 mL), and brine (5 mL), dried over anhydrous sodium sulphate, and then concentrated under vacuum. The residue was purified by silica gel column chromatography (9:1 CH₂Cl₂:MeOH) to give compound 16 (325 mg, 44%) as an oil, MS (ESI): m/z 550 (M+H).

### Example 17

### ICI-847 (Compound 17)

Compound 16 (120 mg, 0.22 mmol) was dissolved in dry CH₂Cl₂ (5 mL) and TFA (0.2 mL, 1.75 mmol) was added dropwise to this solution at 0° C. The solution was stirred at room temperature overnight. The reaction mixture was evaporated and residue was purified by reversed phase HPLC on a CIS column (acetonitrile:water:TFA, gradient elution) to give the desired product 17 (52 mg, 35%) as a white solid after lyophilization. MS (ESI): m/z 450 (M+H).

### Example 18

### 10-{3-[1-(2-Amino)ethyl-4-piperidyl]propyl}-2-trifluoromethyl-phenothiazine (Compound 18)

Compound 15 (600 mg, 1.12 mmol) was dissolved in dry CH₂Cl₂ (10 mL) and TFA (0.75 mL, 6.57 mmol) was added dropwise to this solution at 0 C. The solution was stirred at room temperature overnight. The reaction mixture was diluted with CH₂Cl₂ and pH adjusted to 8 by addition of saturated aqueous sodium bicarbonate. The layers were separated, and aqueous layer was extracted with CH₂Cl₂ (2×20 mL). The combined organic layers were washed with saturated sodium chloride solution (10 mL), dried over anhydrous sodium sulphate and evaporated, The resulting amine 18, was taken on without any further purification. MS (ESI): m/z 436 (M+H).

### Example 19

### N-Boc protected ICI-849 (Compound 19)

To a solution of N-Boc sarcosine (286 mg, 1.51 mmol), HATU (574 mg, 1.51 mmol) and the propyl ethylpiperidine amine 18 (550 mg, 1.26 mmol) in CH₂Cl₂ (15 mL) was added DIPEA (0.5 mL) and the mixture was stirred at room temperature for 12 h. The reaction mixture was evaporated and residue was purified by a silica gel column chromatography (9:1 CH₂Cl₂:MeOH) to give amide 19 (400 mg, 52%) as a foam.

MS (ESI): nth 607 (M+H).

### Example 20

### ICI-849 (Compound 20)

Compound 19 (200 mg, 0.33 mmol) was dissolved in dry CH₂Cl₂ (5 mL) and TFA (0.2 mL, 1.75 mmol) was added dropwise to this solution at 0 C. The solution was stirred at room temperature overnight. The reaction mixture was evaporated and residue was purified by reversed phase HPLC on a C18 column (acetonitrile:water:TFA, gradient elution) to give the desired product 20 (110 mg, 45%) as a white solid after lyophilization. MS (ESI): m/z 507 (M+H).

### Example 21

### 10-(4-chlorobutyl)-2-trifluoromethylphenothiazine (Compound 21)

To a stirred solution of 2-trifluoromethylphenothiazine 1 (4.0 g, 15 mmol), sodium hydride (1.2 g, 24 mmol) in dry toluene (40 mL), 1-bromo 4-chlorobutane (3.0 g, 17.6 mmol) was added. The reaction mixture was stirred for 18 hours at 110° C. under an atmosphere of argon. The solution was cooled to room temperature and poured into an ice-water mixture. The crude product was extracted with ethyl acetate (3×50 mL) and the organic phase was dried over sodium sulphate. Final purification was performed by column chromatography (9:1 hexane:ethyl acetate) on silica gel to give Compound 21 (3.5 g, 65%) as an oil.

### Example 22

### 10-{4-[4-(N-Boc-2-amino)ethylpiperazinyl]butyl}-2-trifluoromethyl-phenothiazine (Compound 22)

To a stirred suspension of the chlorobutyl derivative 21 (3.57 g, 10 mmol), potassium carbonate (4.0 g, 28.98 mmol), 1-(2-N-Boc-aminoethyl)piperazine (2.6 g, 11.35 mmol) in methyl ethyl ketone (40 mL) was added sodium Iodide 2.5 g, 16 mmol). The reaction mixture was stirred for 24 h at reflux under an atmosphere of argon, The reaction mixture was filtered, and the filtrate was concentrated under vacuum. The residue was partitioned between ethyl acetate (50 mL) and brine (25 mL). The organic layer was dried over anhydrous sodium sulphate, filtered and evaporated. The resulting residue was purified by silica gel chromatography (9:1 CH₂Cl₂:MeOH) to give Compound 22 (4.0 g, 72%) as a foam. MS (ESI): m/z 551 (Mill).

### Example 23

### ICI-953 (Compound 23)

Compound 22 (152 mg, 0.28 mmol) was dissolved in dry CH₂Cl₂ (5 mL) and TFA (0.2 mL, 1.75 mmol) was added dropwise to this solution at 0° C. The solution was stirred at room temperature overnight. The reaction mixture was evaporated and residue was purified by reversed phase HPLC on a C18 column (acetonitrile:water:TFA, gradient elution) to give the desired product 23 (150 mg, 68%) as a white solid after lyophilization. MS (ESI): m/z 451 (M+H).

### Example 24

### N-Boc protected ICI-954 (Compound 24)

To a solution of N-Boc-glycine (0.7 g, 4.0 mmol), HATU (1.6 g, 4.2 mmol) and amine 23 (1.5 g, 3.3 mmol) in CH₂Cl₂ (20 mL) was added DIPEA (1.5 mL) and the mixture was stirred at room temperature for 12 h. The reaction mixture was evaporated and residue was purified by a silica gel column chromatography (9:1 CH₂Cl₂:MeOH) to give amide 24 (1.2 g, 60%) as a foam.

### Example 25

### ICI-954 (Compound 25)

Compound 24 (250 mg, 0.41 mmol) was dissolved in dry CH₂Cl₂ (5 mL) and TFA (0.2 mL, 1.75 mmol) was added dropwise to this solution at 0° C. The solution was stirred at room temperature overnight. The reaction mixture was evaporated and residue was purified by reversed phase HPLC on a C18 column (acetonitrile:water:TFA, gradient elution) to give the desired product 25 (210 mg, 60%) as a white solid after lyophilization. MS (ESI): m/z 508 (M+H).

### Example 26

### N-Boc-4-(2-bromoethyl)piperidine (Compound 26)

N-Boc-4-(2-hydroxyethyl)piperidine (0 95 g, 4.17 mmol) was dissolved in dry THF (20 mL), and carbon tetrabromide (1.34 g, 4.0 mmol) was added. Then a solution of triphenylphosphine (1.15 g, 4.38 mmol) in dry tetrahydrofuran (2 mL) was added dropwise over 2 h. The mixture was stirred at room temperature for 18 h, and then diethyl ether (50 mL) added to the mixture. The reaction mixture was filtered, and filtrate concentrated under vacuum. The resulting residue was purified by silica gel column chromatography (9:1 hexane:ethyl acetate) to give Compound 26 (1.05 g, 86%) as an oil.

### Example 27

### 10-[2-(N-Boc-4-piperidyl)ethyl]-2-trifluoromethylphenothiazine (Compound 27)

To a stirred solution of 2-trifluoromethylphenothizine 1 (0.91 g, 3.42 mmol), sodium hydride (0.2 g, 4.0 mmol) in DME (20 mL) at 90° C. was added N-Boc-4-(2-bromoethyl)piperidine 26 (1.0 g, 3.42 mmol) dropwise under an atmosphere of argon. The reaction mixture was stirred for 12 h at reflux temperature. The reaction mixture was filtered, and filtrate was concentrated under vacuum. The residue was partitioned between ethyl acetate (25 mL) and brine (10 mL). The organic layer was dried over anhydrous sodium sulphate, filtered, and evaporated. The resulting residue was purified by column chromatography (8:2 n-hexane:ethyl acetate) on silica gel to give phenothiazine derivative 27 (0.3 g, 18%) as a foam. MS (ESI): m/z 479 (M+H).

### Example 28

### ICI-1007 (Compound 28)

Compound 27 (70 mg, 0.15 mmol) was dissolved in dry CH₂Cl₂ (5 mL) and TFA (0.1 mL, 0.88 mmol) was added dropwise to this solution at 0° C. The solution was stirred at room temperature overnight. The reaction mixture was evaporated and residue was purified by reversed phase HPLC on a C18 column(acetonitrile:water:TFA, gradient elution) to give the desired product 28 (50 mg, 69%) as a white solid after lyophilization, MS (ESI): m/z 379 (M+H).

### Example 29

### 10-{2-[1-(N-Boc-2-amino)ethyl-4-piperidyl]ethyl}-2-trifluoromethylphenothiazine (Compound 29)

To a solution of the piperidine derivative 28 (145 mg, 0.38 mmol) and potassium carbonate (500 mg, 3.62 mmol) in dry DMF (5 mL) was added N-Boc-2-aminoethylbromide (102 mg, 0.45 mmol), and the solution was stirred for 24 h at room temperature. The mixture was diluted with diethyl ether (20 mL), washed with a water (2×10 mL), brine (5 mL), dried over anhydrous sodium sulphate, and then concentrated under vacuum. The residue was purified by silica gel column chromatography (9:1 CH₂Cl₂:MeOH) to give Compound 29 (145 mg, 73%) as an oil. MS (ESI): m/z 522 (M+H).

### Example 30

### ICI-1008 (Compound 30)

Compound 29 (100 mg, 0.19 mmol) was dissolved in dry CH₂Cl₂ (5 mL) and TFA (0.2 mL, 1.75 mmol) was added dropwise to this solution at 0° C. The solution was stirred at room temperature overnight. The reaction mixture was evaporated and residue was purified by reversed phase HPLC on a C18 column (acetonitrile:water:TFA, gradient elution) to give the desired product 30 (52 mg, 42%) as a white solid after lyophilization. MS (ESI): m/z 422 (M+H).

### Example 31

### Biological Assays

### Serotonin-Receptor Binding Assays

The methods employed in this study have been adapted from the scientific literature to maximize reliability and reproducibility. Reference standards were run as an integral part of each assay to ensure the validity of the results obtained. Assays were performed under conditions as described below. Where presented, IC₅₀ values were determined by a non-linear, least squares regression analysis using the Data Analysis Toolbox (MDL Information Systems, San Leandro, Calif., USA). Where inhibition constants (Kᵢ) are presented, the Kᵢ values were calculated using the equation of Cheng and Prusoff (Cheng, Y., Prusoff, W. H., Biochem. Pharmacol. 22:3099-3108, 1973) using the observed IC₅₀ of the tested compound, the concentration of radioligand employed in the assay, and historical values for the K_{D} of the ligand. Where presented, the Hill coefficient (nᵤ), defining the slope of the competitive binding curve, was calculated using the Data Analysis Toolbox. Hill coefficients that differ by more than 10 may suggest that the binding displacement does not follow the laws of mass action with a single binding site.

Tables 4-7 illustrate the results for the biochemical assays set forth in Tables 2 and 3. The experiments measure the ability of compounds of the invention to displace known ligands from serotonin receptors. The data for 5-HTR-1A and 5-HTR-1B, set forth in Tables 4-7, demonstrate effectiveness of compounds of the invention in specific displacement of ligands.

TABLE 2
Experimental data for experiments 271000 (Table 4) and 271110 (Table 5)

| 271000 Serotonin (5-Hydroxytryptamine) | | 271000 Serotonin (5-Hydroxytryptamine) | |
|---|---|---|---|
| 5-HT₁, Non-Selective | | 5-HT_{1A} | |
| Source: | Wistar Rat cerebral cortex | Source: | Human recombinant CHO cells |
| Ligand: | 2 nM [³H] Serotonin (5-HT) | Ligand | 1.5 nM [³H] 8-OH-DPAT |
| Vehicle: | 1% DMSO | Vehicle: | 1% DMSO |
| Incubation | 10 minutes @ 37° C. | Incubation | 60 minutes @ 25° C. |
| Time/Temp: | | Time/Temp: | |
| Incubation | 50 mM Tris-HCl, pH 7.4, | Incubation | 50 mM Tris-HCl, pH 7.4, 0.1% |
| Buffer: | 0.1% Ascorbic Acid, 10 μM | Buffer: | Ascorbic Acid, 0.5 mM EDTA, |
| | Pargyline, 4 mM CaCl₂ | | 10 mM MgSO₄ |
| Non-Specific | 10 μM Serotonin (5-HT) | Non-Specific | 10 μM Metergoline |
| Ligand: | | Ligand: | |
| K_{D:} | 0.61 nM * | K_{D:} | 2 nM * |
| B_{MAX:} | 0.58 pmole/mg Protein * | B_{MAX:} | 1.3 pmole/mg Protein * |
| Specific | 80% * | Specific | 75%* |
| Binding: | | Binding: | |
| Quantitation | Radioligand Binding | Quantitation | Radioligand Binding |
| Method: | | Method: | |
| Significance | ≧50% of max stimulation or | Significance | ≧50% of max stimulation or |
| Criteria: | inhibition | Criteria: | inhibition |

TABLE 3
Experimental data for experiments 271200 (Table 6) and 271600 (Table 7).

| 271000 Serotonin (5-Hydroxytryptamine) | | 271000 Serotonin (5-Hydroxytryptamine) | |
|---|---|---|---|
| 5-HT_{1B} | | 5-HT₂, Non-Selective | |
| Source: | Wistar Rat cerebral cortex | Source: | Wistar Rat brain |
| Ligand: | 10 pM [¹²⁵I] Cyanopindolol | Ligand | 0.5 nM [³H] Ketanserin |
| Vehicle: | 1% DMSO | Vehicle: | 1% DMSO |
| Incubation | 90 minutes @ 37° C. | Incubation | 40 minutes @ 25° C. |
| Time/Temp: | | Time/Temp: | |
| Incubation | 50 mM Tris-HCl, pH 7.4, 154 | Incubation | 50 mM Tris-HCl, pH 7.4 |
| Buffer: | mM NaCl, 10 μM Pargyline, | Buffer: | |
| | 30 μM Isoprenaline | | |
| Non-Specific | 10 μM Serotonin (5-HT) | Non-Specific | 1 μM Ketanserin |
| Ligand: | | Ligand: | |
| K_{D:} | 0.19 nM * | K_{D:} | 0.82 nM* |
| B_{MAX:} | 0.14 pmole/mg Protein * | B_{MAX:} | 0.52 pmole/mg Protein * |
| Specific | 70% * | Specific | 92% * |
| Binding: | | Binding: | |
| Quantitation | Radioligand Binding | Quantitation | Radioligand Binding |
| Method: | | Method: | |
| Significance | ≧50% of max stimulation or | Significance | ≧50% of max stimulation or |
| Criteria: | inhibition | Criteria: | inhibition |

TABLE 4
Assay 271000 - Serotonin (5-Hydroxytryptamine)
5-HT₁, Non Selectiv

| | | |
|---|---|---|
| COMPOUND CODE | CONC. | % INHIBITION |

| | | | |
|---|---|---|---|
| ICI-685 | 10 | μM | 32 |
| | 0.1 | μM | −3 |
| ICI-715 | 10 | μM | 62 |
| | 0.1 | μM | 15 |
| ICI-735 | 10 | μM | 63 |
| | 0.1 | μM | 16 |
| ICI-824 | 10 | μM | 11 |
| | 0.1 | μM | −14 |
| ICI-847 | 10 | μM | −7 |
| | 0.1 | μM | −11 |
| ICI-849 | 10 | μM | 10 |
| | 0.1 | μM | −2 |
| ICI-953 | 10 | μM | −1 |
| | 0.1 | μM | −10 |
| ICI-954 | 10 | μM | 18 |
| | 0.1 | μM | −3 |
| ICI-1007 | 10 | μM | 24 |
| | 0.1 | μM | −12 |
| ICI-1008 | 10 | μM | 3 |
| | 0.1 | μM | −20 |
| ICI-1175 | 10 | μM | 69 |
| | 0.1 | μM | 18 |
| ICI-1176 | 10 | μM | 75 |
| | 0.1 | μM | 32 |

TABLE 5
Assay 271000 - Serotonin (5-Hydroxytryptamine) 5-HT_{1A}

| | | |
|---|---|---|
| COMPOUND CODE | CONC. | % INHIBITION |

| | | | |
|---|---|---|---|
| ICI-685 | 10 | μM | 26 |
| | 0.1 | μM | 1 |
| ICI-715 | 10 | μM | 87 |
| | 0.1 | μM | 5 |
| ICI-735 | 10 | μM | 89 |
| | 0.1 | μM | 14 |
| ICI-824 | 10 | μM | 53 |
| | 0.1 | μM | 2 |
| ICI-847 | 10 | μM | 74 |
| | 0.1 | μM | −8 |
| ICI-849 | 10 | μM | 65 |
| | 0.1 | μM | 18 |
| ICI-953 | 10 | μM | 54 |
| | 0.1 | μM | 5 |
| ICI-954 | 10 | μM | 56 |
| | 0.1 | μM | 6 |
| ICI-1007 | 10 | μM | 48 |
| | 0.1 | μM | 2 |
| ICI-1008 | 10 | μM | 69 |
| | 0.1 | μM | 4 |
| ICI-1175 | 10 | μM | 89 |
| | 0.1 | μM | 21 |
| ICI-1176 | 10 | μM | 93 |
| | 0.1 | μM | 32 |

TABLE 6
Assay 271200 - Serotonin (5-Hydroxytryptamine) 5-HT_{1B}

| | | |
|---|---|---|
| COMPOUND CODE | CONC. | % INHIBITION |

| | | | |
|---|---|---|---|
| ICI-685 | 10 | μM | 10 |
| | 0.1 | μM | 6 |
| ICI-715 | 10 | μM | 90 |
| | 0.1 | μM | 23 |
| ICI-735 | 10 | μM | 86 |
| | 0.1 | μM | 13 |
| ICI-824 | 10 | μM | 6 |
| | 0.1 | μM | 2 |
| ICI-847 | 10 | μM | −33 |
| | 0.1 | μM | −5 |
| ICI-849 | 10 | μM | −2 |
| | 0.1 | μM | −7 |
| ICI-953 | 10 | μM | 2 |
| | 0.1 | μM | 7 |
| ICI-954 | 10 | μM | −1 |
| | 0.1 | μM | 4 |
| ICI-1007 | 10 | μM | 48 |
| | 0.1 | μM | 8 |
| ICI-1008 | 10 | μM | 0 |
| | 0.1 | μM | −3 |
| ICI-1175 | 10 | μM | 29 |
| | 0.1 | μM | 1 |
| ICI-1176 | 10 | μM | 103 |
| | 0.1 | μM | 92 |

TABLE 7
Assay 271600 - Serotonin (5-Hydroxytryptamine)
5-HT₂, Non-Selective

| | | |
|---|---|---|
| COMPOUND CODE | CONC. | % INHIBITION |

| | | | |
|---|---|---|---|
| ICI-685 | 10 | μM | 82 |
| | 0.1 | μM | 31 |
| ICI-715 | 10 | μM | 83 |
| | 0.1 | μM | 18 |
| ICI-735 | 10 | μM | 91 |
| | 0.1 | μM | 62 |
| ICI-824 | 10 | μM | 84 |
| | 0.1 | μM | 28 |
| ICI-847 | 10 | μM | 82 |
| | 0.1 | μM | 3 |
| ICI-849 | 10 | μM | 85 |
| | 0.1 | μM | 15 |
| ICI-953 | 10 | μM | 71 |
| | 0.1 | μM | 16 |
| ICI-954 | 10 | μM | 89 |
| | 0.1 | μM | 21 |
| ICI-1007 | 10 | μM | 87 |
| | 0.1 | μM | 51 |
| ICI-1008 | 10 | μM | 94 |
| | 0.1 | μM | 38 |
| ICI-1175 | 10 | μM | 82 |
| | 0.1 | μM | 67 |
| ICI-1176 | 10 | μM | 79 |
| | 0.1 | μM | 19 |

### Example 32

### Pharmacological Evaluation of Compounds ICI-685 and ICI-735 in Model of LPS-Mediated Cytokine Production

Bolus injection of lethal or sub-lethal doses of lipopolysaccharide (LPS; the major component of bacterial cell walls) results in a rapid and transient rise in serum cytokine levels (e.g. TNF-α) in mammals. This animal model was originally developed to mirror certain aspects of septic shock in humans; however, there is poor correlation between efficacy in LPS-rodent models and clinical efficacy. However, this model may be an effective first-line general inflammation model and could be useful in determining the anti-inflammatory potential of test compounds. A variety of clinically approved anti-inflammatory compounds, including glucocorticoids, NSAIDS and COX-2 inhibitors are extremely effective in this model. Compounds ICI-685 and ICI-735 were tested for their ability to inhibit LPS-stimulated TNF-α and IL-1β production.

Both ICI-685 and ICI-735 were formulated in water. For the time course study, both drugs were formulated at a concentration of 1 mg/ml and dosed 10 ml/kg to produce a dose of 10 mg/kg. For the dose-response study, drug was formulated at concentrations of 0.05, 0.2 and 0.5 mg/ml and dosed at a volume of 10 ml/kg to produce doses of 0.5, 2 and 5 mg/kg, respectively. Animals were dosed IV or IP.

CD1:ICR mice were obtained from Harlan (Indianapolis, Ind.) at 6 weeks of age. Animals were housed 5 per cage, kept on a 12 hr light dark cycle and fed food and water ad libitum. Animals were tested at 8-10 weeks of age.

Lipopolysaccharide (heat killed E. coli 0127:B5; Sigma Aldrich) was prepared in distilled water at a concentration of 0.025 mg/ml. LPS was dosed at a volume of 10 ml/kg (IP) to produce a final dose of 0.25 mg/kg (approximately 7.5 μg/mouse). Drugs were dosed prior to LPS administration as indicated elsewhere herein. Blood was collected by retro-orbital eye bleed 90 minutes after LPS administration, Serum was prepared from blood and TNF-α and IL-1β levels were measured by using the OPT-EIA mouse TNF-α and IL-1β ELISA kits (BD Biosciences) as per directions of the manufacturer.

The first study was designed to determine the optimal route of administration and the optimal pre-treatment time. Two pre-dose time course studies were conducted. The first (Study 1A) was conducted with 3 pre-dose time points (2, 6 and 18 hrs). The second was conducted at 0, 1 and 2 hr predose time points. For both studies, drug was dosed IP or IV.

Using data from the first study, a second study was conducted that was designed to measure dose-response activity of each compound. Compounds were tested at doses of 0., 2.0 and 5 mg/kg using the route and pre-treatment time that produced the best activity.

LPS-stimulated increases in both TNFα and IL-1β to levels consistent with those of previous studies. Consistent with these previous studies, TNFα was much more responsive than IL-1β to these LPS-stimulated increases. Serum TNFα levels were increased from undetectable levels to between 3 and 8 ng/ml. IL-1β levels were elevated from baseline levels of between 50 and 100 pg/ml to an LPS-stimulated level of 200 to 350 pg/ml.

Both ICI-685 and ICI-735 inhibited LPS-stimulated TNFα secretion. For both compounds, the optimal pre-dose time period for TNFα inhibition was between 0 and 2 hrs with IV administration producing a slightly better inhibition than IP administration. For the subsequent dose-response study, animals were dosed with a pre-dose period of 1 hr via IV administration. The test drugs used in these studies did not inhibit LPS-mediated increases in IL-1β levels in a reproducible fashion. These data are consistent with our previous studies that demonstrate that IL-1β is less responsive than TNFα to the inhibitory activity of these class of molecules.

For the dose-response study, both compounds inhibited at concentrations of 5 mg/kg, but not at lower doses. In combination with the pre-dose time course (which were dosed at 10 mg/kg), it appears that the most active dose levels for both compounds are 10 mg/kg.

For the current studies, there appears to be a discrepancy between the two time courses. Specifically, in the first time course ICI-685 did not inhibit TNFα levels at the 2 hr pretreatment period (IV administration). However, in the second pre-dose time course study, ICI-685 inhibited TNFα by 70%. As will be understood by the skilled artisan, effective dose-ranges in this type of LPS study for any compound can fluctuate from 5 to 10 fold. Immune function and cytokine responsiveness can be altered by (for example) environmental conditions (previous and current), age of animals, feeding state, time of study and LPS preparation.

### Example 33

### Pharmacological Evaluation of Compounds ICI-715, ICI-824, ICI-953 and ICI-954 in Model of LPS-Mediated Cytokine Production

Compounds ICI-715, ICI-824, ICI-953 and ICI-954 were formulated in water. For the time course study, drugs were formulated at a concentration of 1 mg/ml and dosed 10 ml/kg to produce a dose of 10 mg/kg. For the dose-response study, drugs were formulated at concentrations of 0.05, 0.2 and 0.5 mg/ml and dosed at a volume of 10 ml/kg to produce doses of 0.5, 2 and 5 mg/kg, respectively. Animals were dosed IV or IP.

CD1:ICR mice were obtained from Harlan (Indianapolis, Ind.) at 6 weeks of age. Animals were housed 5 per cage, kept on a 12 hr light dark cycle and fed food and water ad libitum. Animals were tested at 8-10 weeks of age.

Lipopolysaccharide (heat killed E. coli 0127:B5; Sigma Aldrich) was prepared in distilled water at a concentration of 0.025 mg/ml, LPS was dosed at a volume of 10 ml/kg (IP) to produce a final dose of 0.25 mg/kg (approximately 7.5 pg/mouse). Drugs were dosed prior to LPS administration as indicated above. Blood was collected by retro-orbital eye bleed 90 minutes after LPS administration. Serum was prepared from blood and TNF-α and IL-1β levels were measured by using the OPT-EIA mouse TNF-α and IL-1β ELISA kits (BD Biosciences) as per directions of the manufacturer.

The first study was designed to determine the optimal route of administration and the optimal pre-treatment time. One pre-dose time course was conducted (for all compounds) with compounds administered at 0, 1 and 2 hrs prior to LPS treatment. For both studies, drug was dosed IP or IV. The IP study and IV study were conducted on separate days.

Using data from the first study, a second study was conducted that was designed to measure dose-response activity of each compound. Compounds were tested at doses of 0.5, 2.0 and 5 mg/kg using the route and pre-treatment time that produced the best activity.

In the present studies, LPS-stimulated increases in both TNFα and IL-1β to levels consistent with those of previous studies. Consistent with these previous studies, TNFα was much more responsive than IL-1β to these LPS-stimulated increases. Serum TNFα levels were increased from undetectable levels to between 1 and 7 ng/ml. IL-1β levels were elevated from baseline levels of between 50 and 100 pg/ml to an LPS-stimulated level of 200 to 350 pg/ml.

All four compounds inhibited LPS-stimulated TNFα secretion. The optimal pre-dose time period for TNFα inhibition was 1 hr. IP administration produced slightly better inhibition than IV administration for ICI-824, ICI-953 and ICI-954. IV administration of ICI-715 produced slightly better inhibition than IV administration. This predose time period and these routes were selected for the subsequent dose-response analysis.

For the dose-response study, the dose-range was between 0.5 and 5 mg/kg. Administration of ICI-715 (IV) produced at least a 50% inhibition of TNFα at all doses tested. ICI-824, ICI-953 and ICI-954 (IP) were ineffective up to a dose of 5 mg/kg. In combination with the pre-dose time course (which were dosed at 10 mg/kg), it appears that the most active dose levels for these last three compounds are 10 mg/kg. It also appears that ICI-715 may be more potent than these other compounds. However, ICI-715 was dosed IV, and the other compounds were dosed IP.

### Example 34

### Synthesis of Glycinamides

### 10-(3-Chloropropyl)-2-trifluoromethylphenothiazine (Compound 32)

To a stirred suspension of 2-(trifluoromethyl)phenothiazine (Compound 31) (2.00 g, 7.49 mmol) and NaH(0.5 g, 10.42 mmol) in dry toluene (30 mL) was added 1-bromo-3-chloropropane (1.57 g, 10 mmol). The reaction mixture was stirred for 18 hours at 110° C. under an atmosphere of argon. The solution was cooled to room temperature and poured into an ice-water mixture, the crude product was extracted with ethyl acetate (3×50 mL) and the organic phase was dried over Na₂SO₄. Final purification was performed by column chromatography (9:1 n-hexane:ethyl acetate) after absorbing the crude product on silica gel to give Compound 32 (1.5 g, 58%) as a solid.

### 10-{3-[4-(N-Boc-2-amino)ethylpiperazinyl]propyl}-2-trifluoromethylphenothiazine (Compound 33)

To a stirred suspension of chloropropyl derivative (Compound 32) (1.2 g, 3.5 mmol), K₂CO₃ (1.5 g, 10.86 mmoles), and 1-(2-N-boc-aminoethyl)piperazine (0.78 g, 3.5 mmol) in methyl ethyl ketone (30 mL) was added NaI (0.9 g, 6 mmol). The reaction mixture was stirred for 24 h at reflux temperature under atmosphere of argon. The reaction mixture was filtered, and the filtrate was concentrated under vacuum. The residue was partitioned between ethyl acetate (30 mL) and brine (15 mL). The organic layer was dried over anhydrous Na₂SO₄, filtered and evaporated. The resulting residue was purified by silica gel chromatography (9:1 dichloromethane:MeOH) to give (Compound 33) (1.2 g, 64%) as a foam. MS (ESI) 537 (MH).

### 10-{3-[4-(2-Amino)ethylpiperazinyl]propyl}2-trifluoromethylphenothiazine (Compound 34)

Compound 33 (1.20 g, 2.23 mmol) was dissolved in 15 mL of dry dichloromethane and TFA (1.2 mL, 10.5 mmol) was added dropwise to this solution at 0° C. The solution was stirred at room temperature overnight. The reaction mixture was diluted with dichloromethane and the pH adjusted to 8 by addition of saturated aqueous NaHCO₃. The layers were separated, and aqueous layer was extracted with dichloromethane (2×20 mL). The combined organic layers were washed with saturated NaCl solution (10 mL), dried over anhydrous Na₂SO₄ and evaporated. The resulting residue (Compound 34) was taken on without further purification. MS (ESI) 437 (MH).

### tert-Butyl (2-methyl-1-oxo-1-((2-(4-(3-(2-(trifluoromethyl)-10H-phenothiazin-10-yl)propyl)piperazin-1-yl)ethyl)amino)propan-2-yl)carbamate, (Compound 35a)

To a solution of Boc-aminoisobutyric acid (0.127 g, 0.62 mmol), HATU (0.24 g, 0.62 mmol) and Compound 34 (0.23 g, 0.52 mmol) in dichloromethane (15 mL) was added DIPEA (0.4 mL) and the mixture was stirred at room temperature for 12 h. The reaction mixture was evaporated and the residue was purified by silica gel column chromatography (9:1 dichloromethane:MeOH) to give amide (Compound 35a) (0.136 g, 55%) as a foam. MS (ESI) 622 (MH).

### 2-Amino-2-methyl-N-(2-(4-(3-(2-(trifluoromethyl)-10H-phenothiazin-10-yl)propyl)piperazin-1-yl)ethyl)propanamide (Compound 36a)

Compound 35a (R⁶═R⁷═CH₃, R⁸═NHBoc; 0136 g, 0.22 mmol) was dissolved in 10 mL of dry dichloromethane and TFA (0.6 mL, 5.26 mmol) was added dropwise to this solution at 0° C. The solution was stirred at room temperature overnight. The reaction mixture was evaporated and the residue was purified by reversed-phase HPLC to give the desired product, Compound 36a (R⁶═R⁷═CH₃, R⁸═NH₂; 132 mg, 70%) as a white solid after lyophilization, MS (LC/MS, EST): 522 (M+H). NMR (300 MHz, CDCl₃, δ): 8.7 (brs, 1H); 8.4 (s, 2H); 7.0-7.4 (m, 7H), 4.1 (t, 2H), 3.0-3.9 (m, 18H), 1.4 (s, 6H).

### Example 35

### General N-Formylation Step of Terminal Amino Group of Glycinamides

To a solution of Compound 36 (1 mmol) in DMF (15 mL) was added DIPEA (1 mmol) and p-nitrophenylformate (1.1 mmol) at room temperature. After stirring overnight, the reaction mixture was cooled, washed with 1N HCl, then water. The organic layer was separated, dried, and evaporated. The residue was purified by silica gel column chromatography to give Compound 37a (R⁵ is H).

This procedure may be used to prepare Compound 37b (2-formamido-N-(2-(4-(3-(2-(trifluoromethyl)-10H-phenothiazin-10-yl)propyl)piperazin-1-yl)ethyl)acetamide).

### Example 36

### General N-Acylation Step of Terminal Amino Group of Glycinamides

To a solution of Compound 36 (0.27 g, 0.52 mmol) in dichloromethane (15 mL) was added DIPEA (0.4 mL) and the mixture was cooled to 0° C. The reaction mixture was heated to reflux and a solution of the acyl chloride (0.57 mmol) in dichloromethane (15 mL) was added. After refluxing overnight, the reaction mixture was cooled, washed with 1N HCl, then water. The organic layer was separated, dried, and evaporated. The residue was purified by silica gel column chromatography to give N-acylated glycinamide (Compound 37).

### Example 37

### Alternative N-Acylation of Terminal Amino Group of Glycinamides

To a solution of R⁵COOH (0.62 mmol), HATU (0.24 g, 0.62 mmol) and Compound 36 (0.23 g, 0.52 mmol) in dichloromethane (15 mL) was added DIPEA (0.4 mL) and the mixture was stirred at room temperature for 12 h. The reaction mixture was evaporated and the residue was purified by silica gel column chromatography to give N-acylated glycinamide (Compound 37).

### Example 38

### General N-Sulfonylation Step of Terminal Amino Group of Glycinamides

To a solution of Compound 36 (0.27 g, 0.52 mop in dichloromethane (15 mL) was added DIPEA (0.4 mL) and the mixture was cooled to 0° C. The reaction mixture was heated to reflux and a solution of the sulfonyl chloride (0.57 mmol) in dichloromethane (15 mL) was added. After refluxing overnight, the reaction mixture was cooled, washed with 1N HCl, then water. The organic layer was separated, dried, and evaporated. The residue was purified by silica gel column chromatography to give N-sulfonyl glycinamides (Compound 38).

### Example 39

### General Synthesis of Amides

To a solution of the acid, such as 2,2-dimethylpropanoic acid (0.063 g, 0.62 mmol), HATU (0.24 g, 0.62 mmol) and Compound 34 (0.23 g, 0.52 mmol) in dichloromethane (15 mL) was added DIPEA (0.4 mL) and the mixture was stirred at room temperature for 12 h. The reaction mixture was evaporated and the residue was purified by silica gel column chromatography to give Compound 35b (N-(2-(4-(3-(2-(trifluoromethyl)-10H-phenothiazin-10-yl)propyl)piperazin-1-yl)ethyl)pivalamide; R⁶, R⁷, R⁸═CH₃).

### Example 40

### Biological Assay for Serotonin Binding Activity

The specific ligand binding to the serotonin 2A and 2B receptors (5-HT_{2A} and 5-HT_{2B}) was defined as the difference between the total binding and the nonspecific binding determined in the presence of an excess of unlabeled ligand. The serotonin 2A and 2B receptor binding were determined using both an agonist and an antagonist radioligand. The human cellular source for the serotonin binding assays, the specific radioligands and the assay conditions are listed in Table 8.

TABLE 8
Serotonin 5-HT_{2A} and 5-HT_{2B} Binding Assays

| | | | | | Non- | | | |
|---|---|---|---|---|---|---|---|---|
| Assay | Source | Ligand | Conc | K_{d} | specific | Incubation | Detection | Ref. |
| 5-HT_{2A} (h) | hr HEK- | [³H] | 0.5 nM | 0.6 nM | Ketanserin | 60 min, | Scintillation | 1 |
| antagonist | 293 cells | ketanserin | | | (1 μM) | RT | | |
| 5-HT_{2A} (h) | hr HEK- | [¹²⁵I] | 0.1 nM | 0.3 nM | (±)DOI | 60 min, | Scintillation | 2 |
| agonist | 293 cells | (±)DOI | | | (1 μM) | RT | | |
| 5-HT_{2B} (h) | hr CHO | [³H] | 2 nM | 2.4 nM | mesulergine | 60 min, | Scintillation | 3 |
| antagonist | cells | mesulergine | | | (10 μM) | RT | | |
| 5-HT_{2B} (h) | hr CHO | [¹²⁵I] | 0.2 nM | 0.2 nM | (±)DOI | 60 min, | Scintillation | 4 |
| agonist | cells | (±)DOI | | | (1 μM) | RT | | |

hr = human recombinant
1. Bonhaus et al., 1995, Brit. J. Pharmacol. 115: 622-628.
2. Bryant et al., 1996, Life Sci. 15: 1259-1268.
3. Kursar et al., 1994, Mol. Pharmacol. 46: 227-234.
4. Choi et al., 1994, FEBS Lett. 352: 393-399.

The results are expressed as a percent of control specific binding ((measured specific binding/control specific binding)×100) obtained in the presence of the test compounds, The IC₅₀ values (concentration causing a half-maximal inhibition of control specific binding) and Hill coefficients (nH) were determined by non-linear regression analysis of the competition curves generated with mean replicate values using Hill equation curve fitting (Y=D+[(A−D)/1+(C/C₅₀)^{nH})], where Y=specific binding, D=minimum specific binding, A=maximum specific binding, C=compound concentration, C₅₀=IC₅₀, and nH=slope factor). The percent inhibition of control specific binding is reported at 2 concentrations of 1.0E-07 and 1.0E-05 for test compounds for the 5-HT_{2A} and 5-HT_{2B} receptors as listed in Tables 9-12.

TABLE 9
Serotonin receptor 5-HT_{2A} binding (antagonist radioligand)

| | | % Inhibition of control | |
|---|---|---|---|
| | | specific binding | |
| Assay | Test compound | 1.0E−07 M | 1.0E−05 M |
| 5-HT_{2A} (h) | ICI-735 | 44 | 106 |
| (antagonist radioligand) | Compound 36a | 100 | 100 |
| | ICI-737 | 101 | 102 |
| | ICI-748 | 101 | 102 |
| | Compound 37b | 102 | 102 |

TABLE 10
Serotonin receptor 5-HT_{2A} binding (agonist radioligand)

| | | % Inhibition of control | |
|---|---|---|---|
| | | specific binding | |
| Assay | Test compound | 1.0E−07 M | 1.0E−05 M |
| 5-HT_{2A} (h) | ICI-735 | na | na |
| (agonist radioligand) | Compound 36a | 95 | 99 |
| | ICI-737 | 93 | 99 |
| | ICI-748 | 93 | 99 |
| | Compound 37b | 95 | 98 |

TABLE 11
Serotonin receptor 5-HT_{2B} binding (antagonist radioligand)

| | | % Inhibition of control | |
|---|---|---|---|
| | | specific binding | |
| Assay | Test compound | 1.0E−07 M | 1.0E−05 M |
| 5-HT_{2B} (h) | ICI-735 | na | na |
| (antagonist radioligand) | Compound 36a | 97 | 99 |
| | ICI-737 | 96 | 99 |
| | ICI-748 | 91 | 99 |
| | Compound 37b | 95 | 99 |

TABLE 12
Serotonin receptor 5-HT_{2B} binding (agonist radioligand)

| | | % Inhibition of control | |
|---|---|---|---|
| | | specific binding | |
| Assay | Test compound | 1.0E−07 M | 1.0E−05 M |
| 5-HT_{2B} (h) | ICI-735 | na | na |
| (agonist radioligand) | Compound 36a | 92 | 100 |
| | ICI-737 | 84 | 98 |
| | ICI-748 | 78 | 95 |
| | Compound 37b | 85 | 93 |

A full serotonin receptor binding panel was performed on select test compounds. The human cellular source for these serotonin binding assays, the specific radioligands and the assay conditions are listed in Table 13. The results for test compounds are presented in Table 14.

TABLE 13

| | | | | | Non- | | | |
|---|---|---|---|---|---|---|---|---|
| Assay | Source | Ligand | Conc | K_{d} | specific | Incub. | Detection | Bibl |
| 5-HT_{1A} (h) | hr HEK- | [³H]8-OH- | 0.3 nM | 0.5 nM | 8-OH-DPAT | 60 min, RT | Scintillation | 5 |
| agonist | 293 cells | DPAT | | | (10 μM) | | | |
| 5-HT_{1B} | rat | [¹²⁵I]CYP | 0.1 nM | 0.16 nM | serotonin | 120 min, | Scintillation | 6 |
| antagonist | cerebral | (+30 μM | | | (10 μM) | 37° C. | | |
| | cortex | isoproterenol) | | | | | | |
| 5-HT_{1D} | rat | [³H] | 1 nM | 0.5 nM | serotonin | 60 min, RT | Scintillation | 7 |
| agonist | recomb | serotonin | | | (10 μM) | | | |
| | CHO cells | | | | | | | |
| 5-HT_{2C} (h) | hr HEK- | [³H] | 1 nM | 0.5 nM | RS 102221 | 120 min, | Scintillation | 8 |
| antagonist | 293 cells | mesulergine | | | (10 μM) | 37° C. | | |
| 5-HT_{2C} (h) | hr HEK- | [¹²⁵I] | 0.1 nM | 0.9 nM | (±)DOI | 60 min, | Scintillation | 2 |
| agonist | 293 cells | (±)DOI | | | (10 μM) | 37° C. | | |
| 5-HT₄ₑ (h) | hr CHO | [³H] | 0.3 nM | 0.15 nM | serotonin | 60 min, | Scintillation | 9 |
| antagonist | cells | GR 113808 | | | (100 μM) | 37° C. | | |
| 5-HT₅ₐ (h) | hr HEK- | [³H] | 1.5 nM | 1.5 nM | serotonin | 120 min, | Scintillation | 10 |
| agonist | 293 cells | LSD | | | (100 μM) | 37° C. | | |
| 5-HT₆ (h) | hr CHO | [³H] | 2 nM | 1.8 nM | serotonin | 120 min, | Scintillation | 11 |
| agonist | cells | LSD | | | (100 μM) | 37° C. | | |
| 5-HT₇ (h) | hr CHO | [³H] | 4 nM | 2.3 nM | serotonin | 120 min, | Scintillation | 12 |
| agonist | cells | LSD | | | (10 μM) | 37° C. | | |
| 5-HT₃ (h) | hr CHO | [³H] | 0.5 nM | 1.15 nM | MDL 72222 | 120 min, | Scintillation | 13 |
| antagonist | cells | BRL 43694 | | | (10 μM) | RT | | |

5. Mulheron et al., 1994, J. Biol. Chem. 269: 12954-12962.
6. Hoyer et al., 1985, Eur. J. Pharmacol. 118: 1-12.
7. Wurch et al., 1997, J. Neurochem. 68: 410-418.
8. Stam et al., 1994, Eur. J. Pharmacol. 269: 339-348.
9. Mial et al., 2000, Brit. J. Pharmacol. 129: 771-781.
10. Rees et al., 1994, FEBS Lett. 355: 242-246.
11. Monsma et al., 1993, Mol. Pharmacol. 43: 320-327.
12. Shen et al., 1993, J. Biol. Chem. 268: 18200-18204.
13. Hope et al., 1996, Brit. J. Pharmacol. 118: 1237-1245.

TABLE 14
Serotonin receptor binding profile

| | | % Inhibition of control | |
|---|---|---|---|
| | | specific binding | |
| Assay | Test compound | 1.0E−07 M | 1.0E−05 M |
| 5-HT_{1A} (h) | Compound 36a | 24 | 86 |
| (agonist radioligand) | ICI-735 | 20 | 95 |
| 5-HT_{1B} | Compound 36a | 41 | 90 |
| (antagonist radioligand) | ICI-735 | 4 | 78 |
| 5-HT_{1D} | Compound 36a | 30 | 67 |
| (agonist radioligand) | ICI-735 | −5 | 25 |
| 5-HT_{2C} (h) | Compound 36a | 31 | 93 |
| (antagonist radioligand) | ICI-735 | 9 | 71 |
| 5-HT_{2C} (h) | Compound 36a | 22 | 95 |
| (agonist radioligand) | ICI-735 | na | na |
| 5-HT₃ (h) | Compound 36a | 0 | 21 |
| (antagonist radioligand) | ICI-735 | 19 | 13 |
| 5-HT₄ₑ (h) | Compound 36a | 6 | 41 |
| (antagonist radioligand) | ICI-735 | 32 | 72 |
| 5-HT₅ₐ (h) | Compound 36a | 34 | 96 |
| (agonist radioligand) | ICI-735 | 8 | 92 |
| 5-HT₆ (h) | Compound 36a | 90 | 101 |
| (agonist radioligand) | ICI-735 | 22 | 97 |
| 5-HT₇ (h) | Compound 36a | 85 | 100 |
| (agonist radioligand) | ICI-735 | 69 | 101 |

### Example 41

### Metabolism Assays—Hepatocyte Stability Assay

Test compounds (5 μM) were incubated with cryopreserved mixed gender human hepatocytes pooled from at least 3 donors. Cell viability of hepatocytes was assessed by a Trypan Blue assay prior to the initiation of the stability assay. The final hepatocyte cell density was 1.5×10⁶ viable cells/mL, Samples were taken at 2 time points: 0 and 60 minutes. As positive controls, testosterone (20 μM) and 7-hydroxy-coumarin (100 μM) were incubated and sampled at 5 time points: 0, 15, 30, 60, and 120 minutes. Aliquots were removed and combined (50/50) with acetonitrile to terminate the reaction. Samples were mixed for 10 minutes and centrifuged. The supernatants were transferred to vial for analysis by HRMS. In this assay, both test compound and a des-glycine metabolite (M1) are quantified based on peak area response ratios.

The enzymatic activities of the human cryopreserved hepatocytes used in this study were verified in parallel by determining the disappearance of testosterone (expressed as half-life) and the formation of 7-hydroxycoumarin glucuronide and 7-hydroxycoumarin sulfate (expressed as a formation rate).

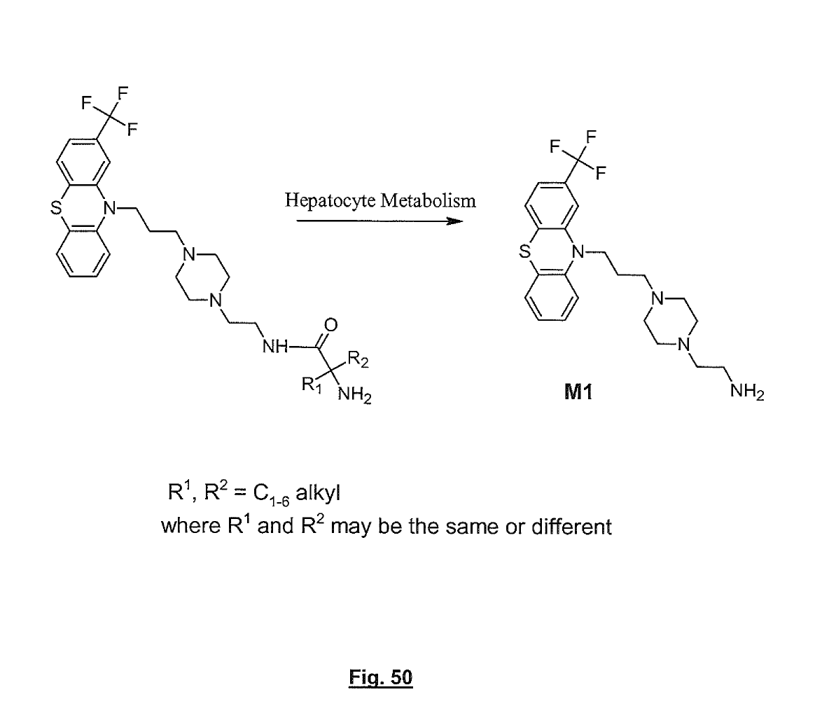

The comparative hepatocyte stability shown in Table 15 indicate that Compound 36a is less susceptible to the metabolic transformation to the metabolite, M1 (FIG. 50) than ICI-735. This metabolic protection is attributed to the increased steric hindrance associated with alkyl substitution of the alpha position in the glycyl unit to attack on the acetamide carbonyl by external nucleophiles.

TABLE 15
Hepatocyte Stability Data

| | Relative Level (%) | | Peak Areas at |
|---|---|---|---|
| Analyte | T = 0 minutes | T = 60 minutes | T = 60 minutes |
| ICI-735 | 99.5 | 89.6 | 8.39E+06 |
| | 100.5 | 92.4 | 8.79E+06 |
| M1 from ICI-735 | NF | 17.3 | 1.62E+06 |
| | NF | 15.5 | 1.48E+06 |
| Compound 36a | 97.3 | 99.0 | 3.42E+06 |
| | 102.8 | 109.1 | 3.82E+06 |
| M1 from | 0.1 | 0.4 | 1.27E+04 |
| Compound 36a | 0.1 | 0.4 | 1.25E+04 |

### Example 42

### Phospholipidosis Assay

The Food and Drug Administration (FDA) has acknowledged that drug-induced phospholipidosis is an adverse drug reaction that warrants both additional guidelines and research into the molecular mechanisms that govern this biological response (Berridge et al., 2007, Toxicol. Pathol. 35:325). Detection of drug-induced phospholipidosis has been performed using electron microscopy, a time-consuming and labor intensive technique, and/or quantitative PCR.

More recently, fluorescent dyes have been utilized to assess phospholipidosis in a high throughput manner (Nioi et al., 2007, Toxicol. Sci. 99:162-173). In this assay, HepG2 cells are plated in MEM growth medium and allowed to incubate overnight. Cells are treated with test compounds that have been added to the assay medium (10% fetal bovine serum) containing LipidTox (fluorescent lipophilic dye). After 48 h incubation, cells are fixed and stained with Hoechst. Plates are scanned with an automated fluorescent microscope (Thermo Fisher Cellomics ArrayScan 4.5) and image analysis is used to quantitate cell number and phospholipid accumulation. Compounds are tested in triplicate at multiple concentrations. Three reference compounds, sertraline, perhexyline, and meclizine (high, medium, and low inducers of phospholipidosis, respectively) are included in each assay. The data is expressed as fold induction over background and the % of the positive control (sertraline), which is calculated using the following equation:

% positive control=100×(RFUcompound−RFUbackground)/(RFUsertaline−RFUbackground),

where RFU=Relative Fluorescence Unit

TABLE 16

| | | Fold | % Positive | % |
|---|---|---|---|---|
| Test Compound | Conc (μM) | Induction | Control | Cytotoxicity |
| ICI-735 | 1 | 23 | 43 | 0 |
| | 3 | 6 | 10 | 68 |
| | 10 | NA | NA | 100 |
| | 30 | NA | NA | 100 |
| Compound 37b | 1 | 1 | 0.2 | 32 |
| | 3 | 4 | 5.8 | 17 |
| | 10 | 12 | 21.3 | 46 |
| | 30 | | | 100 |
| Sertaline | 3 | 52 | 100 | 15 |
| Perhexil | 3 | 38 | 72 | 10 |
| Meclizine | 25 | 13 | 24 | 0 |

These studies indicate that neutralization or capping of the terminal cationic groups in serotonin antagonists, such as in Compound 37b, causes the compounds to have lower propensity to form phospholipidosis and to produce less cytotoxicity to HepG2 cells.

### Example 43

### Monocrotaline Model of Pulmonary Arterial Hypertension (PAH)

### Experimental Design

Adult male Sprague-Dawley rats (287±4 g) were obtained from Charles River Laboratories (Raleigh, N.C.). Animals housed individually in a temperature/humidity controlled room with 12-hour light/dark cycles had free access to water and food and were acclimated for one week prior to the study. All experimental protocols were approved by the University of Illinois at Chicago Care and Use Committee, and all experiments were conducted in accordance with the NIH guidelines for animal welfare.

Rats were randomly assigned to one of five experimental groups (n=10 per group). Rats in groups 1 and 2 served as healthy controls; the remaining rats were injected subcutaneously on Day 0 with 60 mg/kg body weight monocrotaline, the toxic alkaloid of C. spectabilis (dissolved in DMSO at a concentration of 60 mg/mL, Sigma Aldrich, St. Louis, Mo.). On days 1-21, rats were dosed via oral gavage (2 mL/kg) with vehicle (PBS), or ICI-735 at 1 mg/kg or 10 mg/kg. Rats were weighed daily, and the dosages were adjusted appropriately.

On day 21, the animals were anesthetized by intra-muscular injection of ketamine/xylazine (80/10 mg/kg) and placed on a heating pad to maintain body temperature at 37° C. A Millar catheter 1.4 French (Millar Instruments, Houston, Tex.) was inserted into the femoral artery to measure arterial blood pressure. Additionally, the pulmonary artery and right ventricular (RV) pressures were measured as described previously (Stinger et al., 1981), Briefly, a 3.5 French umbilical vessel catheter (Utah Medical Products LTD, Midvale, Utah), angled to 90° over the distal 1 cm and curved slightly at the tip, was introduced into the right external jugular vein, with the angle directed interiorly, the catheter was inserted proximally, which placed the catheter in the right atrium. The catheter was rotated 90° counterclockwise and inserted further, which placed the catheter in the right ventricle, and then advanced approximately 1.5 cm, into the pulmonary artery. Placement at each stage was confirmed by monitoring the respective pressure contours. Hemodynamic values were automatically calculated by the physiological data acquisition system NOTOCORD-Hem Software 4.1 (NOTOCORD Inc., Kalamazoo, Mich.).

At the end of the study, rats were euthanized by pentobarbital overdose and hearts were isolated, flushed with saline and dissected to separate the right ventricle from the left ventricle+septum (LV+S). Dissected samples were weighed and the ratio of the RV weight to body weight [RV/BW] for each heart was calculated to obtain an index of RV hypertrophy.

After the lungs were harvested, they were instilled with 10% neutral buffered formalin and immersed in the same fixative. The left and right caudal lung lobes were trimmed to produce six transverse samples per rat and these samples were routinely processed and embedded in paraffin blocks. Sections (approximately 5 μm thick) were stained with Verhoeff's elastin/eosin stain and examined by light microscopy, Histopathological findings were classified as: 1-alveolar inflammation and septal remodeling, 2-perivascular inflammation and edema, 3-perivascular fibrosis, and 4-arteriolar medial hypertrophy. The findings were graded by a pathologist without knowledge of treatment group assignment as 0 (not present), 1 (minimal), 2 (mild), 3 (moderate), or 4 (marked).

The distribution of each finding, if present was classified as multifocal or diffuse. The degree of muscularization of small peripheral pulmonary arteries was assessed by examination of sections immunohistochemically reacted with an anti-alpha-smooth muscle actin antibody (rabbit polyclonal ab5694 diluted 1:100, Abeam, Cambridge, Mass.).

These sections were stained with Verhoeff's elastin stain and examined by light microscopy with the aid of an eyepiece micrometer. Eighty intra-acinar pulmonary arterioles with diameter of 10 to 50 μn were categorized as non-muscularized (exhibit elastin but no apparent smooth muscle), partially-muscularized (incomplete medial layer of smooth muscle), or fully-muscularized (concentric medial layer of smooth muscle) (Schermuly et al., 2004). The percentage of pulmonary vessels in each muscularization category was determined for each rat.

### Results

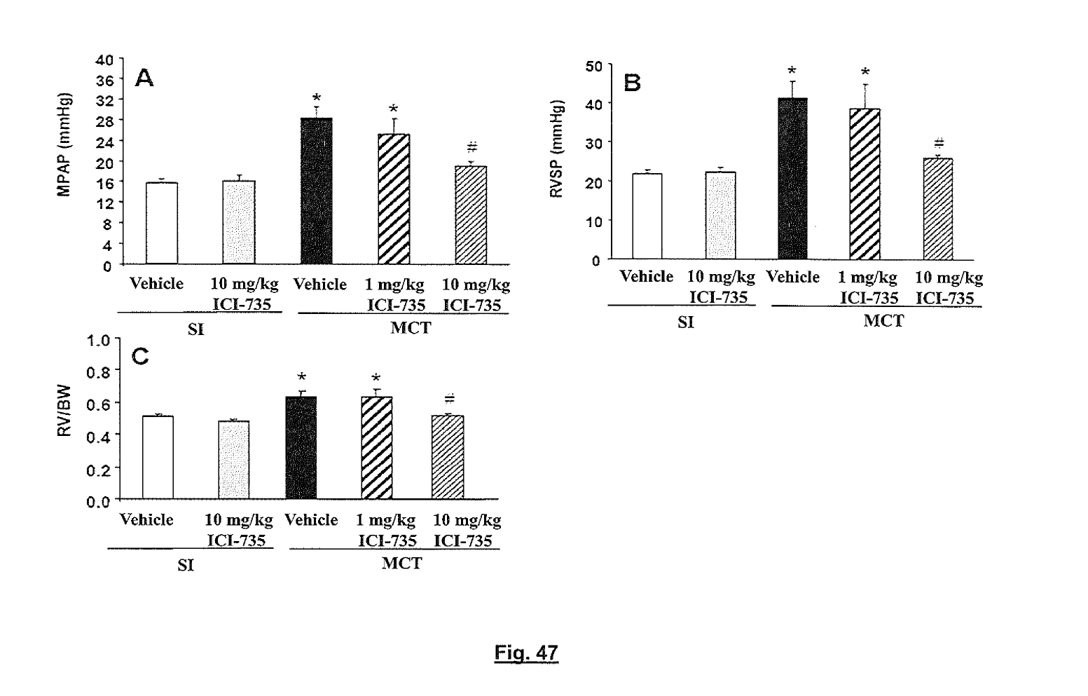

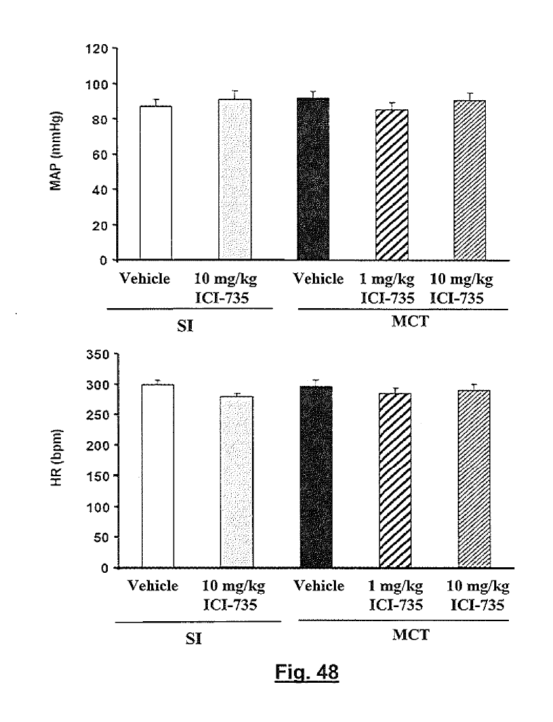

Daily oral treatment of MCT rats with ICI-735 at 10 mg/kg for 21 days reduced MCT-induced elevations of PAP, RVSP, and RV/BW by 75%, 78% and 81%, respectively (p<0.05, FIGS. 47A-47C). At a dose of 1 mg/kg, ICI-735 did not attenuate the effects of MCT on PAP, RVSP and RV/BW (FIGS. 47A-47C). SI rats exposed to 10 mg/kg ICI-735 exhibited no changes in PAP, RVSP or RV/BW compared with vehicle controls (FIG. 47A-47C). Mean arterial pressure (MAP) and heart rate (HR) were unmodified compared with controls in all ICI-735-treated groups (FIG. 48). Daily clinical evaluation showed no evidence of physical or behavioral drug-related toxicity.

Microscopic evaluation of lungs from MCT/vehicle rats revealed alveolar inflammation and septal remodeling, perivascular inflammation and edema, perivascular fibrosis, and arteriolar medial hypertrophy as indicated by greater incidences and severity scores for all parameters evaluated as compared with SI/vehicle controls (Table 17). MCT rats treated with 10 mg/kg ICI-735 had a marked decrease in the incidences and severities of perivascular fibrosis and arteriolar medial hypertrophy (Table 17). The severities of alveolar inflammation and septal remodeling, and perivascular inflammation and edema were also clearly diminished in the MCT rats treated with 10 mg/kg ICI-735 as compared to the MCT/vehicle group (Table 17).

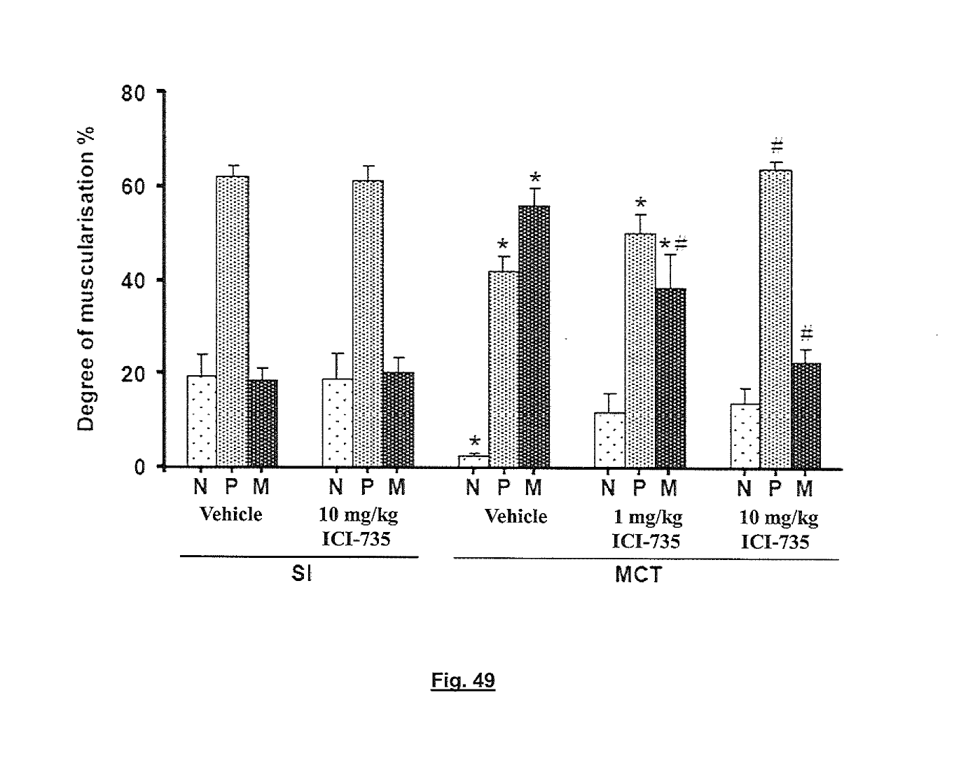

Categorization of 10 to 50 μm diameter pulmonary arterioles as fully, partially, or nonmuscularized revealed a 3-fold increase in completely muscularized arterioles and 8.1-fold and 1.5-fold decreases in non-muscularized and partially muscularized arterioles, respectively, in MCT-vehicle rat lungs at day 21 compared with SI-vehicle controls (FIG. 49). In contrast, MCT rats treated with high-dose ICI-735 exhibited no significant differences in the degree of muscularization of pulmonary arterioles compared with SI/vehicle controls (FIG. 49).

TABLE 17
Pulmonary histopathology incidence from saline-injected
control (SI) and MCT-injected (MCT) rats receiving
vehicle, 10 mg/kg or 1 mg/kg ICI-735 for 21 days

| | Dose group (n = 10) | | | | |
|---|---|---|---|---|---|
| | | SI + | | MCT + | MCT + |
| | SI + | 10 mg/kg | MCT + | 1 mg/kg | 10 mg/kg |
| Pulmonary lesion | vehicle | ICI-735 | vehicle | ICI-735 | ICI-735 |

Alveolar inflammation/septal remodeling

| | | | | | | |
|---|---|---|---|---|---|---|
| Severity score | 0 | 2 | 1 | — | — | — |
| (0-4)^{a} | 1 | 8 | 9 | 2 | 3 | 6 |
| | 2 | — | — | 5 | 4 | 3 |
| | 3 | — | — | 2 | 1 | 1 |
| | 4 | — | — | 1 | 2 | — |

Perivascular inflammation/edema

| | | | | | | |
|---|---|---|---|---|---|---|
| Severity score | 0 | 3 | 4 | — | — | — |
| (0-4)^{a} | 1 | 6 | 5 | 4 | 4 | 8 |
| | 2 | 1 | 1 | 6 | 5 | 2 |
| | 3 | — | — | — | 1 | — |
| | 4 | — | — | — | — | — |

Perivascular flbrosis

| | | | | | | |
|---|---|---|---|---|---|---|
| Severity score | 0 | 10 | 10 | 1 | 2 | 7 |
| (0-4)^{a} | 1 | — | — | 8 | 5 | 3 |
| | 2 | — | — | 1 | 3 | — |
| | 3 | — | — | — | — | — |
| | 4 | — | — | — | — | — |

Arteriolar medial hypertrophy

| | | | | | | |
|---|---|---|---|---|---|---|
| Severity score | 0 | 7 | 10 | — | — | 3 |
| (0-4)^{a} | 1 | 3 | — | — | 1 | 5 |
| | 2 | — | — | 3 | 6 | 2 |
| | 3 | — | — | 7 | 3 | — |
| | 4 | — | — | — | — | — |

^{a}Severity of lesions was scored as follows:
0 = finding not present, 1 = minimal, 2 = mild, 3 = moderate, 4 = marked

The disclosures of each and every patent, patent application, and publication cited herein are hereby incorporated herein by reference in their entirety.

While the invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A composition comprising a compound of formula II or a salt thereof: 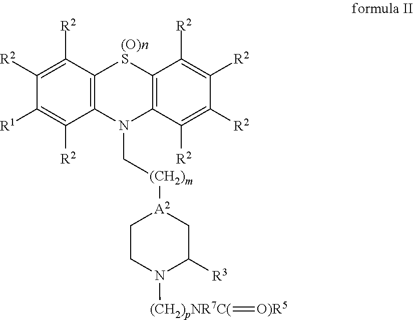
wherein:
each occurrence of R¹ and R² is independently selected from the group consisting of hydrogen, halogen, (C₁-C₆)alkyl; (C₁-C₆)alkenyl; (C₁-C₆)alkoxy; OH; NO₂; C≡N; C(═O)OR⁷; C(═O)NR⁷₂; NR⁷₂; NR⁷C(═O)(C₁-C₆)alkyl; NR⁷C(═O)O(C₁-C₆)alkyl; NR⁷C(═O)NR⁷₂; NR⁷SO₂(C₁-C₆)alkyl; SO₂NR⁷₂; OC(═O)(C₁-C₆)alkyl; O(C₂-C₆)alkylene-NR⁷₂; (C₂-C₆)alkylene-OR⁷; and (C₁-C₃)perfluoroalkyl;
R³ is hydrogen, C(═O)OR⁷, or C(═O)NR⁷₂;
A² is CH or N;
R⁵ is H or CR⁸R⁹R¹⁰;
each occurrence of R⁷ and R¹⁰ is independently selected from the group consisting of hydrogen, (C₁-C₆)cycloalkyl and (C₁-C₆)alkyl;
each occurrence of R⁸ and R⁹ is independently selected from the group consisting of (C₁-C₆)cycloalkyl and (C₁-C₆)alkyl; or R⁸ and R⁹ are bound to the same carbon atom and linked as to form a divalent group selected from the group consisting of ethane-1,2-diyl, propane-1,3-diyl, butane-1,4-diyl, pentane-1,5-diyl, hexane-1,6-diyl and heptane-17-diyl; wherein said bivalent group is optionally substituted with at least one (C₁-C₆)alkyl group;
m is independently at each occurrence 1, 2, or 3;
n is 0, 1, or 2;
p is independently at each occurrence 2 or 3; and
q is independently at each occurrence 1 or 2.

2. The composition of claim 1, wherein R² is hydrogen.

3. The composition of claim 1, wherein R³ is hydrogen.

4. The composition of claim 1, wherein A² is N.

5. The composition of claim 1, wherein R⁵ is C(CH₃)₃.

6. The composition of claim 1, wherein m is 2, n is 0, p is 2, and q is 1.

7. The composition of claim 1, wherein said compound is N-(2-(4-(3-(2-(trifluoromethyl)-10H-phenothiazin-10-yl)propyl)piperazin-1-yl)ethyl)pivalamide (Compound 35b) or a salt thereof.

8. A composition comprising a compound of formula III or a salt thereof: 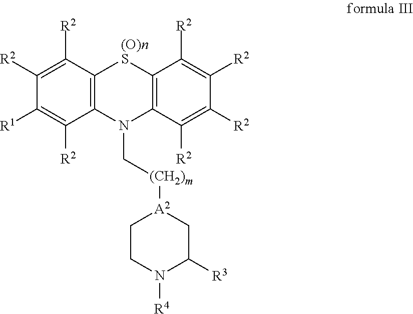
wherein:
each occurrence of R¹ and R² is independently selected from the group consisting of hydrogen, halogen, (C₁-C₆)alkyl; (C₁-C₆)alkenyl; (C₁-C₆)alkoxy; OH; NO₂; C≡N; C(═O)OR⁵; C(═O)NR⁵₂; NR⁵₂; NR⁵C(═O)(C₁-C₆)alkyl; NR⁵C(═O)O(C₁-C₆)alkyl; NR⁵C(═O)NR⁵₂; NR⁵SO₂(C₁-C₆)alkyl; SO₂NR⁵₂; OC(═O)(C₁-C₆)alkyl; O(C₂-C₆)alkylene-NR⁵₂; (C₂-C₆)alkylene-OR⁵; and (C₁-C₃)perfluoroalkyl;
R³ is hydrogen, C(═O)OR⁵, or C(═O)N(R⁵)₂;
A² is CH or N;
R⁴ is —(CR⁵₂)ₚN(R⁵)C(═O)—CR⁶R⁷R⁸; —(CR⁵₂)ₚO(CR⁵₂)ₚN(R⁵)C(═O)—CR⁶R⁷R⁸; —(CR⁵₂)ₚN(R⁵)(CR⁵₂)ₚN(R⁵)C(═O)—CR⁶R⁷R⁸; or —(CR⁵₂)ₚN(R⁵)C(═O)(CR⁵₂)ₚN(R⁵)C(═O)—CR⁶R⁷R⁸;
each occurrence of R⁵ and R⁶ is independently selected from the group consisting of hydrogen, (C₁-C₆)alkyl and (C₁-C₆)cycloalkyl;
R⁷ is (C₁-C₆)alkyl or (C₁-C₆)cycloalkyl; or R⁶ and R⁷ are bound to the same carbon atom and linked as to form a divalent group selected from the group consisting of ethane-1,2-diyl, propane-1,3-diyl, butane-1,4-diyl, pentane-1,5-diyl, hexane-1,6-diyl and heptane-17-diyl; wherein said bivalent group is optionally substituted with at least one (C₁-C₆)alkyl group;
R⁸ is (C₁-C₆)alkyl, —N(R⁵)C(═O)R⁵, or —N(R⁵)S(═O)₂R⁷;
m is independently at each occurrence 1, 2, or 3;
n is 0, 1, or 2; and,
p is independently at each occurrence 1, 2 or 3.

9. The composition of claim 8, wherein R³ is hydrogen.

10. The composition of claim 8, wherein A² is N.

11. The composition of claim 8, wherein R⁴ is —(CR⁵₂)ₚN(R⁵)C(═O)—CR⁶R⁷R⁸.

12. The composition of claim 8, wherein m is 2, n is 0, p is 2, and q is 1.

13. The composition of claim 8, wherein said compound is selected from the group consisting of 2-amino-2-methyl-N-(2-(4-(3-(2-(trifluoromethyl)-10H-phenothiazin-10-yl)propyl)piperazin-1-yl)ethyl)propanamide (Compound 36a), 2-formamido-N-(2-(4-(3-(2-(trifluoromethyl)-10H-phenothiazin-10-yl)propyl)piperazin-1-yl)ethyl)acetamide (Compound 37b), a salt thereof, and mixtures thereof.

14. A method of inducing apoptosis in an immune cell or lymphocyte, said method comprising contacting said immune cell or lymphocyte with a composition comprising a compound selected from the group comprising:
a compound of formula II: 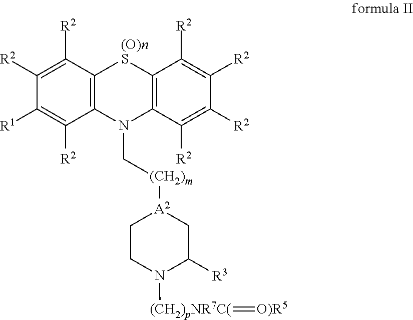
wherein:
each occurrence of R¹ and R² is independently selected from the group consisting of hydrogen, halogen, (C₁-C₆)alkyl; (C₁-C₆)alkenyl; (C₁-C₆)alkoxy; OH; NO₂; C≡N; C(═O)OR⁷; C(═O)NR⁷₂; NR⁷₂; NR⁷C(═O)(C₁-C₆)alkyl; NR⁷C(═O)O(C₁-C₆)alkyl; NR⁷C(═O)NR¹₂; NR⁷SO₂(C₁-C₆)alkyl; SO₂NR⁷₂; OC(═O)(C₁-C₆)alkyl; O(C₂-C₆)alkylene-NR⁷₂; (C₂-C₆)alkylene-OR⁷; and (C₁-C₃)perfluoroalkyl;
R³ is hydrogen, C(═O)OR⁷, or C(═O)NR⁷₂;
A² is CH or N;
R⁵ is H or CR⁸R⁹R¹⁰;
each occurrence of R⁷ and R¹⁰ is independently selected from the group consisting of hydrogen, (C₁-C₆)cycloalkyl and (C₁-C₆)alkyl;
each occurrence of R⁸ and R⁹ is independently selected from the group consisting of (C₁-C₆)cycloalkyl and (C₁-C₆)alkyl; or R⁸ and R⁹ are bound to the same carbon atom and linked as to form a divalent group selected from the group consisting of ethane-1,2-diyl, propane-1,3-diyl, butane-1,4-diyl, pentane-1,5-diyl, hexane-1,6-diyl and heptane-17-diyl; wherein said bivalent group optionally substituted is with at least one (C₁-C₆)alkyl group;
m is independently at each occurrence 1, 2, or 3;
n is 0, 1, or 2;
p is independently at each occurrence 2 or 3; and
q is independently at each occurrence 1 or 2;
a compound of formula III: 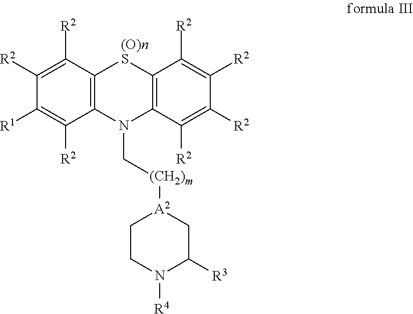
wherein:
each occurrence of R¹ and R² is independently selected from the group consisting of hydrogen, halogen, (C₁-C₆)alkyl; (C₁-C₆)alkenyl; (C₁-C₆)alkoxy; OH; NO₂; C≡N; C(═O)OR⁵; C(═O)NR⁵₂; NR⁵₂; NR⁵C(═O)(C₁-C₆)alkyl; NR⁵C(═O)O(C₁-C₆)alkyl; NR⁵C(═O)NR⁵₂; NR⁵SO₂(C₁-C₆)alkyl; SO₂NR⁵₂; OC(═O)(C₁-C₆)alkyl; O(C₂-C₆)alkylene-NR⁵₂; (C₂-C₆)alkylene-OR⁵; and (C₁-C₃)perfluoroalkyl;
R³ is hydrogen, C(═O)OR⁵, or C(═O)N(R⁵)₂;
A² is CH or N;
R⁴ is —(CR⁵₂)ₚN(R⁵)C(═O)—CR⁶R⁷R⁸; —(CR⁵₂)ₚO(CR⁵₂)ₚN(R⁵)C(═O)—CR⁶R⁷R⁸; —(CR⁵₂)ₚN(R⁵)(CR⁵₂)ₚN(R⁵)C(═O)—CR⁶R⁷R⁸; or —(CR⁵₂)ₚN(R⁵)C(═O)(CR⁵₂)ₚN(R⁵)C(═O)—CR⁶R⁷R⁸;
each occurrence of R⁵ and R⁶ is independently selected from the group consisting of hydrogen, (C₁-C₆)alkyl and (C₁-C₆)cycloalkyl;
R⁷ is (C₁-C₆)alkyl or (C₁-C₆)cycloalkyl; or R⁶ and R⁷ are bound to the same carbon atom and linked as to form a divalent group selected from the group consisting of ethane-1,2-diyl, propane-1,3-diyl, butane-1,4-diyl, pentane-1,5-diyl, hexane-1,6-diyl and heptane-17-diyl; wherein said bivalent group is optionally substituted with at least one (C₁-C₆)alkyl group;
R⁸ is (C₁-C₆)alkyl, —N(R⁵)C(═O)R⁵, or —N(R⁵)S(═O)₂R⁷;
m is independently at each occurrence 1, 2, or 3;
n is 0, 1, or 2; and,
p is independently at each occurrence 1, 2 or 3;
a salt thereof and mixtures thereof, thereby inducing apoptosis in said immune cell or lymphocyte.

15. The method of claim 14, wherein said lymphocyte is selected from the group consisting of a T cell and a B cell.

16. The method of claim 15, wherein said B cell is a plasma cell.

17. The method of claim 16, wherein said plasma cell is a multiple myeloma cell.

18. A method of inhibiting proliferation of a lymphocyte, said method comprising contacting said lymphocyte with a composition comprising a compound selected from the group comprising:
a compound of formula H: 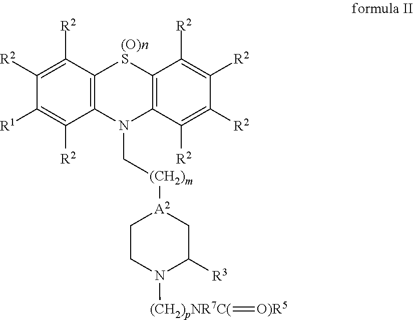
wherein:
each occurrence of R¹ and R² is independently selected from the group consisting of hydrogen, halogen, (C₁-C₆)alkyl; (C₁-C₆)alkenyl; (C₁-C₆)alkoxy; OH; NO₂; C≡N; C(═O)OR⁷; C(═O)NR⁷₂; NR⁷₂; NR⁷C(═O)(C₁-C₆)alkyl; NR⁷C(═O)O(C₁-C₆)alkyl; NR⁷C(═O)NR⁷₂; NR⁷SO₂(C₁-C₆)alkyl; SO₂NR⁷₂; OC(═O)(C₁-C₆)alkyl; O(C₂-C₆)alkylene-NR⁷₂; (C₂-C₆)alkylene-OR⁷; and (C₁-C₃)perfluoroalkyl;
R³ is hydrogen, C(═O)OR⁷, or C(═O)NR⁷₂;
A² is CH or N;
R⁵ is H or CR⁸R⁹R¹⁰;
each occurrence of R⁷ and R¹⁰ is independently selected from the group consisting of hydrogen, (C₁-C₆)cycloalkyl and (C₁-C₆)alkyl;
each occurrence of R⁸ and R⁹ is independently selected from the group consisting of (C₁-C₆)cycloalkyl and (C₁-C₆)alkyl; or R⁸ and R⁹ are bound to the same carbon atom and linked as to form a divalent group selected from the group consisting of ethane-1,2-diyl, propane-1,3-diyl, butane-1,4-diyl, pentane-1,5-diyl, hexane-1,6-diyl and heptane-17-diyl; wherein said bivalent group is optionally substituted with at least one (C₁-C₆)alkyl group;
m is independently at each occurrence 1, 2, or 3;
n is 0, 1, or 2;
p is independently at each occurrence 2 or 3; and
q is independently at each occurrence 1 or 2;
a compound of formula III: 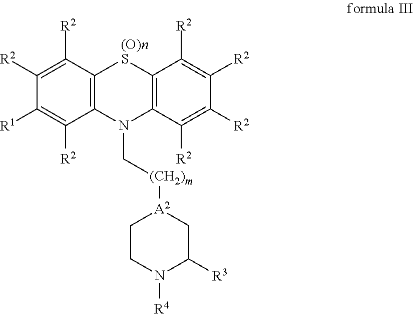
wherein:
each occurrence of R¹ and R² is independently selected from the group consisting of hydrogen, halogen, (C₁-C₆)alkyl; (C₁-C₆)alkenyl; (C₁-C₆)alkoxy; OH; NO₂; C≡N; C(═O)OR⁵; C(═O)NR⁵₂; NR⁵₂; NR⁵C(═O)(C₁-C₆)alkyl; NR⁵C(═O)O(C₁-C₆)alkyl; NR⁵C(═O)NR⁵₂; NR⁵SO₂(C₁-C₆)alkyl; SO₂NR⁵₂; OC(═O)(C₁-C₆)alkyl; O(C₂-C₆)alkylene-NR⁵₂; (C₂-C₆)alkylene-OR⁵; and (C₁-C₃)perfluoroalkyl;
R³ is hydrogen, C(═O)OR⁵, or C(═O)N(R⁵)₂;
A² is CH or N;
R⁴ is —(CR⁵₂)ₚN(R⁵)C(═O)—CR⁶R⁷R⁸; —(CR⁵₂)ₚO(CR⁵₂)ₚN(R⁵)C(═O)—CR⁶R⁷R⁸; —(CR⁵₂)ₚN(R⁵)(CR⁵₂)ₚN(R⁵)C(═O)—CR⁶R⁷R⁸; or —(CR⁵₂)ₚN(R⁵)C(═O)(CR⁵₂)ₚN(R⁵)C(═O)—CR⁶R⁷R⁸;
each occurrence of R⁵ and R⁶ is independently selected from the group consisting of hydrogen, (C₁-C₆)alkyl and (C₁-C₆)cycloalkyl;
R⁷ is (C₁-C₆)alkyl or (C₁-C₆)cycloalkyl; or R⁶ and R⁷ are bound to the same carbon atom and linked as to form a divalent group selected from the group consisting of ethane-1,2-diyl, propane-1,3-diyl, butane-1,4-diyl, pentane-1,5-diyl, hexane-1,6-diyl and heptane-17-diyl; wherein said bivalent group is optionally substituted with at least one (C₁-C₆)alkyl group;
R⁸ is (C₁-C₆)alkyl, —N(R⁵)C(═O)R⁵, or —N(R⁵)S(═O)₂R⁷;
m is independently at each occurrence 1, 2, or 3;
n is 0, 1, or 2; and,
p is independently at each occurrence 1, 2 or 3;
a salt thereof and mixtures thereof, thereby inhibiting proliferation of said lymphocyte.

19. The method of claim 18, wherein said lymphocyte is selected from the group consisting of a T cell and a B cell.

20. The method of claim 19, wherein said B cell is a plasma cell.

21. The method of claim 20, wherein said plasma cell is a multiple myeloma cell.

22. A method of treating a disease characterized by abnormal lymphocyte proliferation in a mammal, said method comprising administering to said mammal a therapeutically effective amount of a pharmaceutically acceptable composition comprising a compound selected from the group comprising:
a compound of formula II: 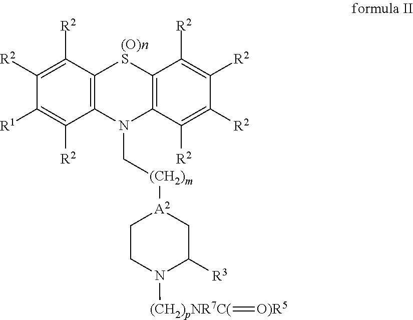
wherein:
each occurrence of R¹ and R² is independently selected from the group consisting of hydrogen, halogen, (C₁-C₆)alkyl; (C₁-C₆)alkenyl; (C₁-C₆)alkoxy; OH; NO₂; C≡N; C(═O)OR⁷; C(═O)NR⁷₂; NR⁷₂; NR⁷C(═O)(C₁-C₆)alkyl; NR⁷C(═O)O(C₁-C₆)alkyl; NR⁷C(═O)NR⁷₂; NR⁷SO₂(C₁-C₆)alkyl; SO₂NR⁷₂; OC(═O)(C₁-C₆)alkyl; O(C₂-C₆)alkylene-NR⁷₂; (C₂-C₆)alkylene-OR⁷; and (C₁-C₃)perfluoroalkyl;
R³ is hydrogen, C(═O)OR⁷, or C(═O)NR⁷₂;
A² is CH or N;
R⁵ is H or CR⁸R⁹R¹⁰;
each occurrence of R⁷ and R¹⁰ is independently selected from the group consisting of hydrogen, (C₁-C₆)cycloalkyl and (C₁-C₆)alkyl;
each occurrence of R⁸ and R⁹ is independently selected from the group consisting of (C₁-C₆)cycloalkyl and (C₁-C₆)alkyl; or R⁸ and R⁹ are bound to the same carbon atom and linked as to form a divalent group selected from the group consisting of ethane-1,2-diyl, propane-1,3-diyl, butane-1,4-diyl, pentane-1,5-diyl, hexane-1,6-diyl and heptane-17-diyl; wherein said bivalent group is optionally substituted with at least one (C₁-C₆)alkyl group;
m is independently at each occurrence 1, 2, or 3;
n is 0, 1, or 2;
p is independently at each occurrence 2 or 3; and
q is independently at each occurrence 1 or 2;
a compound of formula III: 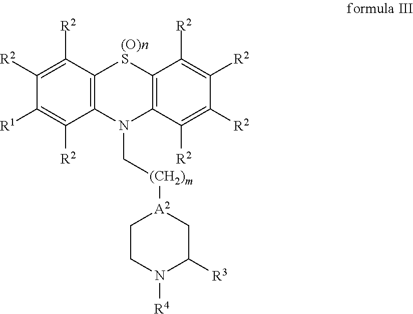
wherein:
each occurrence of R¹ and R² is independently selected from the group consisting of hydrogen, halogen, (C₁-C₆)alkyl; (C₁-C₆)alkenyl; (C₁-C₆)alkoxy; OH; NO₂; C≡N; C(═O)OR⁵; C(═O)NR⁵₂; NR⁵₂; NR⁵C(═O)(C₁-C₆)alkyl; NR⁵C(═O)O(C₁-C₆)alkyl; NR⁵C(═O)NR⁵₂; NR⁵SO₂(C₁-C₆)alkyl; SO₂NR⁵₂; OC(═O)(C₁-C₆)alkyl; O(C₂-C₆)alkylene-NR⁵₂; (C₂-C₆)alkylene-OR⁵; and (C₁-C₃)perfluoroalkyl;
R³ is hydrogen, C(═O)OR⁵, or C(═O)N(R⁵)₂;
A² is CH or N;
R⁴ is —(CR⁵₂)ₚN(R⁵)C(═O)—CR⁶R⁷R⁸; —(CR⁵₂)ₚO(CR⁵₂)ₚN(R⁵)C(═O)—CR⁶R⁷R⁸; —(CR⁵₂)ₚN(R⁵)(CR⁵₂)ₚN(R⁵)C(═O)—CR⁶R⁷R⁸; or —(CR⁵₂)ₚN(R⁵)C(═O)(CR⁵₂)ₚN(R⁵)C(═O)—CR⁶R⁷R⁸;
each occurrence of R⁵ and R⁶ is independently selected from the group consisting of hydrogen, (C₁-C₆)alkyl and (C₁-C₆)cycloalkyl;
R⁷ is (C₁-C₆)alkyl or (C₁-C₆)cycloalkyl; or R⁶ and R⁷ are bound to the same carbon atom and linked as to form a divalent group selected from the group consisting of ethane-1,2-diyl, propane-1,3-diyl, butane-1,4-diyl, pentane-1,5-diyl, hexane-1,6-diyl and heptane-17-diyl; wherein said bivalent group is optionally substituted with at least one (C₁-C₆)alkyl group;
R⁸ is (C₁-C₆)alkyl, —N(R⁵)C(═O)R⁵, or —N(R⁵)S(═O)₂R⁷;
m is independently at each occurrence 1, 2, or 3;
n is 0, 1, or 2; and,
p is independently at each occurrence 1, 2 or 3;
a salt thereof and mixtures thereof, thereby treating said disease in said mammal.

23. A method of treating a disease selected from the group consisting of asthma and rheumatoid arthritis in a mammal, said method comprising administering to said mammal a therapeutically effective amount of a pharmaceutically acceptable composition comprising a compound selected from the group comprising:
a compound of formula II: 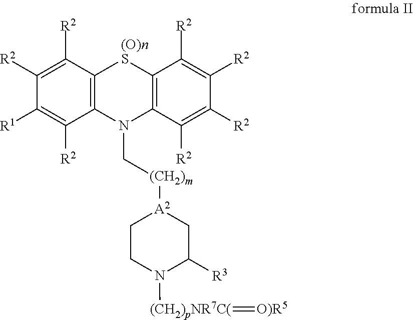
wherein:
each occurrence of R¹ and R² is independently selected from the group consisting of hydrogen, halogen, (C₁-C₆)alkyl; (C₁-C₆)alkenyl; (C₁-C₆)alkoxy; OH; NO₂; C≡N; C(═O)OR⁷; C(═O)NR⁷₂; NR⁷₂; NR⁷C(═O)(C₁-C₆)alkyl; NR⁷C(═O)O(C₁-C₆)alkyl; NR⁷C(═O)NR⁷₂; NR⁷SO₂(C₁-C₆)alkyl; SO₂NR⁷₂; OC(═O)(C₁-C₆)alkyl; O(C₂-C₆)alkylene-NR⁷₂; (C₂-C₆)alkylene-OR⁷; and (C₁-C₃)perfluoroalkyl;
R³ is hydrogen, C(═O)OR⁷, or C(═O)NR⁷₂;
A² is CH or N;
R⁵ is H or CR⁸R⁹R¹⁰;
each occurrence of R⁷ and R¹⁰ is independently selected from the group consisting of hydrogen, (C₁-C₆)cycloalkyl and (C₁-C₆)alkyl;
each occurrence of R⁸ and R⁹ is independently selected from the group consisting of (C₁-C₆)cycloalkyl and (C₁-C₆)alkyl; or R⁸ and R⁹ are bound to the same carbon atom and linked as to form a divalent group selected from the group consisting of ethane-1,2-diyl, propane-1,3-diyl, butane-1,4-diyl, pentane-1,5-diyl, hexane-1,6-diyl and heptane-17-diyl; wherein said bivalent group is optionally substituted with at least one (C₁-C₆)alkyl group;
m is independently at each occurrence 1, 2, or 3;
n is 0, 1, or 2;
p is independently at each occurrence 2 or 3; and
q is independently at each occurrence 1 or 2;
a compound of formula III: 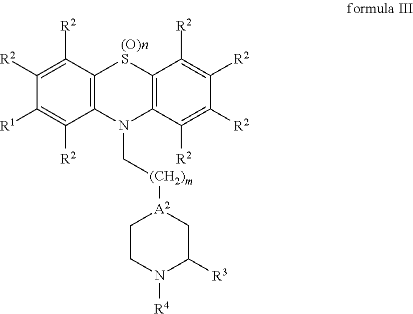
wherein:
each occurrence of R¹ and R² is independently selected from the group consisting of hydrogen, halogen, (C₁-C₆)alkyl; (C₁-C₆)alkenyl; (C₁-C₆)alkoxy; OH; NO₂; C≡N; C(═O)OR⁵; C(═O)NR⁵₂; NR⁵₂; NR⁵C(═O)(C₁-C₆)alkyl; NR⁵C(═O)O(C₁-C₆)alkyl; NR⁵C(═O)NR⁵₂; NR⁵SO₂(C₁-C₆)alkyl; SO₂NR⁵₂; OC(═O)(C₁-C₆)alkyl; O(C₂-C₆)alkylene-NR⁵₂; (C₂-C₆)alkylene-OR⁵; and (C₁-C₃)perfluoroalkyl;
R³ is hydrogen, C(═O)OR⁵, or C(═O)N(R⁵)₂;
A² is CH or N;
R⁴ is —(CR⁵₂)ₚN(R⁵)C(═O)—CR⁶R⁷R⁸; —(CR⁵₂)ₚO(CR⁵₂)ₚN(R⁵)C(═O)—CR⁶R⁷R⁸; —(CR⁵₂)ₚN(R⁵)(CR⁵₂)ₚN(R⁵)C(═O)—CR⁶R⁷R⁸; or —(CR⁵₂)ₚN(R⁵)C(═O)(CR⁵₂)ₚN(R⁵)C(═O)—CR⁶R⁷R⁸;
each occurrence of R⁵ and R⁶ is independently selected from the group consisting of hydrogen, (C₁-C₆)alkyl and (C₁-C₆)cycloalkyl;
R⁷ is (C₁-C₆)alkyl or (C₁-C₆)cycloalkyl; or R⁶ and R⁷ are bound to the same carbon atom and linked as to form a divalent group selected from the group consisting of ethane-1,2-diyl, propane-1,3-diyl, butane-1,4-diyl, pentane-1,5-diyl, hexane-1,6-diyl and heptane-17-diyl; wherein said bivalent group is optionally substituted with at least one (C₁-C₆)alkyl group;
R⁸ is (C₁-C₆)alkyl, —N(R⁵)C(═O)R⁵, or —N(R⁵)S(═O)₂R⁷;
m is independently at each occurrence 1, 2, or 3;
n is 0, 1, or 2; and,
p is independently at each occurrence 1, 2 or 3;
a salt thereof and mixtures thereof thereby treating said disease in said mammal.

24. A method of preventing or treating PAH in a mammal, said method comprising to said mammal a therapeutically effective amount of a pharmaceutically acceptable composition comprising a compound selected from the group comprising:
a compound of formula II: 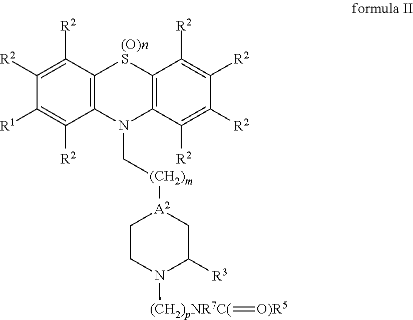
wherein:
each occurrence of R¹ and R² is independently selected from the group consisting of hydrogen, halogen, (C₁-C₆)alkyl; (C₁-C₆)alkenyl; (C₁-C₆)alkoxy; OH; NO₂; C≡N; C(═O)OR⁷; C(═O)NR⁷₂; NR⁷₂; NR⁷C(═O)(C₁-C₆)alkyl; NR⁷C(═O)O(C₁-C₆)alkyl; NR⁷C(═O)NR⁷₂; NR⁷SO₂(C₁-C₆)alkyl; SO₂NR⁷₂; OC(═O)(C₁-C₆)alkyl; O(C₂-C₆)alkylene-NR⁷₂; (C₂-C₆)alkylene-OR⁷; and (C₁-C₃)perfluoroalkyl;
R³ is hydrogen, C(═O)OR⁷, or C(═O)NR⁷₂;
A² is CH or N;
R⁵ is H or CR⁸R⁹R¹⁰;
each occurrence of R⁷ and R¹⁰ is independently selected from the group consisting of hydrogen, (C₁-C₆)cycloalkyl and (C₁-C₆)alkyl;
each occurrence of R⁸ and R⁹ is independently selected from the group consisting of (C₁-C₆)cycloalkyl and (C₁-C₆)alkyl; or R⁸ and R⁹ are bound to the same carbon atom and linked as to form a divalent group selected from the group consisting of ethane-1,2-diyl, propane-1,3-diyl, butane-1,4-diyl, pentane-1,5-diyl, hexane-1,6-diyl and heptane-17-diyl; wherein said bivalent group is optionally substituted with at least one (C₁-C₆)alkyl group;
m is independently at each occurrence 1, 2, or 3;
n is 0, 1, or 2;
p is independently at each occurrence 2 or 3; and
q is independently at each occurrence 1 or 2;
a compound of formula III: 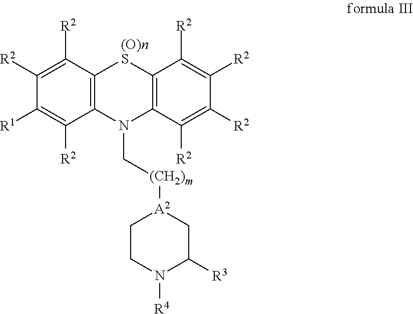
wherein:
each occurrence of R¹ and R² is independently selected from the group consisting of hydrogen, halogen, (C₁-C₆)alkyl; (C₁-C₆)alkenyl; (C₁-C₆)alkoxy; OH; NO₂; C≡N; C(═O)OR⁵; C(═O)NR⁵₂; NR⁵₂; NR⁵C(═O)(C₁-C₆)alkyl; NR⁵C(═O)O(C₁-C₆)alkyl; NR⁵C(═O)NR⁵₂; NR⁵SO₂(C₁-C₆)alkyl; SO₂NR⁵₂; OC(═O)(C₁-C₆)alkyl; O(C₂-C₆)alkylene-NR⁵₂; (C₂-C₆)alkylene-OR⁵; and (C₁-C₃)perfluoroalkyl;
R³ is hydrogen, C(═O)OR⁵, or C(═O)N(R⁵)₂;
A² is CH or N;
R⁴ is —(CR⁵₂)ₚN(R⁵)C(═O)—CR⁶R⁷R⁸; —(CR⁵₂)ₚO(CR⁵₂)ₚN(R⁵)C(═O)—CR⁶R⁷R⁸; —(CR⁵₂)ₚN(R⁵)(CR⁵₂)ₚN(R⁵)C(═O)—CR⁶R⁷R⁸; or —(CR⁵₂)ₚN(R⁵)C(═O)(CR⁵₂)ₚN(R⁵)C(═O)—CR⁶R⁷R⁸;
each occurrence of R⁵ and R⁶ is independently selected from the group consisting of hydrogen, (C₁-C₆)alkyl and (C₁-C₆)cycloalkyl;
R⁷ is (C₁-C₆)alkyl or (C₁-C₆)cycloalkyl; or R⁶ and R⁷ are bound to the same carbon atom and linked as to form a divalent group selected from the group consisting of ethane-1,2-diyl, propane-1,3-diyl, butane-1,4-diyl, pentane-1,5-diyl, hexane-1,6-diyl and heptane-17-diyl; wherein said bivalent group is optionally substituted with at least one (C₁-C₆)alkyl group;
R⁸ is (C₁-C₆)alkyl, —N(R⁵)C(═O)R⁵, or —N(R⁵)S(═O)₂R⁷;
m is independently at each occurrence 1, 2, or 3;
n is 0, 1, or 2; and,
p is independently at each occurrence 1, 2 or 3;
a salt thereof and mixtures thereof, thereby preventing or treating PAH in said mammal.
